(19) United States
(12) Patent Application Publication
(10) Pub. No.: US 2026/0199365 A1
(43) Pub. Date: Jul. 16, 2026

(54) BIS-SULFONAMIDE SHIP1 ACTIVATORS

(71) Applicants: The Research Foundation for the State University of New York, Albany, NY (US); SYRACUSE UNIVERSITY, Syracuse, NY (US)

(72) Inventors: William Kerr, Syracuse, NY (US); John Chisholm, Syracuse, NY (US)

(21) Appl. No.: 19/130,033

(22) PCT Filed: Nov. 14, 2023
(86) PCT No.: PCT/US2023/037264

§ 371 Date: May 14, 2025

Related U.S. Application Data
(60) Provisional application No. 63/425,601, filed on Nov. 15, 2022.

(51) Int. Cl. A61K 31/541; A61K 31/381; A61K 31/454

(52) U.S. Cl. CPC A61K 31/541; A61K 31/381; A61K 31/454

(57) ABSTRACT

Provided are compounds and pharmaceutically acceptable salt, solvate and/or derivative thereof. Further, provided are methods of treating a disease, disorder or condition mediated or treatable by activation of SHIP1 comprising administering a SHIP1 activator compound or a pharmaceutically acceptable salt, solvate or derivative thereof. The compound or a pharmaceutically acceptable salt, solvate or derivative thereof may be used in the treatment of SHIP1 mediated disease, disorder or conditions including inflammatory bowel disease (IBD), Crohn′ disease, COPD, ulcerative colitis, arthritis, and Alzheimer's Disease.

### CROSS-REFERENCE TO RELATED APPLICATIONS

The present disclosure claims priority or the benefit under 35 U.S.C. § 119 of U.S. provisional application No. 63/425,601 filed Nov. 15, 2022, herein entirely incorporated by reference.

### STATEMENT OF GOVERNMENTAL SUPPORT

This invention was made with government support under grant number AG059717, awarded by the National Institute of Health. The government has certain rights in the invention.

### FIELD OF THE INVENTION

The present disclosure provides compositions that modulate the abnormal signaling in the PI3K pathway, as well as methods using such compositions for use in treating or ameliorating the effects of a medical condition in a subject.

### BACKGROUND

The phosphatidylinositol 3,4,5-trisphosphate 5-phosphatase (SHIP) enzyme opposes the activity of PI3K, and therefore is of interest in the treatment of inflammatory disorders like Crohn's disease, COPD and arthritis. SH-2 containing inositol 5′ polyphosphatase 1 (SHIP1) promotes phagolysosomal degradation of lipids by microglia has also suggested that the enzyme may be a target in Alzheimer's disease. Therefore, small molecules that increase SHIP1 activity may have benefits in these areas. We have discovered bis-sulfonamide compounds that increase the enzymatic activity of SHIP1. A series of similar SHIP1 activators have been synthesized and evaluated. Both the thiophene and the thiomorpholine in the parent structure can be replaced by groups without a low valent sulfur, which provides a way to access activators that are less prone to oxidative degradation.

### SUMMARY OF THE INVENTION

The present disclosure now provides SHIP activators including the appropriate pharmacological properties that are safe and therapeutically useful while providing effective inhibition. Such compounds are excellent for treating the disease states where inhibition could play a role. Moreover, the present disclosure provides one or more specific bis-sulfonamide compounds that increase the enzymatic activity of SHIP1 and/or SHIP2. Further, the present disclosure now provides for SHIP activation using pharmaceutically acceptable SHIP activator compounds to robustly reduce and/or ameliorate acute or chronic inflammatory diseases.

In embodiments, the present disclosure provides a method for treating a chronic or acute inflammatory disease comprising administering to a subject in need thereof, a therapeutically effective amount of one or more compounds that activate phosphatidylinositol 3,4,5-trisphosphate 5-phosphatase (SHIP), or an isoform thereof.

In embodiments, the present disclosure includes a method of treating inflammatory disease, such as, chronic obstructive pulmonary disease (COPD), inflammatory bowel disease (IBD), rheumatoid arthritis (RA), or septic shock.

In embodiments, the present disclosure includes a method of treating inflammatory disease, such as, Alzheimer disease.

In embodiments, the present disclosure includes a method of treating inflammatory disease, wherein treating comprises administering a therapeutically effective amount of one or more SHIP activator compounds to the subject.

In embodiments, the present disclosure includes a method of treating inflammatory disease, wherein the SHIP activator compound is

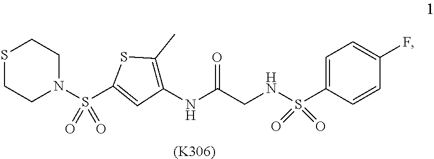 [SMILES: Cc1sc(S(=O)(=O)N2CCSCC2)cc1NC(=O)CNS(=O)(=O)c1ccc(F)cc1 ; "(K306)"]

or an analog thereof.

In embodiments, the present disclosure includes a method of treating inflammatory disease, wherein the one or more SHIP activator compound is selected from the group consisting of:

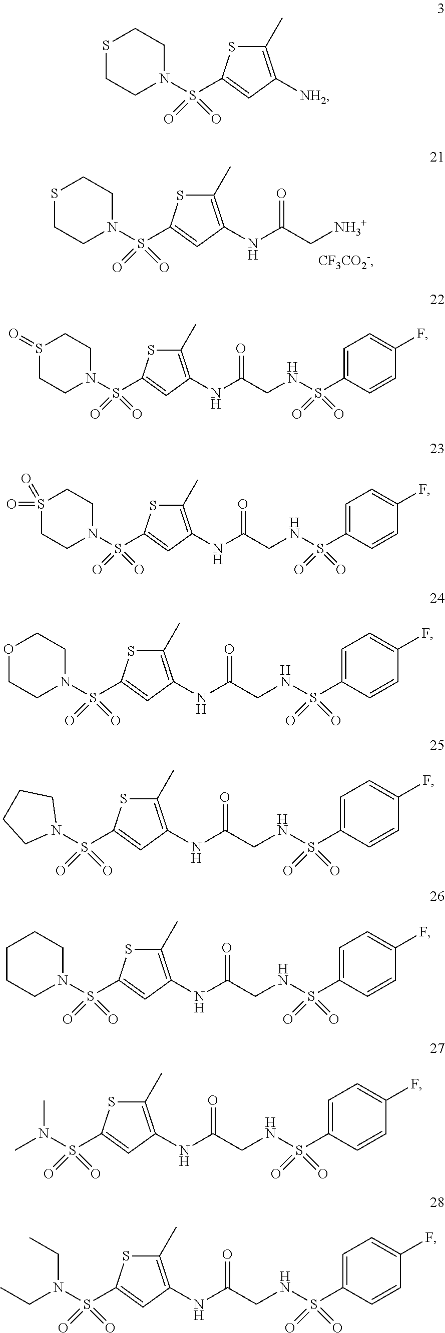

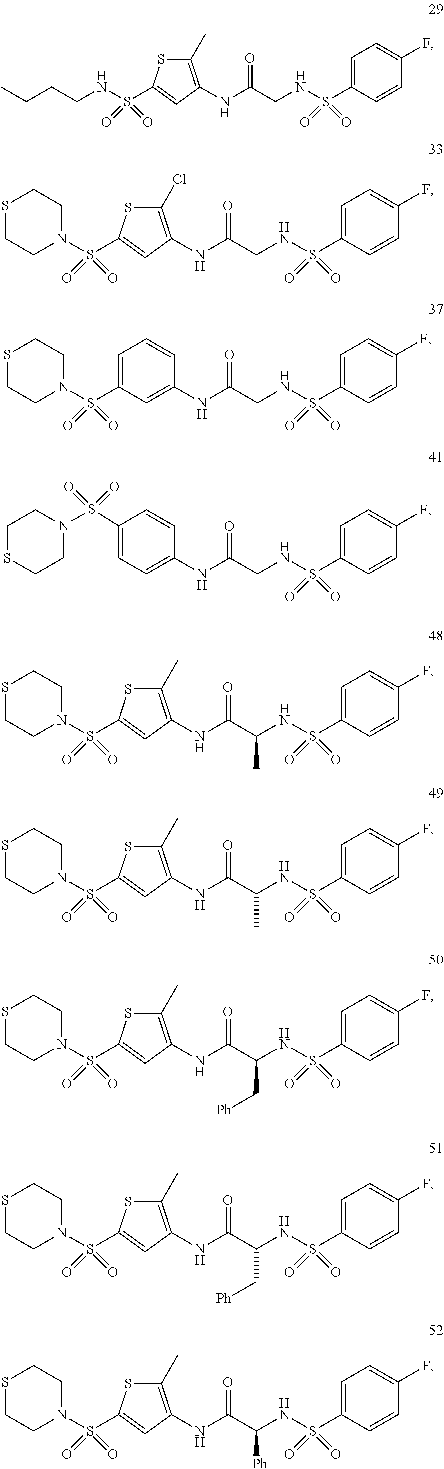

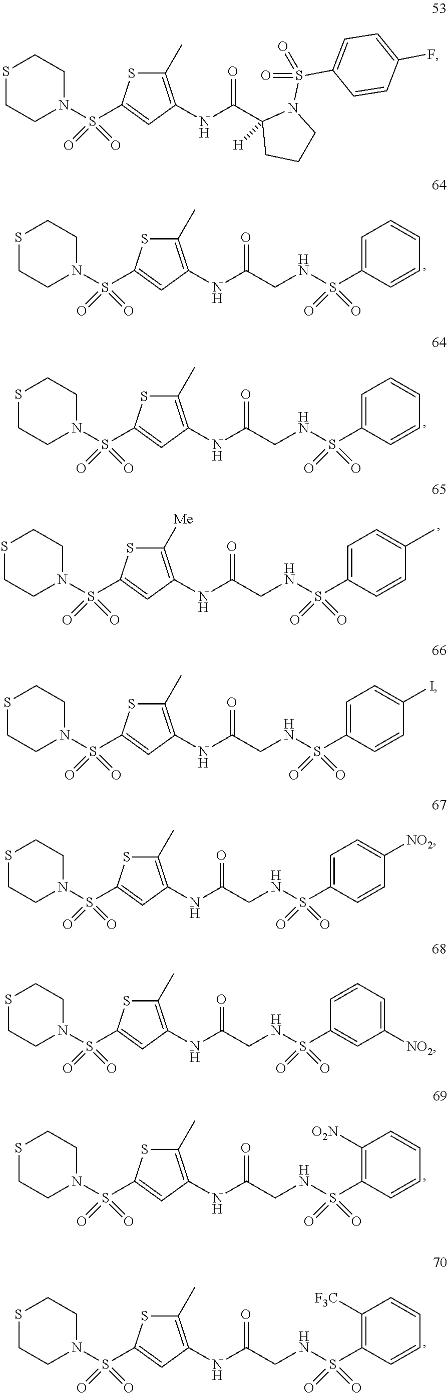

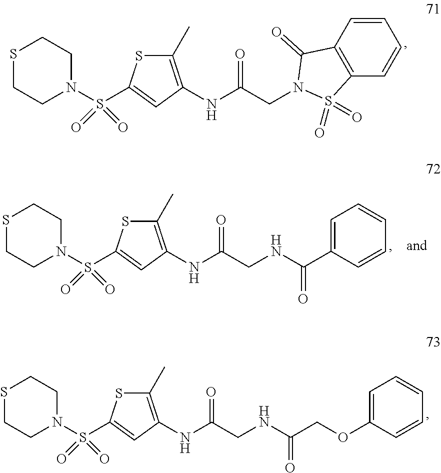 [SMILES: Cc1sc(S(=O)(=O)N2CCSCC2)cc1NC(=O)CN1C(=O)c2ccccc2S1(=O)=O ; Cc1sc(S(=O)(=O)N2CCSCC2)cc1NC(=O)CNC(=O)c1ccccc1 ; Cc1sc(S(=O)(=O)N2CCSCC2)cc1NC(=O)CNC(=O)COc1ccccc1]

or a pharmaceutically acceptable salt thereof, and combinations thereof.

In embodiments, the present disclosure includes pharmaceutical anti-inflammatory compositions comprising one or more SHIP activators selected from the group consisting of:

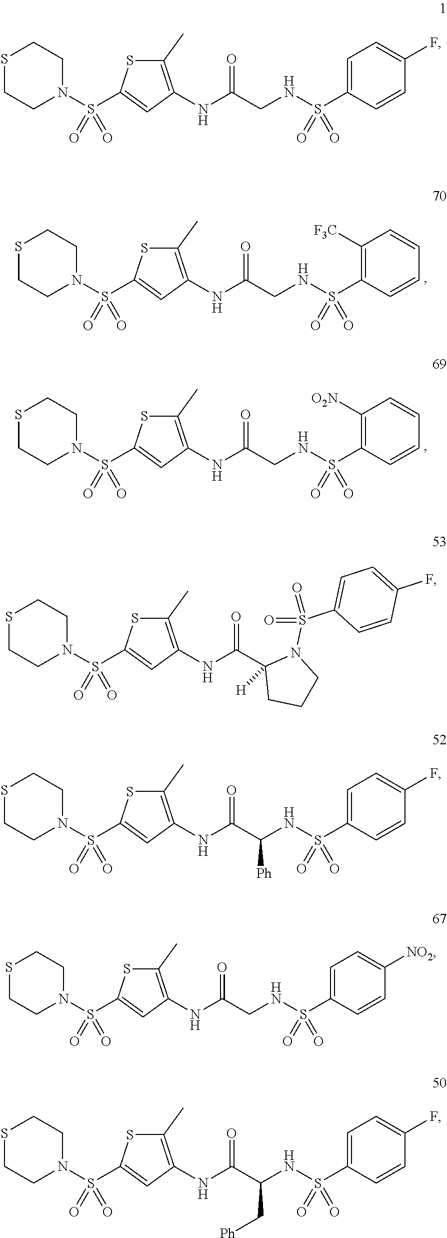

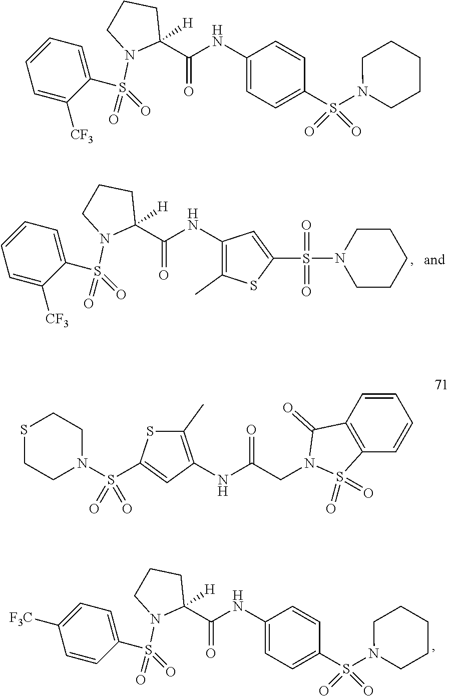 [SMILES: O=C(Nc1ccc(S(=O)(=O)N2CCCCC2)cc1)[C@@H]1CCCN1S(=O)(=O)c1ccccc1C(F)(F)F ; Cc1sc(S(=O)(=O)N2CCCCC2)cc1NC(=O)[C@@H]1CCCN1S(=O)(=O)c1ccccc1C(F)(F)F ; Cc1sc(S(=O)(=O)N2CCSCC2)cc1NC(=O)CN1C(=O)c2ccccc2S1(=O)=O ; O=C(Nc1ccc(S(=O)(=O)N2CCCCC2)cc1)[C@@H]1CCCN1S(=O)(=O)c1ccc(C(F)(F)F)cc1]

or a pharmaceutically acceptable salt thereof, and combinations thereof.

In embodiments, the present disclosure includes a method of treating inflammatory disease, wherein SHIP activator compound activates phosphatidylinositol 3,4,5-trisphosphate 5-phosphatase isoform SHIP1 and/or SHIP2.

In embodiments, the present disclosure includes a method of treating inflammatory disease, wherein SHIP activator compound activates SHIP1.

In embodiments, the present disclosure includes a method of treating inflammatory disease, wherein treating the subject with the one or more SHIP activator compounds is sequential.

In embodiments, the present disclosure includes a method of treating inflammatory disease, wherein treating the subject with the one or more SHIP activator compounds is simultaneous.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The illustrative aspects of the present disclosure are designed to solve the problems herein described and/or other problems not discussed.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 presents a diagrammatic scheme of the PI3K/PTEN Pathway and SHIP1.

FIG. 2 presents a schematic analysis of potential CYP oxidation sites on the K306 compound.

FIG. 3 presents an ELISA quantitation of IL-6 production in BV-2 cells after stimulation with LPS. BV-2 cells were plated and treated for 1 h with vehicle (Veh, 0.25% DMSO) or SHIP1 activator (10 μM in Veh) prior to addition of LPS (100 ng/ml). Unstimulated BV2 cells treated with Veh are shown as an additional control to assess LPS induction of IL-6. (A) Evaluation of 1, 26, 29 and 53. (B) In a separate experiment, 70, and 71 were evaluated under similar conditions. Bars indicate Mean G SEM; statistical tests: two-way ANOVA with Tukey correction, **** p<0.0001, ** p<0.01.

### DETAILED DESCRIPTION OF CERTAIN EMBODIMENTS

The disclosed method and compositions may be understood more readily by reference the following detailed description of particular embodiments and the Example included therein and to the Figures and their previous and following description.

Hereinafter, preferred embodiments of the present invention will be described in detail with reference to the accompanying drawings so that those of ordinary skill in the art can easily carry out the present invention. However, the present invention may be implemented in several different forms and is not limited or restricted by the following examples.

In order to clearly explain the present invention, detailed descriptions of portions that are irrelevant to the description or related known technologies that may unnecessarily obscure the gist of the present invention have been omitted, and in the present specification, reference symbols are added to components in each drawing. In this case, the same or similar reference numerals are assigned to the same or similar elements throughout the specification.

Also, terms or words used in this specification and claims should not be restrictively interpreted as ordinary meanings or dictionary-based meanings, but rather should be interpreted consistently with the concepts disclosed in the present application, based on the principle that an inventor can act as his/her own lexicographer, defining terms so as to best describe and explain his or her invention.

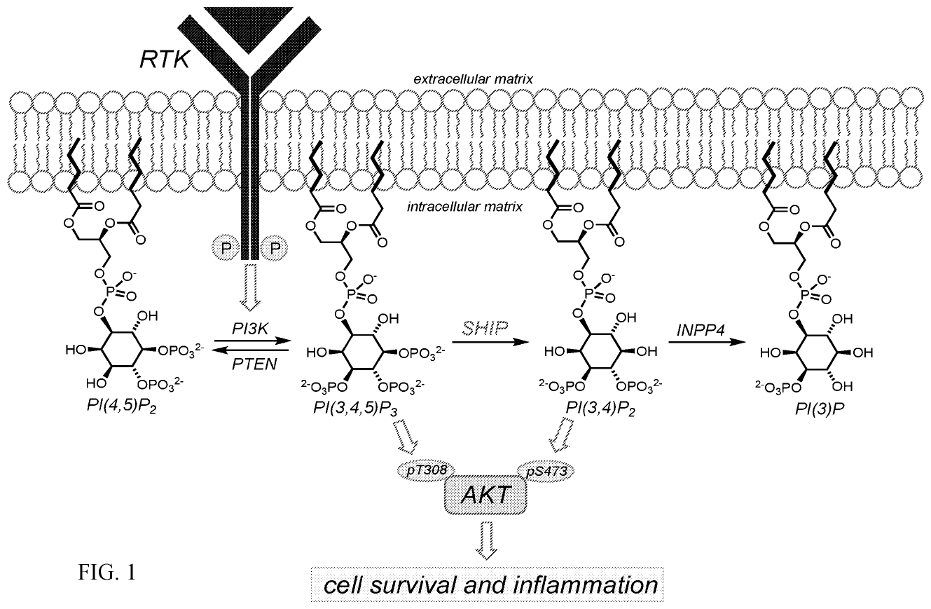

Inositol phospholipids are critical participants in the PI3K/AKT pathway, a key cell signaling pathway in eukaryotic cells. The phosphorylation pattern on the inositol acts as a recognition element for intracellular protein kinases, which, after binding, initiate phosphorylation cascades that contribute to signaling between the cell membrane and the nucleus. Inositol phosphorylation is tightly controlled by inositol kinases and phosphatases, as this signaling influences cell survival and inflammation. Enzymes like PI3K, PTEN, SHIP1/2 and INPP4A/B are known to metabolize inositol phospholipids (FIG. 1). The activity of phosphatidylinositol-3-kinases (PI3K) has been shown to have profound effects on cellular physiology. Once activated, PI3K rapidly synthesizes PI(3,4,5)P3 from phosphatidylinositol-4,5-bisphosphate (PI(4,5)P2) (see FIG. 1). PI(3,4,5)P3 then recruits and activates AKT, which phosphorylates other protein kinases, transmitting signals from the cell membrane to the nucleus. The PH domain of AKT recognizes both PI(3,4,5)P3 and PI(3,4)P2, however, and therefore the activity of this key protein kinase may be influenced by modulation of either PI3K or SHIP (SH2 containing inositol 5′-phosphatase). The PH domain of AKT has a higher affinity for PI(3,4)P2 than PI(3,4,5)P3, and both phosphoinositol species participate in the activation of AKT and its downstream signaling. In contrast to PTEN (phosphatase and tensin homolog), which directly reverses the PI3K reaction to generate PI(4,5)P2 from PI(3,4,5)P3, SHIP generates PI(3,4)P2, which can be further dephosphorylated by INPP4.

Abnormal signaling in the PI3K pathway has been linked to many diseases and disorders. Modulation of the kinases and phosphatases that control the levels of these phospholipids with small molecules may provide a means to address a number of these areas. For example, SHIP1 activators may have applications as anti-inflammatories. The progression of IBD and Crohn's disease have been linked to a deficiency of SHIP1 in the intestine. A role for SHIP1 in IBD was first implicated in SHIP1 knockout mice, which develop ileitis in the small intestine accompanied with neutrophilia and greatly reduced T cell numbers. Genetic studies in humans have also observed that SHIP1-deficiency correlates strongly with IBD. One recent study observed that approximately 15% of adult IBD patients were SHIP1-deficient in their digestive tracts. Other independent analyses have reported a decrease in SHIP1 expression across a spectrum of Crohn's patients. These findings of partial SHIP1 deficiency offers an opportunity for intervention in Crohn's and IBD patients utilizing SHIP1 activators.

Changes in SHIP1 activity have also been linked to Alzheimer's disease. A number of genome wide association studies (GWAS) have identified single nucleotide polymorphisms (SNP) s in the INPP5D (SHIP1) gene linked to Alzheimer disease (AD) risk. Both isoforms of the phosphatidylinositol 3,4,5-trisphosphate 5-phosphatase (SHIP) enzyme, i.e., SHIP1 and SH2-containing 5′-inositol phosphatase 2 (SHIP2) are expressed at the protein level in microglia, and thus both SHIP paralogs could potentially limit microglial homeostatic functions that control amyloidosis and remove dead or dying neurons. SHIP1 expression is increased in the brains of late-onset AD patients, and also in 5×FAD mice which are a common AD model system. While this would seem to implicate SHIP inhibition as a potential treatment option for AD, recent studies implicate a more complex situation. Two groups have published analyses of SHIP1 mutations in 5×FAD mice but arrived at differing conclusions. One study, which used germline SHIP1 haploinsufficient 5×FAD mice, found that plaque burden was reduced, and that behavioral studies in these haploinsufficient mice demonstrated a preserved cognitive function, supporting the use of SHIP inhibitors. The other study used a conditional knockout of SHIP1 restricted to microglia and macrophages, finding that SHIP1 is required to limit Aβ plaque burden, which would implicate the use of SHIP1 activators as a treatment option. The role of SHIP1 in AD is therefore still under investigation, with potential benefits of up- or downregulation of the enzyme still being evaluated. One hypothesis is that up- or downregulation of SHIP1 enzymatic activity could be beneficial depending on the disease stage. For example, in early stages patients may benefit from enhanced microglial homeostatic function that is induced by activation, while in late stages antagonism of SHIP1 may instead be beneficial to reduce damage from dysfunctional microglia.

New small molecule SHIP1 activators are needed to explore the effects of these molecules in IBD and AD model systems to define the role of SHIP in these disorders. While several SHIP1 activators (often referred to as enzyme agonists; as used herein, the terms SHIP activators and SHIP agonists are interchangeable) are known, and have demonstrated activity as anti-inflammatories, most suffer from poor water solubility or low efficacy. Recently we discovered a bis-sulfonamide, i.e., 2-(4-Fluorobenzenesulfonamido)-N-[2-methyl-5-(thiomorpholine-4-sulfonyl)thiophen-3-yl]acetamide (K306, 1) which had significant activity as a SHIP1 activator. The K306 structure is related to a series of sulfonamide based endothelial lipase inhibitors. As the molecule came from screening, no information was available about structure activity relationships in this SHIP1 activator. Sulfonamides have been used previously as phosphate mimics in the development of phosphatase inhibitors however, so it is possible that one of the sulfonamides in the K306 structure is mimicking the one of the phosphates on PI(3,4)P2, the endogenous ligand for allosteric activation of SHIP1. Interestingly, K306 was shown to bind to a new allosteric site on the protein, as other activators lost activity when the C2 domain of the protein was deleted, yet K306 maintained agonism with this C2-less SHIP1 protein. K306 was shown to enhance phagolysosomal degradation of synaptosomes and dead neurons by microglia in a SHIP1-dependent manner, revealing a new function for SHIP1 in the brain that could be exploited to enhance microglial function in inflammatory disease states. Given that K306 provides a new class of SHIP1 activators with a unique binding site that could have implications in Crohn's disease and Alzheimer's disease, synthetic studies were initiated on the structure.

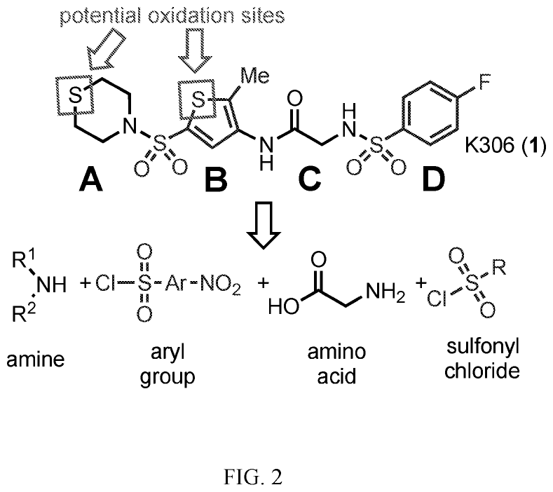

Initial testing of K306 demonstrated significant SHIP1 activation, with increases of 33% and 6% when tested at 500 μM and 250 μM concentrations in the malachite green assay for phosphatase activity. Further studies determined that K306 suffered a short-half life in vivo, requiring a multiple dosing regimen in order to achieve anti-inflammatory responses in mice. This is believed to be due to rapid oxidation of the sulfur functional groups in K306 by cytochrome P450 enzymes which are prevalent in mice. Evaluation of the K306 structure by SmartCYP, a method employing density functional theory to identify the most likely sites of CYP metabolism, showed that there was a high likelihood of oxidation, particularly of the sulfide present in the thiomorpholine subunit (see FIG. 2). A lower possibility of oxidation on the thiophene was noted, as the aromaticity of the system and steric effects should slow these reactions. The thiophene is still a concern, however, as this heterocycle is known to undergo oxidation by CYP enzymes and can lead to toxic metabolites.

Synthetic studies were initiated on the K306 structure to investigate structure activity relationships and determine if the oxidation of the thiomorpholine leads to inactive derivatives. In considering the K306 structure, the molecule may be readily divided into four distinct quadrants: an alkyl sulfonamide quadrant A, the aromatic linker quadrant B, the amino acid quadrant C, and an aryl sulfonamide quadrant D. Once the most active constituent at each quadrant was determined, hybrid molecules that could have even greater potency may be explored by combining the best subunits.

Initially the amine of sector A was changed to determine the necessity of the thiomorpholine and if the oxidation of this group was leading to inactivation. The synthesis of these analogs followed the reported synthesis of K306, beginning with a sulfonamide synthesis using sulfonyl chloride 2 and some amines (Scheme 1). Reduction of the nitro group on these intermediates with iron powder in acetic acid then gave the corresponding aromatic amines. Amide formation with Boc-glycine mediated by HATU provided the carbamates. Removal of the Boc protecting groups with TFA (which produces amine salt 21) followed by sulfonamide formation with 4-fluorobenzenesulfonyl chloride gave the desired bis-sulfonamides.

TABLE 1
Synthesis of K306 analogs varying section A.

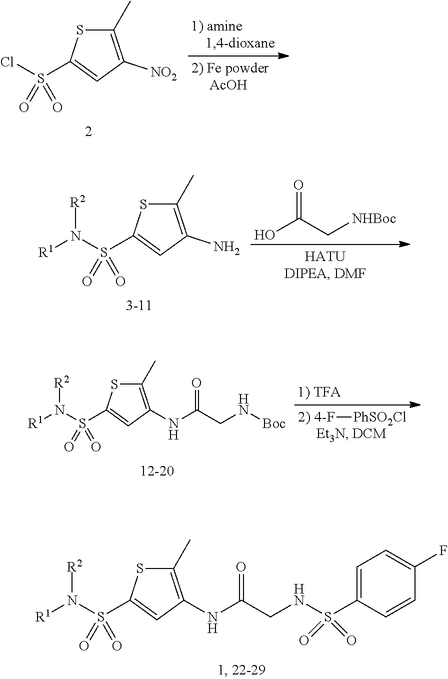

| Entry | Aminothiophene | Amide |
|---|---|---|
| 1 | 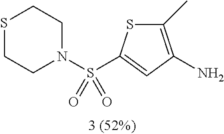 [SMILES: Cc1sc(S(=O)(=O)N2CCSCC2)cc1N] | 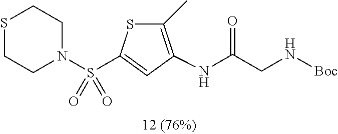 [SMILES: Cc1sc(S(=O)(=O)N2CCSCC2)cc1NC(=O)CNC(=O)OC(C)(C)C] |
| 2 | | |
| 3 | 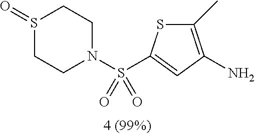 [SMILES: Cc1sc(S(=O)(=O)N2CCS(=O)CC2)cc1N] | 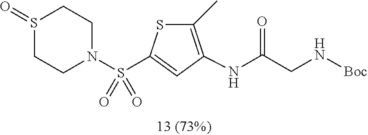 [SMILES: Cc1sc(S(=O)(=O)N2CCS(=O)CC2)cc1NC(=O)CNC(=O)OC(C)(C)C] |
| 4 | 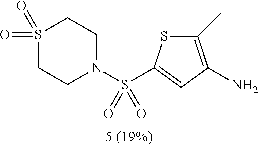 [SMILES: Cc1sc(S(=O)(=O)N2CCS(=O)(=O)CC2)cc1N] | 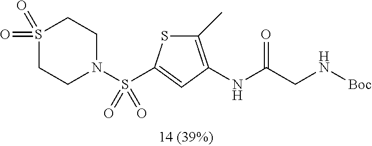 [SMILES: Cc1sc(S(=O)(=O)N2CCS(=O)(=O)CC2)cc1NC(=O)CNC(=O)OC(C)(C)C] |
| 5 | 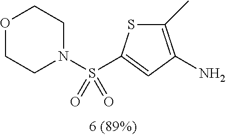 [SMILES: Cc1sc(S(=O)(=O)N2CCOCC2)cc1N] | 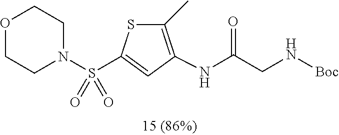 [SMILES: Cc1sc(S(=O)(=O)N2CCOCC2)cc1NC(=O)CNC(=O)OC(C)(C)C] |
| 6 | 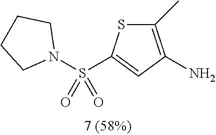 | 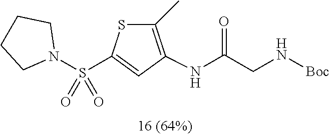 [SMILES: Cc1sc(S(=O)(=O)N2CCCC2)cc1NC(=O)CNC(=O)OC(C)(C)C] |
| 7 | 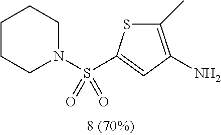 [SMILES: Cc1sc(S(=O)(=O)N2CCCCC2)cc1N] | 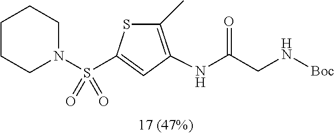 [SMILES: Cc1sc(S(=O)(=O)N2CCCCC2)cc1NC(=O)CNC(=O)OC(C)(C)C] |
| 8 | 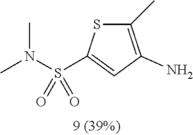 | 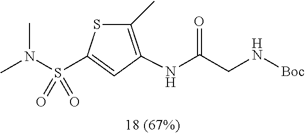 [SMILES: Cc1sc(S(=O)(=O)N(C)C)cc1NC(=O)CNC(=O)OC(C)(C)C] |
| 9 | 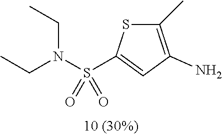 | 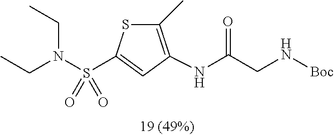 [SMILES: CCN(CC)S(=O)(=O)c1cc(NC(=O)CNC(=O)OC(C)(C)C)c(C)s1] |
| 10 | 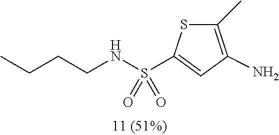 | 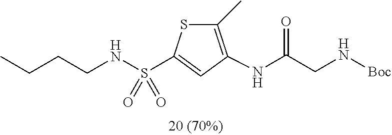 [SMILES: CCCCNS(=O)(=O)c1cc(NC(=O)CNC(=O)OC(C)(C)C)c(C)s1] |

| Entry | Bis-Sulfonamide |
|---|---|
| 1 | 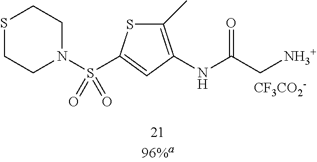 |
| 2 | 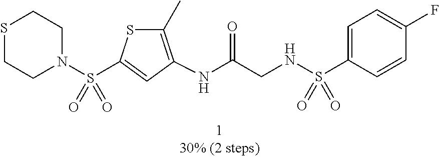 [SMILES: Cc1sc(S(=O)(=O)N2CCSCC2)cc1NC(=O)CNS(=O)(=O)c1ccc(F)cc1] |
| 3 | 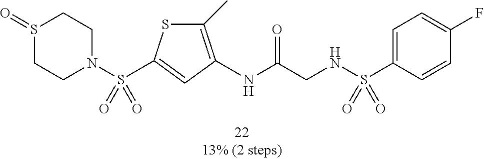 [SMILES: Cc1sc(S(=O)(=O)N2CCS(=O)CC2)cc1NC(=O)CNS(=O)(=O)c1ccc(F)cc1] |
| 4 | 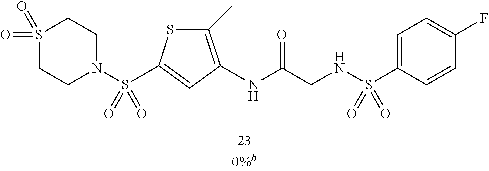 [SMILES: Cc1sc(S(=O)(=O)N2CCS(=O)(=O)CC2)cc1NC(=O)CNS(=O)(=O)c1ccc(F)cc1] |
| 5 | 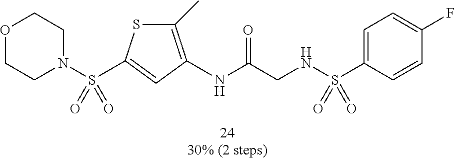 [SMILES: Cc1sc(S(=O)(=O)N2CCOCC2)cc1NC(=O)CNS(=O)(=O)c1ccc(F)cc1] |
| 6 | 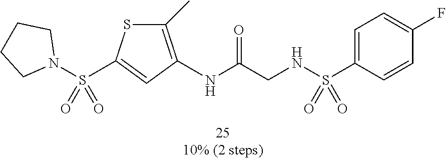 [SMILES: Cc1sc(S(=O)(=O)N2CCCC2)cc1NC(=O)CNS(=O)(=O)c1ccc(F)cc1] |
| 7 | 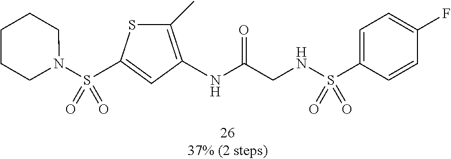 [SMILES: Cc1sc(S(=O)(=O)N2CCCCC2)cc1NC(=O)CNS(=O)(=O)c1ccc(F)cc1] |
| 8 | 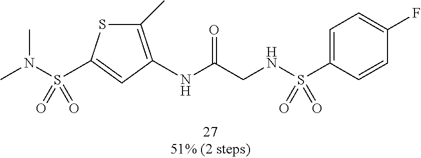 [SMILES: Cc1sc(S(=O)(=O)N(C)C)cc1NC(=O)CNS(=O)(=O)c1ccc(F)cc1] |
| 9 | 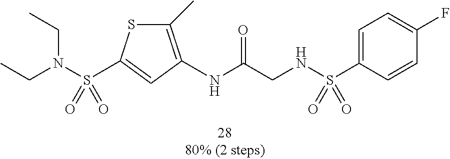 [SMILES: CCN(CC)S(=O)(=O)c1cc(NC(=O)CNS(=O)(=O)c2ccc(F)cc2)c(C)s1] |
| 10 | 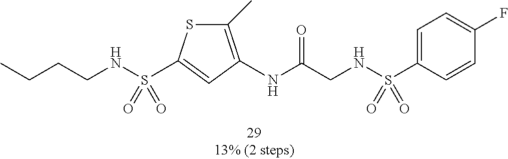 [SMILES: CCCCNS(=O)(=O)c1cc(NC(=O)CNS(=O)(=O)c2ccc(F)cc2)c(C)s1] |

^{a}Amine 21 is the product of the TFA deprotection and was isolated for testing before sulfonamide formation.
^{b}The sulfone 23 was insoluble (see below).

While most of the analogs in Table 1 were easy to handle, attempts to isolate the thiomorpholine sulfone 23 were problematic. During the final sulfonamide formation to access 23 an insoluble solid was formed and filtered from the reaction mixture. This solid was postulated to be the sulfone 23, but was insoluble in any solvent (DMSO, acetone, methanol, chloroform, and water) so verification of the structure by NMR was impossible. A combustion analysis did match the formula for the desired product, however given the insolubility of this substance it was not evaluated further.

The structure of the aryl group in section B of K306 was also varied. The synthesis of these systems began with the requisite nitro substituted sulfonyl chloride (30, 34 and 38, Scheme 1), which was then used in a sulfonamide formation with thiomorpholine. Attempts to directly couple a glycine N-sulfonamide to the aromatic amine were unsuccessful, so completion of the western section of the molecule was accomplished in a stepwise fashion. Reduction of the respective nitro groups provided the amines 31, 35 and 39. An amide coupling was performed with Boc-glycine as the coupling partner. Deprotection of the Boc protecting group and sulfonamide formation with 4-fluorobenzenesulfonyl chloride then gave analogs 33, 37 and 41.

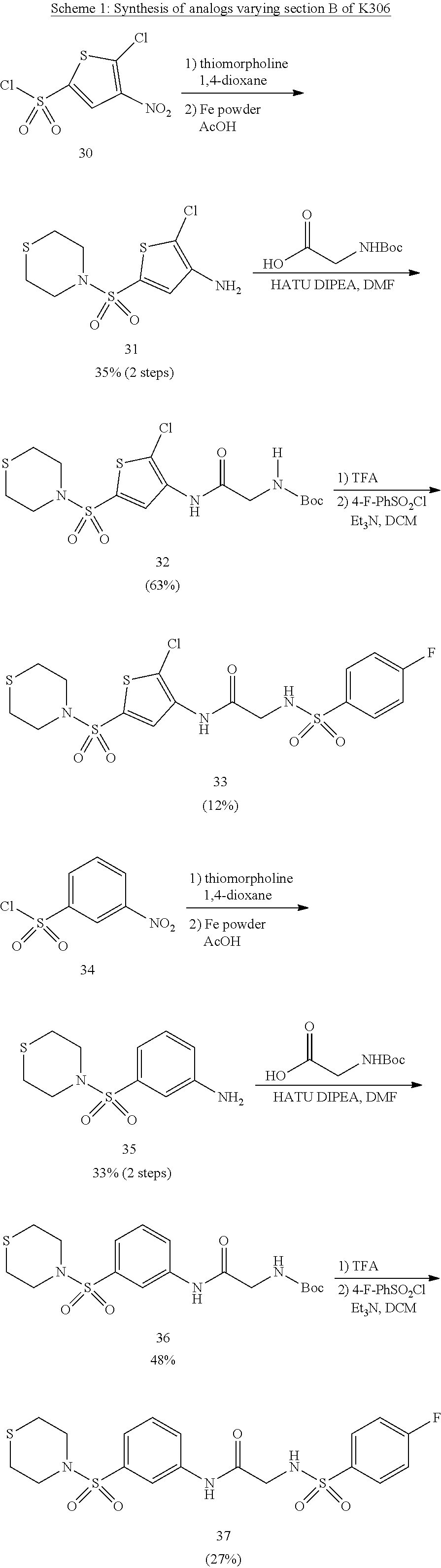

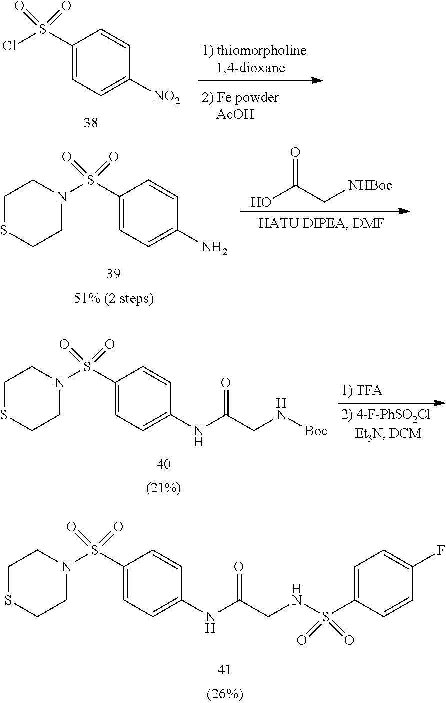 [SMILES: O=[N+]([O-])c1ccc(S(=O)(=O)Cl)cc1 ; Nc1ccc(S(=O)(=O)N2CCSCC2)cc1 ; CC(C)(C)OC(=O)NCC(=O)O ; CC(C)(C)OC(=O)NCC(=O)Nc1ccc(S(=O)(=O)N2CCSCC2)cc1 ; O=C(CNS(=O)(=O)c1ccc(F)cc1)Nc1ccc(S(=O)(=O)N2CCSCC2)cc1]

The amino acid sector of the activator (section C) was also modified. This was accomplished by using different amino acids as coupling partners following the general pathway shown in Table 2 below. The amine 3 was coupled with a Boc-protected amino acid utilizing HATU as the coupling agent. Removal of the Boc protecting group was then followed by sulfonamide formation with 4-fluorobenzenesulfonyl chloride. This led to the synthesis of compounds 48-53.

TABLE 2
Synthesis of K306 analogs varying section C:

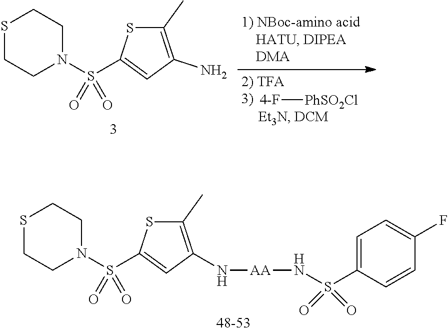

| Entry | Amino Acid | Product | Yield^{a} |
|---|---|---|---|
| 1 | 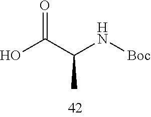 | 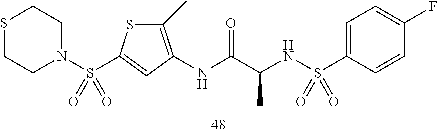 [SMILES: Cc1sc(S(=O)(=O)N2CCSCC2)cc1NC(=O)[C@H](C)NS(=O)(=O)c1ccc(F)cc1] | 21% |
| 2 | 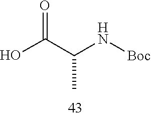 | 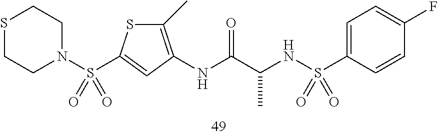 [SMILES: Cc1sc(S(=O)(=O)N2CCSCC2)cc1NC(=O)[C@@H](C)NS(=O)(=O)c1ccc(F)cc1] | 21% |
| 3 | 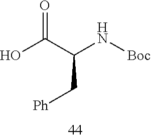 [SMILES: CC(C)(C)OC(=O)N[C@@H](Cc1ccccc1)C(=O)O] | 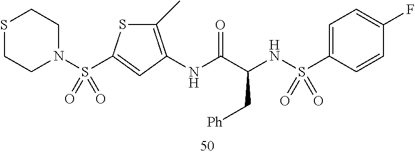 [SMILES: Cc1sc(S(=O)(=O)N2CCSCC2)cc1NC(=O)[C@H](Cc1ccccc1)NS(=O)(=O)c1ccc(F)cc1] | 15% |
| 4 | 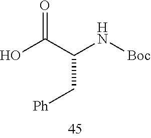 | 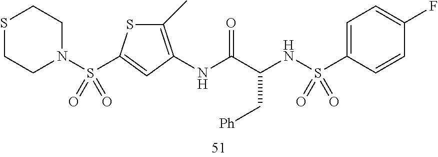 [SMILES: Cc1sc(S(=O)(=O)N2CCSCC2)cc1NC(=O)[C@@H](Cc1ccccc1)NS(=O)(=O)c1ccc(F)cc1] | 18% |
| 5 | 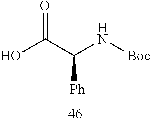 [SMILES: CC(C)(C)OC(=O)N[C@H](C(=O)O)c1ccccc1] | 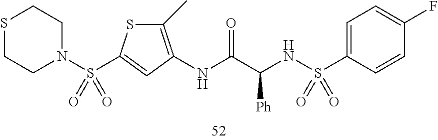 [SMILES: Cc1sc(S(=O)(=O)N2CCSCC2)cc1NC(=O)[C@@H](NS(=O)(=O)c1ccc(F)cc1)c1ccccc1] | 43% |
| 6 | 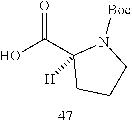 [SMILES: CC(C)(C)OC(=O)N1CCC[C@H]1C(=O)O] | 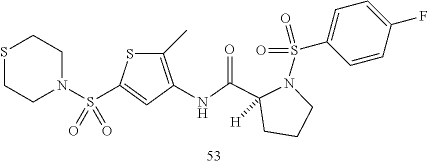 [SMILES: Cc1sc(S(=O)(=O)N2CCSCC2)cc1NC(=O)[C@@H]1CCCN1S(=O)(=O)c1ccc(F)cc1] | 54% |

^{a}Isolated yield over 3 steps.

Additionally, analogs of K306 with different groups in sector D were also synthesized starting from amine 21 (Table 3). Modification of this region of the molecule was accomplished by adding different electrophiles to the amine 21. These additions were not restricted to sulfonyl chlorides as coupling partners, with acid chlorides, chloroformates, isocyanates and isothiocyanates also being briefly investigated, leading to the synthesis of 64-73.

Evaluation of the effects of these analogs on SHIP1 utilizing the malachite green assay for phosphatase activity showed some interesting trends in reactivity (Table 4). Analogs without the sulfonamide end group in section C (3 and 21) had a reduced effect on SHIP activation. Sulfonamide 22, where the thiomorpholine sulfide has been oxidized to a sulfoxide, also showed no activity as a SHIP activator, which is consistent with oxidation of the sulfur being responsible for a short-half life in vivo. Interestingly, sulfoxide 22 showed some inhibition of SHIP1 instead of activation. As the endogenous ligand the allosteric activation of SHIP1 is the product PI(3,4)P2, and this is similar in structure to PI(3,4,5)P3, the active site and the allosteric binding site may share recognition elements that allow small structural changes to switch the role of the molecule from activator to inhibitor. Replacement of the thiomorpholine with a morpholine or a pyrrolidine (24 and 25) resulted in a loss of activity. The use of a more lipophilic piperidine did provide an active analog, however, as 26 was nearly as active as K306 (1). Opening the piperidine ring was then explored, with analogs 27-29. A rough trend that favored the less polar amines was noted with these analogs, which may indicate that this section of the inhibitor is binding in a nonpolar pocket of the protein.

TABLE 3
Synthesis of K306 analogs varying section D.

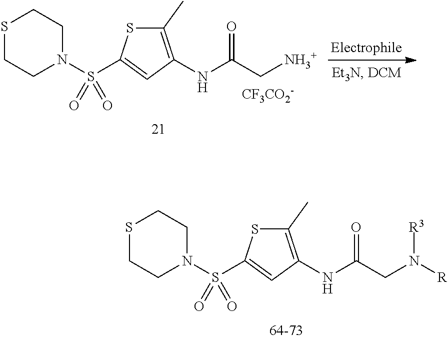

| Entry | Electrophile | Product | Yield |
|---|---|---|---|
| 1 | 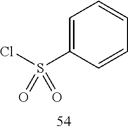 | 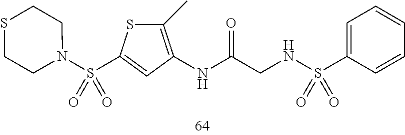 [SMILES: Cc1sc(S(=O)(=O)N2CCSCC2)cc1NC(=O)CNS(=O)(=O)c1ccccc1] | 27% |
| 2 | 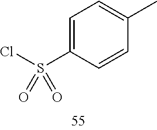 | 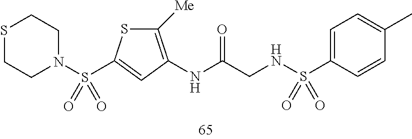 [SMILES: Cc1ccc(S(=O)(=O)NCC(=O)Nc2cc(S(=O)(=O)N3CCSCC3)sc2C)cc1] | 12% |
| 3 | 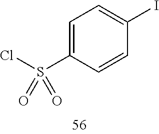 [SMILES: O=S(=O)(Cl)c1ccc(I)cc1] | 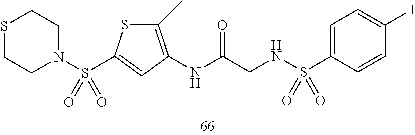 [SMILES: Cc1sc(S(=O)(=O)N2CCSCC2)cc1NC(=O)CNS(=O)(=O)c1ccc(I)cc1] | 27% |
| 4 | 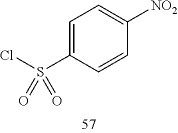 [SMILES: O=[N+]([O-])c1ccc(S(=O)(=O)Cl)cc1] | 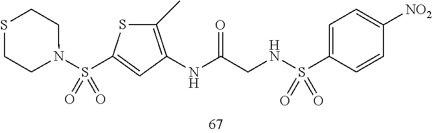 [SMILES: Cc1sc(S(=O)(=O)N2CCSCC2)cc1NC(=O)CNS(=O)(=O)c1ccc([N+](=O)[O-])cc1] | 20% |
| 5 | 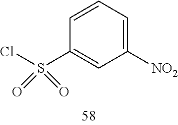 [SMILES: O=[N+]([O-])c1cccc(S(=O)(=O)Cl)c1] | 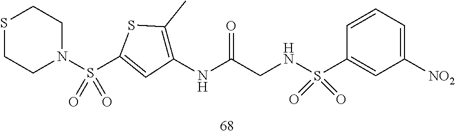 [SMILES: Cc1sc(S(=O)(=O)N2CCSCC2)cc1NC(=O)CNS(=O)(=O)c1cccc([N+](=O)[O-])c1] | 53% |
| 6 | 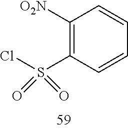 [SMILES: O=[N+]([O-])c1ccccc1S(=O)(=O)Cl] | 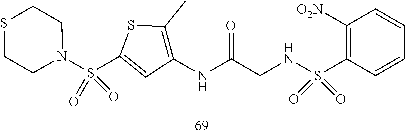 [SMILES: Cc1sc(S(=O)(=O)N2CCSCC2)cc1NC(=O)CNS(=O)(=O)c1ccccc1[N+](=O)[O-]] | 47% |
| 7 | 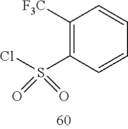 [SMILES: O=S(=O)(Cl)c1ccccc1C(F)(F)F] | 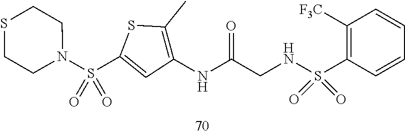 [SMILES: Cc1sc(S(=O)(=O)N2CCSCC2)cc1NC(=O)CNS(=O)(=O)c1ccccc1C(F)(F)F] | 24% |
| 8 | 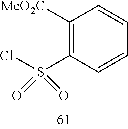 [SMILES: COC(=O)c1ccccc1S(=O)(=O)Cl] | 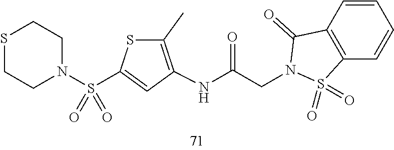 [SMILES: Cc1sc(S(=O)(=O)N2CCSCC2)cc1NC(=O)CN1C(=O)c2ccccc2S1(=O)=O] | 36% |
| 9 | 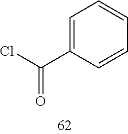 | 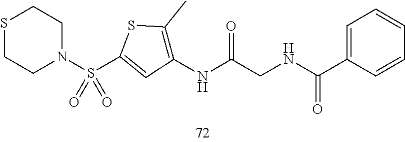 [SMILES: Cc1sc(S(=O)(=O)N2CCSCC2)cc1NC(=O)CNC(=O)c1ccccc1] | 31% |
| 10 | 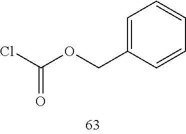 [SMILES: O=C(Cl)OCc1ccccc1] | 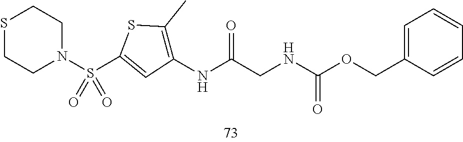 [SMILES: Cc1sc(S(=O)(=O)N2CCSCC2)cc1NC(=O)CNC(=O)OCc1ccccc1] | 31% |

The analogs with different aromatic spacer groups (section B) were also evaluated. Replacing the 2-methylthiophene with a 2-chlorothiophene gave analog 33 which was nearly equipotent as the parent K306 structure. Attempts were also made to replace the thiophene ring with a more stable benzene ring. This led to the synthesis of benzenesulfonamides 37 and 41. Activity was maintained with a phenyl ring when the sulfonamide and the amide were in a 1,4-orientation (compound 41), however the 1,3-orientation (compound 37) was less active.

Evaluation of K306 analogs with changes in section C was then undertaken. Analogs employing both enantiomers of alanine and phenylalanine were first investigated (48-51), with the results appearing to indicate that the presence of an(S) stereocenter led to compounds with better activator activity than the (R) enantiomers. Analogs bearing a phenylglycine and a proline in section C also showed good activator activity, with the proline sulfonamide 53 even demonstrating significant activity at 250 μM. Modification of the second sulfonamide section (section D) was then undertaken. Deletion of the fluorine (as in 64) or incorporation of a less polar group on the benzenesulfonamide (as in 65 and 66) led to a loss of bioactivity. Switching to the more polar nitro substituted sulfonamide 67 appeared to give an increase in the activity, so the 3-nitro and 2-nitro sulfonamides were also synthesized and evaluated. The 3-nitrobenzenesulfonamide analog 68 was disappointing, but the 2-nitrobenzenesulfonamide 69 was quite active. While the activity was promising, nitro groups can be problematic in the development of bioactive small molecules, as nitroreductases often will reduce the nitro group to an electrophilic nitroso derivative, which can have mutagenic effects and exhibit hepatotoxicity. While the nitro group in 69 was relatively hindered, this was still a concern, so some other bioisosteres for a nitro group were then evaluated beginning with the trifluoromethyl group in 70, which had good activator activity. An ester has also been used as a nitro bioisostere, but incorporation of the ester next to the sulfonamide led to a spontaneous cyclization to form the benzoic sulfimide 71. Sulfimide 71 displayed promising SHIP1 activator activity in the malachite green assay, nearly as potent as 69, demonstrating that a polar group in this position is beneficial for bioactivity. Other functional groups in lieu of the sulfonamide in section D (Table 2, entries 15-16) were prepared including the benzamide 72 and the carbamate 73, but these changes led to a loss of potency.

TABLE 4
Evaluation of K306 analogs in the Malachite Green Assay^{a}

| | | Analog Concentration | | |
|---|---|---|---|---|
| | | 500 | 250 | 125 |
| Entry | Bis-Sulfonamide | μM | μM | μM |
| 1 (K306) | 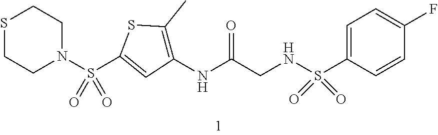 [SMILES: Cc1sc(S(=O)(=O)N2CCSCC2)cc1NC(=O)CNS(=O)(=O)c1ccc(F)cc1] | 133% | 106% | 95% |
| 2 (K381) | 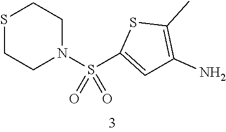 [SMILES: Cc1sc(S(=O)(=O)N2CCSCC2)cc1N] | 113% | 107% | 109% |
| 3 (K378) | 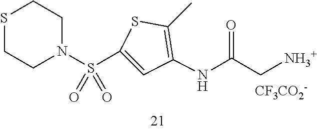 | 107% | 104% | 103% |
| 4 (K377) | 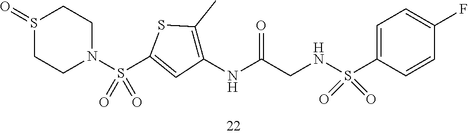 [SMILES: Cc1sc(S(=O)(=O)N2CCS(=O)CC2)cc1NC(=O)CNS(=O)(=O)c1ccc(F)cc1] | 80% | 89% | 98% |
| 5 | 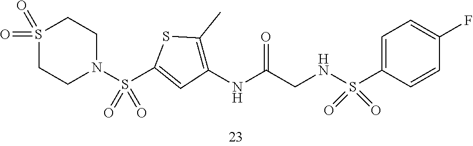 [SMILES: Cc1sc(S(=O)(=O)N2CCS(=O)(=O)CC2)cc1NC(=O)CNS(=O)(=O)c1ccc(F)cc1] | nd^{b} | nd^{b} | nd^{b} |
| 6 (K367) | 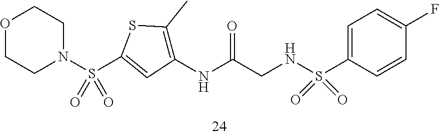 [SMILES: Cc1sc(S(=O)(=O)N2CCOCC2)cc1NC(=O)CNS(=O)(=O)c1ccc(F)cc1] | 94% | 93% | nd |
| 7 (K387) | 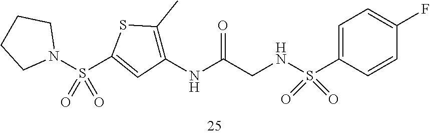 [SMILES: Cc1sc(S(=O)(=O)N2CCCC2)cc1NC(=O)CNS(=O)(=O)c1ccc(F)cc1] | 90% | 97% | 100% |
| 8 (K368) | 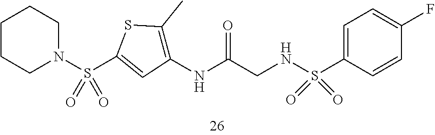 [SMILES: Cc1sc(S(=O)(=O)N2CCCCC2)cc1NC(=O)CNS(=O)(=O)c1ccc(F)cc1] | 128% | 119% | 116% |
| 9 (K388) | 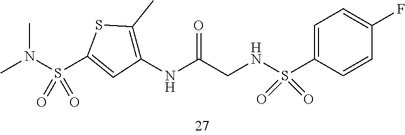 [SMILES: Cc1sc(S(=O)(=O)N(C)C)cc1NC(=O)CNS(=O)(=O)c1ccc(F)cc1] | 94% | 97% | 100% |
| 10 (K383) | 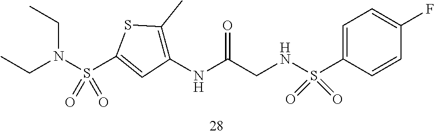 [SMILES: CCN(CC)S(=O)(=O)c1cc(NC(=O)CNS(=O)(=O)c2ccc(F)cc2)c(C)s1] | 109% | 93% | 101% |
| 11 (K382) | 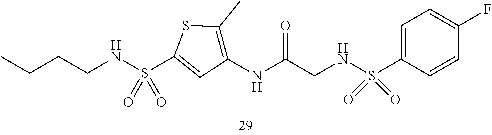 [SMILES: CCCCNS(=O)(=O)c1cc(NC(=O)CNS(=O)(=O)c2ccc(F)cc2)c(C)s1] | 122% | 124% | 124% |
| 12 (K401) | 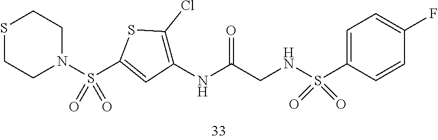 [SMILES: O=C(CNS(=O)(=O)c1ccc(F)cc1)Nc1cc(S(=O)(=O)N2CCSCC2)sc1Cl] | 125% | 109% | 102% |
| 13 (K400) | 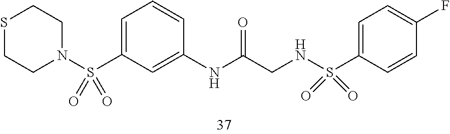 [SMILES: O=C(CNS(=O)(=O)c1ccc(F)cc1)Nc1cccc(S(=O)(=O)N2CCSCC2)c1] | 107% | 97% | 94% |
| 14 (K399) | 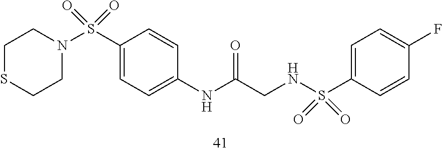 [SMILES: O=C(CNS(=O)(=O)c1ccc(F)cc1)Nc1ccc(S(=O)(=O)N2CCSCC2)cc1] | 130% | 105% | 112% |
| 15 (K389) | 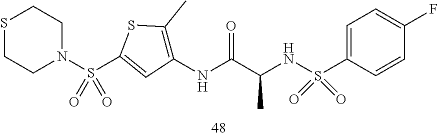 [SMILES: Cc1sc(S(=O)(=O)N2CCSCC2)cc1NC(=O)[C@H](C)NS(=O)(=O)c1ccc(F)cc1] | 132% | 118% | 89% |
| 16 (K386) | 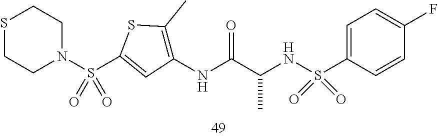 [SMILES: Cc1sc(S(=O)(=O)N2CCSCC2)cc1NC(=O)[C@@H](C)NS(=O)(=O)c1ccc(F)cc1] | 116% | 108% | nd |
| 17 (K396) | 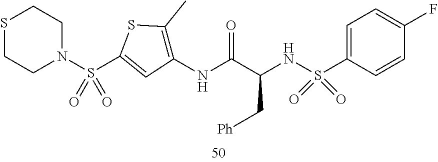 [SMILES: Cc1sc(S(=O)(=O)N2CCSCC2)cc1NC(=O)[C@H](Cc1ccccc1)NS(=O)(=O)c1ccc(F)cc1] | 130% | 74% | 71% |
| 18 (K395) | 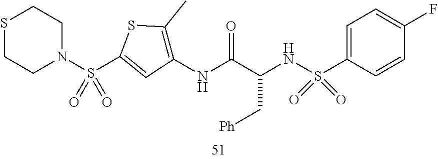 [SMILES: Cc1sc(S(=O)(=O)N2CCSCC2)cc1NC(=O)[C@@H](Cc1ccccc1)NS(=O)(=O)c1ccc(F)cc1] | 106% | 106% | 112% |
| 19 (K409) | 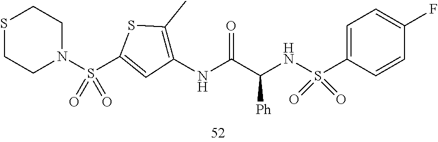 [SMILES: Cc1sc(S(=O)(=O)N2CCSCC2)cc1NC(=O)[C@@H](NS(=O)(=O)c1ccc(F)cc1)c1ccccc1] | 134% | 107% | 94% |
| 20 (K408) | 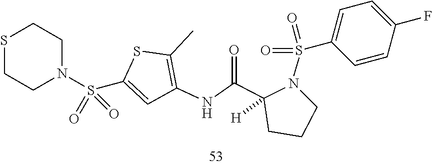 [SMILES: Cc1sc(S(=O)(=O)N2CCSCC2)cc1NC(=O)[C@@H]1CCCN1S(=O)(=O)c1ccc(F)cc1] | 130% | 127% | 107% |
| 21 (K376) | 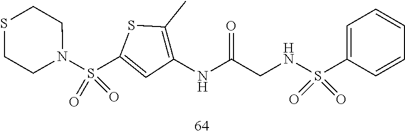 [SMILES: Cc1sc(S(=O)(=O)N2CCSCC2)cc1NC(=O)CNS(=O)(=O)c1ccccc1] | 107% | 106% | 104% |
| 22 (K369) | 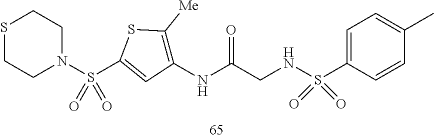 [SMILES: Cc1ccc(S(=O)(=O)NCC(=O)Nc2cc(S(=O)(=O)N3CCSCC3)sc2C)cc1] | 105% | 103% | 105% |
| 23 (K374) | 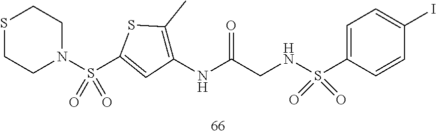 [SMILES: Cc1sc(S(=O)(=O)N2CCSCC2)cc1NC(=O)CNS(=O)(=O)c1ccc(I)cc1] | 119% | 83% | 99% |
| 24 (K375) | 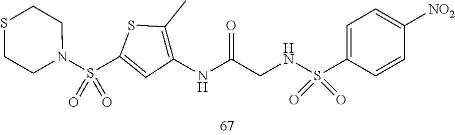 [SMILES: Cc1sc(S(=O)(=O)N2CCSCC2)cc1NC(=O)CNS(=O)(=O)c1ccc([N+](=O)[O-])cc1] | 150% | 115% | 108% |
| 25 (K392) | 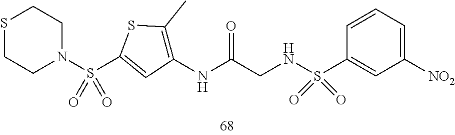 [SMILES: Cc1sc(S(=O)(=O)N2CCSCC2)cc1NC(=O)CNS(=O)(=O)c1cccc([N+](=O)[O-])c1] | 106% | 102% | nd |
| 26 (K391) | 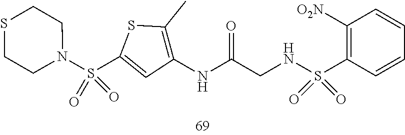 [SMILES: Cc1sc(S(=O)(=O)N2CCSCC2)cc1NC(=O)CNS(=O)(=O)c1ccccc1[N+](=O)[O-]] | 163% | 133% | 117% |
| 27 (K403) | 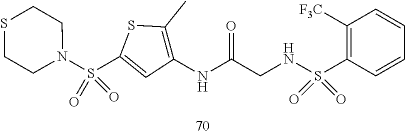 [SMILES: Cc1sc(S(=O)(=O)N2CCSCC2)cc1NC(=O)CNS(=O)(=O)c1ccccc1C(F)(F)F] | 139% | 108% | 106% |
| 28 (K402) | 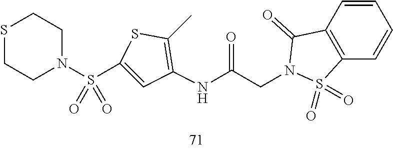 [SMILES: Cc1sc(S(=O)(=O)N2CCSCC2)cc1NC(=O)CN1C(=O)c2ccccc2S1(=O)=O] | 161% | 131% | 109% |
| 29 (K371) | 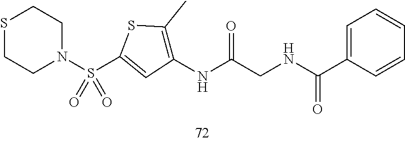 [SMILES: Cc1sc(S(=O)(=O)N2CCSCC2)cc1NC(=O)CNC(=O)c1ccccc1] | 83% | 94% | 97% |
| 30 (K373) | 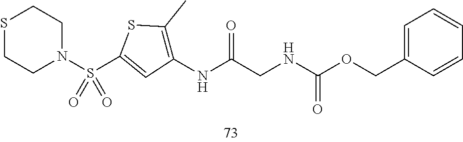 [SMILES: Cc1sc(S(=O)(=O)N2CCSCC2)cc1NC(=O)CNC(=O)OCc1ccccc1] | 105% | 102% | 93% |

^{a}Phosphatase activity for SHIP1 and SHIP2 enzyme as determined in the malachite green assay. Results were normalized to the phosphatase activity level from vehicle control (DMSO) used as 100% activity. Values >100% are attributed to activation, while values <100% show inhibition of the enzyme compared to the control. The assay was performed at the indicated concentration of the small molecule with 100 μM PI(3,4,5)P₃-diC8 as the substrate. ^{b}nd=not determined, compound 23 was insoluble.

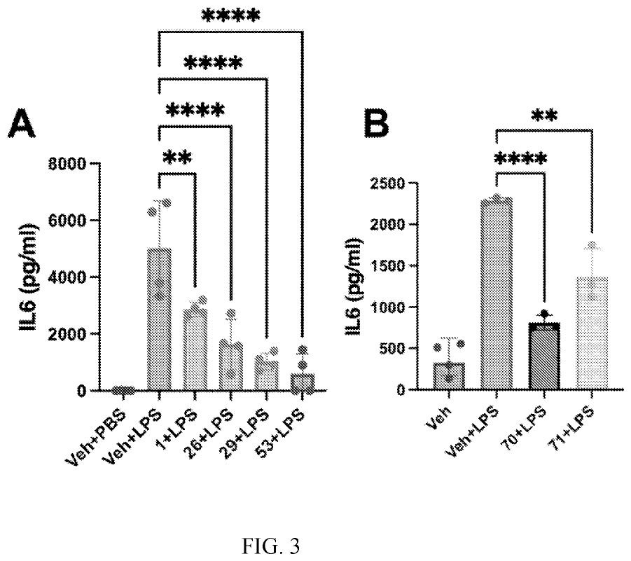

Having identified several new SHIP1 activators we then sought to determine if any of these molecules demonstrated anti-inflammatory activity in cells. While the malachite green assay is a rapid and inexpensive guide to identifying SHIP activators, we have found that it often requires a higher concentration of activator than is needed to observe effects in cell-based assays. Given this experience, some of the new activators were evaluated in a cell-based setting. Using the immortalized BV-2 cell line, which is derived from murine microglia and retains many microglial morphological and functional characteristics of microglia, an assay for the anti-inflammatory effects of these activators was developed. This assay involved treatment of the cells with lipopolysaccharides (LPS) to induce IL-6 production which can then be quantified with an ELISA. In the presence of K306 (1) the induction of IL-6 production by LPS is significantly diminished (FIG. 3A). Some of the bis-sulfonamide analogs that appeared active in the malachite assay and did not possess the oxidation prone thiomorpholine showed even greater potency than K306 (1) in this assay, with the piperidine sulfonamide 26 and the butyl sulfonamide 29 having especially significant reductions of IL-6 production (FIG. 3A). Modification of the amino acid section (section C) to a proline (compound 53) also gave a significant decrease in IL-6 formation in this assay. In a separate experiment the sector D analogs 70 and 71 (FIG. 3B) were also evaluated in this assay, with the 2-trifluoromethyl sulfonamide 70 demonstrating significant activity.

In summary, the K306 bis-sulfonamide structure is modified to determine structure activity relationships. The thiomorpholine subunit is shown to be a potential liability, as its oxidation leads to inactive or insoluble analogs. Replacement of the thiomorpholine with a piperidine or a butyl sulfonamide leads to structures that also act as SHIP1 activators without this liability. Substitution of the glycine for a proline in the amino acid sector of the molecule appears to provide a more potent activator that maintains its activity in cell-based assays of inflammation. Substitutions in the aromatic sector (sector B) show that there is some tolerance for change in this area, with a 1,4-disubstituted benzene ring being able to take the place of the thiophene. The 4-fluorobenzenesulfonamide can also be modified, although currently the most potent analogs have undesirable nitro groups associated with them. Studies on finding other polar groups that can serve as bioisosteres for the nitro group are in progress. Hybrid molecules containing the best structures from each sector are also under investigation. Studies on the development of new SHIP1 activators for the treatment of inflammatory disorders with increased potency, bioavailability, and stability will be reported in due course.

### EXAMPLES

### Structure Activity Studies on Bis-Sulfonamide SHIP1 Activators:

Cloning and expression of tSHIP1 of truncated human SHIP1 protein (tSHIP1) from human whole blood mRNA have been previously described (see Brooks R, Iyer, S., Akada, H., Neelam, S., Russo, C. M., Chisholm, J. D., and Kerr W. G. Coordinate Expansion of Murine Hematopoietic and Mesenchymal Stem Cell Compartments by SHIPi Stem Cells 2015; 33:848). Briefly, tSHIP1 (SHIP1, residues 397-864) was amplified from pET24TEV-tSHIP1. Plasmids were extracted with Midi Prep (Qiagen) and sequences were verified by sequencing of the entire expression regions. The plasmids were then transformed into E. coli BL21 (New England Biolabs). Fresh colonies were amplified in 1 L LB at 37° C., shaking at 250 rpm until OD600 nm reached 0.6. Protein expression was induced with 200 mM IPTG and incubation was continued at 16° C., shaking at 250 rpm for 16 h. Following centrifugation, bacteria pellets were stored overnight at −20° C. Protein was extracted form bacterial cell pellet using BugBuster HT (Millipore), according to manufacturer's recommendations. The His-tagged protein was purified by FPLC using HisTrapHP 5 ml column (Cytiva Life Sciences) at a 5 ml/min flow rate and 10 mM-1M imidazole gradient in Buffer A (20 mM Tris pH 8.0, 300 mM NaCl, 10 mM imidazole, 5 mM β-mercaptoethanol). Fractions containing active protein by Malachite Green Phosphatase Release Assay (Echelon, see below) were pooled, concentrated and buffer exchanged to reduce imidazole concentration by centrifugation using Pierce Protein Concentrators 30KMWCO PES, (ThermoFisher Scientific). Protein was aliquoted and stored at −80° C. in a mixture of Buffer A (without β-ME) and 50% glycerol.

Malachite Green Phosphatase Release Assays: Malachite Green Phosphatase Release Assays (Echelon Biosciences) were performed with recombinant human truncated SHIP1 (tSHIP1) (Brooks, 2015) and SHIP2 (Echelon Biosciences). Briefly, serial dilutions of the compounds dissolved in DMSO were added to the recombinant enzymes diluted in reaction buffer Rx (50 mM Hepes pH 7.4, 150 mM NaCl, 1 mM MgCl2, 0.25 mM EDTA) in triplicate reactions in 96-well plates. Reactions were incubated for 2 min at room temperature. 2.5 mL of 1 mM Phosphatidylinositol 3,4,5-trisphosphate diC8 (PI(3,4,5)P3diC8) (Echelon Biosciences) was added to each reaction to a final concentration of 100 μM in a final volume of 25 mL/well. Following 20 min incubation at 37° C., 100 μL of Malachite Green Solution (Echelon Biosciences) was added to each well and plates were incubated at room temperature in the dark for 15 min. Plates were then read at 620 nm on a plate reader (Synergy 2, BioTek).

Cell culture: The BV2 cell line was obtained from the ATCC (American Type Culture Collection). The ATCC and performs genetic validation on deposited cell lines. For the inflammatory cytokines production studies, BV2 cells were cultured in DMEM/F12 (Corning) 10% HI FBS at 37° C. and 5% CO2 in a humidified incubator and split 1:6 every 3 days with Cellstripper™ (Corning). Cells were kept at 37° C. and 5% CO2 in a humidified incubator.

ELISA measurements: Parental (scrambled guide control) BV2 cells were seeded at 5×105/mL, 250 mL/well in 48 well plate. Cell were treated with 10 mM small molecule or vehicle control (0.5% DMSO) and incubated for 1 h before addition of stimuli. LPS (10 ng/ml, E. coli O111: B4, Sigma) was added to the cells and incubated for 6 h to detect IL-6 production. Supernatants were spun down at 400×g for 5 minutes to remove cell debris and froze at −20° C. for ELISAs. IL-6 levels were quantified from cellular supernatants with ELISA MAX™ Standard Set Mouse IL-6 kit (Biolegend), following manufacturer's recommendations. We detected absorbance at 450 nm and removed 570 nm background absorbance, with a plate reader (Synergy 2, BioTek). All experiments were repeated three times and each value was reported as mean±SEM from biological replicates in the same assay. All statistical analysis were performed using GraphPad Prism (version 9.2). Two-way ANOVA was used to analyze all data with two independent variables as time and treatment or concentration and treatment, data is reported as Mean+/−SEM.

General Synthesis Experimental Information: All anhydrous reactions were run under a positive pressure of argon. DCM (DCM) was dried by passage through an alumina column. 1,2-Dichloroethane (DCE) was freshly distilled from calcium hydride before use. Tetrahydrofuran (THF) was freshly distilled from Na/benzophenone still before use. EA (EA) and hexanes were purchased from commercial sources and used as received. Silica gel column chromatography was performed using 60 Å silica gel (230-400 mesh). Melting points are uncorrected.

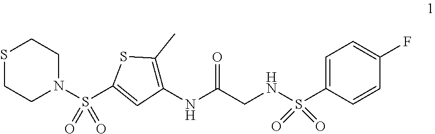 [SMILES: Cc1sc(S(=O)(=O)N2CCSCC2)cc1NC(=O)CNS(=O)(=O)c1ccc(F)cc1]

2-(4-Fluorobenzenesulfonamido)-N-[2-methyl-5-(thiomorpholine-4-sulfonyl)thiophen-3-yl]acetamide K306 (1). Amine salt 21 (0.600 g, 1.384 mmol) was dissolved in dry dichloromethane (2.15 mL) and dry TEA (0.425 mL, 3.045 mmol) and stirred at rt. 4-Fluorobenzenesulfonyl chloride (0.296 g, 1.523 mmol) was added and the mixture was stirred at rt for 24 h. The mixture was washed with water (3×5 mL) followed by a 5% HCl solution. The organics were collected, dried over MgSO4, filtered and concentrated. The crude mixture was purified by precipitation from dichloromethane yielding 1 as a white powder (0.213 g, 31%). (1). mp=177-180° C.; TLC Rf=0.371 (1% MeOH/DCM); IR (ATR) 3301, 3259, 2959, 2915, 1665, 1352, 1324, 1166, 1141 cm−1; 1H NMR (DMSO, 400 MHz) δ 7.82 (dd, J=8.5, 5.0 Hz, 2H), 7.60 (s, 1H), 7.36 (t, J=8.7 Hz, 2H), 3.63 (s, 2H), 3.19 (t, J=4.4 Hz, 4H), 2.68 (t, J=4.4 Hz, 4H), 2.31 (s, 3H); 13C NMR (DMSO, 100 MHz) δ 167.7, 163.8 (d, J=247.9 Hz), 138.5, 133.4, 133.2, 130.1, 130.0, 129.9 (d, J=9.2 Hz), 116.4 (J=22.5), 48.3, 46.5, 26.8, 12.8; Anal. Calcd for C17H20FN3O5S4: C, 41.37; H, 4.08; N, 8.51. Found: C, 41.66; H, 3.80; N, 8.87. HRMS calcd for C₁₇H₂₀FN₃O₅S₄K (M+K+): 531.9901. Found: 531.9903.

General procedure for the synthesis of sulfonamides 3-11: The amine (2 equiv) and 5-methyl-4-nitrothiophene-2-sulfonyl chloride (2) (1 equiv) were dissolved in 1,4-dioxane (1.0 M) and heated to 60° C. The mixture was stirred for 1 h at 60° C., after which the reaction was cooled to rt and 20 ml of water was added. The mixture was extracted with DCM (3×20 mL), and the organics were dried over MgSO4, filtered and concentrated. The crude mixture was purified via silica gel chromatography yielding the nitrothiophene intermediate. This nitrothiophene was dissolved in acetic acid (0.3 M) and iron power (5 equiv) was added. The mixture was heated to 60° C. and stirred for 1.5 h followed by removal of acetic acid in vacuo. The residue was taken up in EA and the iron powder was filtered away. The solution was washed with saturated NaHCO3 until a pH of 8 was reached. The organic layer was then dried over Na2SO4, filtered, and concentrated. The residue was purified via silica gel chromatography yielding the desired amine.

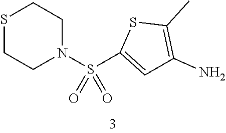 [SMILES: Cc1sc(S(=O)(=O)N2CCSCC2)cc1N]

2-Methyl-5-thiomorpholine-4-sulfonyl)thiophen-3-amine (3). Dark orange powder (0.230 g, 52%). mp=140-146° C.; TLC Rf=0.12 (30% EA/hexanes); IR (ATR) 3361, 2913, 2852, 1565, 1348, 1327, 1144, 898, 699 cm⁻¹; ¹H NMR (CDCl₃, 400 MHz) δ 7.54 (s, 1H), 3.72 (br s, 2H), 3.64 (t, J=4.6 Hz, 4H), 3.01 (t, J=4.8, 4H), 2.54 (s, 3H); ¹³C NMR (CDCl₃, 100 MHz) δ 140.0, 130.5, 125.6, 122.0, 48.0, 27.3, 11.8. Anal. Calcd for C₉H₁₄N₂O₂S₅: C, 38.83; H, 5.09; N, 10.06. Found: C, 39.17; H, 4.73; N, 9.84.

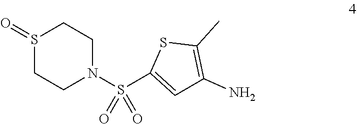 [SMILES: Cc1sc(S(=O)(=O)N2CCS(=O)CC2)cc1N]

4-[(4-Amino-5-methylthiophen-2-yl)sulfonyl]-1-thiomorpholin-1-one (4). Yellow solid (0.161 g, 99%). mp=151-154° C.; TLC Rf=0.54 (50% DCM/50% MeOH); IR (ATR) 3386, 3322, 3220, 2917, 2858, 1334, 1153, 1017 cm⁻¹; ¹H NMR (CD3OD, 400 MHz) δ 7.11 (s, 1H), 3.69-3.73 (m, 2H), 3.28-3.34 (m, 2H), 2.97-3.00 (m, 4H), 2.27 (s, 3H); 13C NMR (CD3OD, 100 MHz) 142.8, 128.7, 126.3, 120.7, 44.0, 36.7, 10.16; Anal. Calcd for C₉H₁₄N₂O₃S₃: C, 36.72; H, 4.79; N, 9.52. Found: C, 36.43; H, 4.73; N, 9.32.I

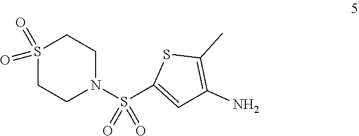 [SMILES: Cc1sc(S(=O)(=O)N2CCS(=O)(=O)CC2)cc1N]

4-[(4-Amino-5-methylthiophen-2-yl)sulfonyl]-1-thiomorpholine-1,1-dione (5). Yellow solid (0.237, 19%). mp=194-199° C.; TLC Rf=0.11 (50% EA/hexanes); IR (ATR) 3448, 3371, 3071, 3003, 2930, 2852, 1627, 1561, 1351, 1296, 1124, 1115 cm−1; 1H NMR (CD3OD, 400 MHz) δ 7.13 (s, 1H), 3.59-3.61 (m, 4H), 3.22 (t, J=5.4 Hz, 4H), 2.29 (s, 3H); ¹³C NMR (CD3OD, 100 MHz) 142.8, 129.3, 126.4, 121.0, 50.4, 45.1, 10.2; Anal. Calcd for C9H14N2O4S3: C, 34.83; H, 4.55; N, 9.03. Found: C, 34.99; H, 4.19; N, 8.86.

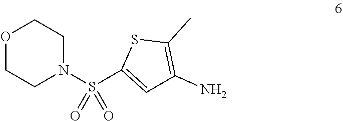 [SMILES: Cc1sc(S(=O)(=O)N2CCOCC2)cc1N]

2-Methyl-5-(morpholine-4-sulfonyl)thiophen-3-amine (6). Orange solid (0.80 g, 89%). TLC Rf=0.22 (3% MeOH/CHCl₃); ¹H NMR (CDCl₃, 400 MHz) δ 7.00 (s, 1H), 3.76 (t, J=4.7 Hz 4H), 3.45 (bs, 2H), 3.04 (t, J=4.8 Hz, 4H), 2.27 (s, 3H); ¹³C NMR (CDCl₃, 100 MHz) 141.2, 128.5, 126.0, 121.1, 66.1, 46.0, 11.7.

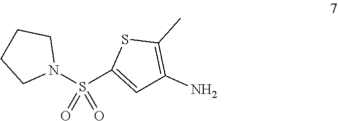

2-Methyl-5-(pyrrolidine-1-sulfonyl)thiophen-3-amine (7). Tan solid (0.41 g, 58%). mp=152-155° C.; TLC Rf=0.22 (50% EA/hexanes); IR (ATR) 3452, 3367, 2980, 2863, 1627, 1569, 1326, 1144 cm⁻¹; ¹H NMR (CD₃OD, 400 MHz) δ 7.09 (s, 3H), 3.24 (t, J=6.6 Hz, 4H), 2.27 (s, 3H), 1.77 (pent, J=6.8 Hz, 4H); ¹³C NMR (CDCl₃, 100 MHz) 136.2, 131.4, 129.2, 126.0, 48.2, 25.4, 12.1; Anal. Calcd for C₉H₁₄N₂O₂S2: C, 43.88; H, 5.73; N, 11.34. Found: C, 43.60; H, 5.36; N, 11.20.

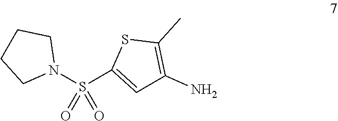

2-Methyl-5-(piperidine-1-sulfonyl)thiophen-3-amine (8). Tan solid (0.63 g, 70%). mp=96-101° C.; TLC Rf=0.30 (50% EA/hexanes); IR (ATR) 3426, 3349, 2979, 2949, 2851, 1618, 1566, 1543, 1334, 1322, 572 cm⁻¹; ¹H NMR (CDCl₃, 400 MHz) δ 7.02 (s, 1H), 3.75 (bs, 2H), 3.01 (t, J=5.5 Hz, 4H), 2.29 (s, 3H), 1.67 (quin, J=5.8 Hz, 4H), 1.45 (p, J=6.0 Hz, 2H); ¹³C NMR (CDCl₃, 100 MHz) 139.5, 130.3, 125.6, 121.9, 47.0, 25.1, 23.5, 11.8; Anal. Calcd for C₁₀H₁₆N₂O₂S₂: C, 46.13; H, 6.19; N, 10.76. Found: C, 46.00; H, 6.45; N, 10.66.

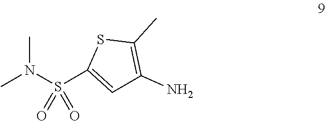

4-Amino-N,N,5-trimethylthiophene-2-sulfonamide (9). Yellow solid (0.240 g, 39%). mp=144-146° C.; TLC Rf=0.46 (50% EA/hexanes); IR (ATR) 3454, 3372, 3236, 3063, 2975, 2908, 1630, 1563, 1324, 1138 cm⁻¹; ¹H NMR (CD3OD, 400 MHz) δ 7.06 (s, 1H), 2.69 (s, 6H), 2.27 (s, 3H); ¹³C NMR (CD₃OD, 100 MHz) 142.3, 127.8, 126.2, 120.1, 37.0, 10.1; Anal. Calcd for C₇H₁₂N₂O₂S₂: C, 38.16; H, 5.49; N, 12.72. Found: C, 38.09; H, 5.76; N, 12.85.

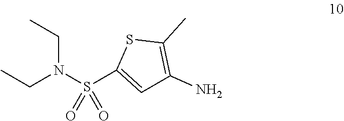

4-Amino-N,N-diethyl-5-methylthiophene-2-sulfonamide (10). Orange solid (0.150 g, 30%). mp=72-74° C.; TLC Rf=0.26 (50% EA/hexanes); IR (ATR) 3434, 3356, 2982, 2937, 1320, 1133 cm⁻¹; ¹H NMR (CDCl₃, 400 MHz) δ 6.98 (s, 1H), 3.52 (bs, 2H), 3.16 (q, J=7.2 Hz, 4H), 2.20 (s, 3H), 1.13 (t, J=7.2 Hz, 6H); ¹³C NMR (CDCl₃, 100 MHz) 140.8, 133.7, 125.1, 119.5, 42.7, 14.3, 11.6; Anal. Ca lcd for C₉H₁₆N₂O₂S₂: C, 43.53; H, 6.49; N, 11.28. Found: C, 43.18; H, 6.19; N, 11.48.

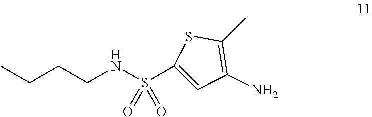

4-Amino-N-butyl-5-methylthiophene-2-sulfonamide (11). Off-white solid (0.384 g, 51%). mp=65-67° C.; TLC Rf=0.32 (50% EA/hexanes); IR (ATR) 3371, 3313, 3094, 2967, 2869, 1449, 1316, 1148, 1135 cm⁻¹; ¹H NMR (CDCl₃, 400 MHz) δ 7.06 (s, 1H), 4.86 (t, J=5.9 Hz, 1H), 3.65 (bs, 2H), 2.96 (q, J=6.8 Hz, 2H), 2.23 (s, 3H), 1.45 (p, J=7.1 Hz, 2H), 1.29 (sextet, J=7.2 Hz, 2H), 0.86 (t, J=7.4 Hz, 3H); ¹³C NMR (CDCl₃, 100 MHz) 140.8, 133.9, 125.6, 120.3, 43.2, 31.4, 19.8, 13.6, 11.7; Anal. Calcd for C₉H₁₆N₂O₂S₂: C, 43.53; H, 6.49; N, 11.28. Found: C, 43.63; H, 6.79; N, 11.04.

General procedure for the synthesis of amides 12-20: The desired boc-protected amino acid (1.2 equiv), thiophenylamine (1 equiv), HATU (2 equiv) and DIPEA (2 equiv) were dissolved in dry DMF (0.08 M) under argon. The mixture was stirred for 24 h at rt. The reaction was diluted with EA, washed with NH₄Cl (3×15 mL) and brine. The organic layer was then separated, dried over MgSO₄, filtered and concentrated. The residue was purified via silica gel chromatography yielding the desired amide product.

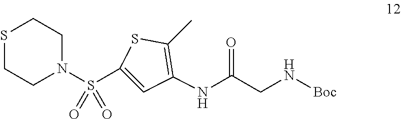 [SMILES: Cc1sc(S(=O)(=O)N2CCSCC2)cc1NC(=O)CNC(=O)OC(C)(C)C]

tert-Butyl N-(([2-methyl-5-(thiomorpholine-4-sulfonyl)thiophen-3-yl]carbamoyl)methyl) carbamate (12). Yellow oil (0.250 g, 76%). TLC Rf=0.308 (50% EA/hexanes); IR (ATR) 3656, 3254, 2980, 2971, 1665, 1350, 1154, 1141 cm-1; 1H NMR (CDCl₃, 400 MHz) δ 8.55 (br s, 1H), 7.78 (s, 1H), 5.60 (t, J=5.7 Hz, 1H), 3.89 (d, J=5.6 Hz, 2H), 3.32 (t, J=4.0 Hz, 4H), 2.68 (t, J=5.2 Hz, 4H), 2.32 (s, 3H), 1.43 (s, 9H); ¹³C NMR (CDCl₃, 100 MHz) δ 168.0, 156.9, 132.8, 132.2, 130.9, 128.6, 80.9, 47.9, 45.1, 28.3, 27.2, 12.4.

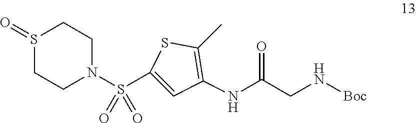 [SMILES: Cc1sc(S(=O)(=O)N2CCS(=O)CC2)cc1NC(=O)CNC(=O)OC(C)(C)C]

tert-Butyl-N-[({2-methyl-5-[(1-oxo-1-thiomorpholin-4-yl)sulfonyl]thiophen-3-yl}carbamoyl)methyl] carbamate (13). Clear oil (0.200 g, 73%). TLC Rf=0.17 (100% EA); IR (ATR) 3283, 2979, 2929, 2861, 1683, 1150, 1021 cm⁻¹; ¹H NMR (CDCl₃, 400 MHz) δ 8.55 (bs, 1H), 8.04 (s, 1H), 5.25 (bs, 1H), 3.89 (d, J=4.5 Hz, 2H), 3.80 (d, J=13.3 Hz, 2H), 3.45 (t, J=11.5 Hz, 2H), 2.94 (d, J=13.9 Hz, 2H), 2.79-2.86 (m, 2H), 2.38 (s, 3H), 1.49 (s, 9H); ¹³C NMR (CD3OD, 100 MHz) 171.2, 169.6, 136.0, 132.5, 130.3, 130.1, 79.4, 44.0, 43.3, 36.7, 27.3, 11.2; Anal. Calcd for C16H25N3O6S3: C, 42.56; H, 5.58; N, 9.34. Found: C, 42.28; H, 5.63; N, 9.70.

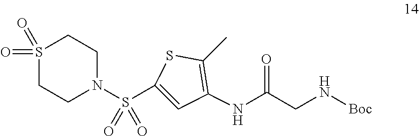 [SMILES: Cc1sc(S(=O)(=O)N2CCS(=O)(=O)CC2)cc1NC(=O)CNC(=O)OC(C)(C)C]

tert-Butyl-N-[({5-[(1,1-dioxo-1-thiomorpholine-4-yl)sulfonyl]-2-methylthiophen-3-yl}carbamoyl)methyl] carbamate (14). Light-yellow powder (0.140 g, 39%). mp=187-190° C.; TLC Rf=0.51 (30% EA/hexanes); IR (ATR) 3273, 2987, 2928, 2395, 1689, 1670, 1308, 1150, 1125 cm⁻¹; ¹H NMR (CD3OD, 400 MHz) δ 7.74 (s, 1H), 3.88 (s, 2H), 3.65 (bs, 4H), 3.23 (t, J=5.4 Hz, 4H), 2.42 (s, 3H), 1.47 (s, 9H); 13C NMR (CD3OD, 100 MHz) 169.8, 157.2, 136.5, 132.5, 130.8, 130.4, 79.4, 50.5, 45.1, 43.3, 27.3, 11.2; Anal. Calcd for C16H25N3O7S3: C, 41.10; H, 5.39; N, 8.99. Found: C, 39.89; H, 5.30; N, 9.34.

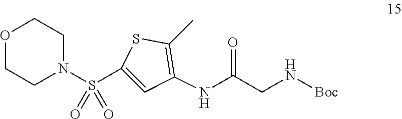 [SMILES: Cc1sc(S(=O)(=O)N2CCOCC2)cc1NC(=O)CNC(=O)OC(C)(C)C]

tert-Butyl-N-[({2-methyl-5-(morpholine-4-sulfonyl)thiophen-3-yl}carbamoyl)methyl] carbamate (15). Off-white solid (0.370 g, 86%). mp=124-128° C.; TLC Rf=0.21 (50% EA/hexanes); IR (ATR) 3623, 3313, 2976, 2924, 2859, 1681, 1504, 1348, 1152, 941 cm⁻¹; ¹H NMR (CDCl₃, 400 MHz) δ 8.48 (bs, 1H), 7.92 (s, 1H), 5.27 (bs, 1H), 3.91 (d, J=4.2 Hz, 2H), 3.77 (t, J=4.2 Hz, 4H), 3.06 (t, J=4.2 Hz, 4H), 2.38 (s, 3H), 1.49 (s, 9H); 13C NMR (CDCl₃, 100 MHz) 167.9, 156.8, 132.7, 132.1, 130.8, 128.5, 80.8, 47.8, 45.0, 28.2, 27.1, 12.3; Anal. Calcd for C16H25N3O6S2: C, 45.81; H, 6.01; N, 10.02. Found: C, 45.61; H, 6.04; N, 9.73.

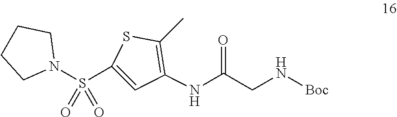 [SMILES: Cc1sc(S(=O)(=O)N2CCCC2)cc1NC(=O)CNC(=O)OC(C)(C)C]

tert-Butyl-N-[({2-methyl-5-(pyrrolidine-1-sulfonyl)thiophen-3-yl}carbamoyl)methyl] carbamate (16). Tan solid (0.280 g, 64%). mp=63-67° C.; TLC Rf=0.24 (50% EA/hexanes); IR (ATR) 3317, 2975, 2930, 2873, 1679, 1503, 1344, 1147 cm⁻¹; ¹H NMR (CD3OD, 400 MHz) δ 7.67 (s, 1H), 3.87 (s, 2H), 3.24-3.28 (m, 4H), 2.39 (s, 3H), 1.77-1.80 (m, 4H), 1.47 (s, 9H); 13C NMR (CDCl₃, 100 MHz) 171.1, 158.6, 136.0, 135.8, 133.2, 130.3, 80.8, 44.7, 44.0, 28.7, 14.8, 12.5; Anal. Calcd for C16H25N3O5S2: C, 47.63; H, 6.25; N, 10.41. Found: C, 47.30; H, 5.93; N, 10.14.

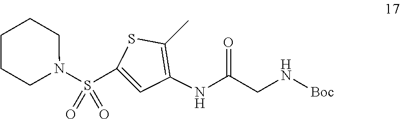 [SMILES: Cc1sc(S(=O)(=O)N2CCCCC2)cc1NC(=O)CNC(=O)OC(C)(C)C]

tert-butyl-N-[({2-methyl-5-(piperidine-1-sulfonyl)thiophen-3-yl}carbamoyl)methyl] carbamate (17). Yellow solid (0.220 g, 47%). mp=156-160° C.; TLC Rf=0.30 (50% EA/hexanes); IR (ATR) 3321, 2935, 2856, 1685, 1676, 1159, 1142 cm-1; 1H NMR (acetone-d6, 400 MHz) δ 9.01 (s, 1H), 7.82 (s, 1H), 6.37 (s, 1H), 3.90 (d, J=5.8 Hz 2H), 3.00 (t, J=5.4 Hz, 4H), 2.40 (s, 3H), 1.65 (p, J=5.8 Hz, 4H), 1.44-1.49 (m, 11H); ¹³C NMR (acetone-d6, 100 MHz) 167.8, 156.3, 133.1, 132.0, 130.5, 128.8, 78.7, 46.9, 44.1, 27.7, 25.0, 23.1, 11.6; Anal. Calcd for C17H27N3O5S2: C, 48.90; H, 6.52; N, 10.06. Found: C, 48.63; H, 6.29; N, 10.03.

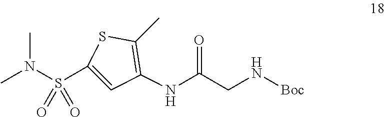 [SMILES: Cc1sc(S(=O)(=O)N(C)C)cc1NC(=O)CNC(=O)OC(C)(C)C]

tert-Butyl-N-[({5-(dimethylsulfamoyl)-2-methylthiophen-3-yl}carbamoyl)methyl] carbamate (18). Orange powder (0.230 g, 67%). mp=88-93° C.; TLC Rf=0.20 (50% EA/hexanes); IR (ATR) 3314, 2974, 2928, 1677, 1502, 1341, 1142 cm⁻¹; ¹H NMR (CD3OD, 400 MHz) δ 7.64 (s, 1H), 3.87 (bs, 2H), 2.72 (s, 6H), 2.40 (s, 3H), 1.47 (s, 9H); ¹³C NMR (CD3OD, 100 MHz) 169.7, 165.3, 135.6, 132.2, 129.8, 129.3, 78.1, 43.3, 36.9, 27.3, 11.2; Anal. Calcd for C14H23N3O5S2: C, 44.55; H, 6.14; N, 11.13. Found: C, 44.67; H, 6.31; N, 10.88.

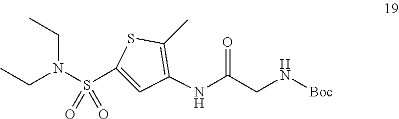 [SMILES: CCN(CC)S(=O)(=O)c1cc(NC(=O)CNC(=O)OC(C)(C)C)c(C)s1]

tert-Butyl-N-[({5-(diethylsulfamoyl)-2-methylthiophen-3-yl}carbamoyl)methyl] carbamate (19). The crude oil was purified via silica gel column chromatography using a 50% EA/hexanes eluent yielding an orange solid (0.120 g, 49%). mp=47-49° C.; TLC Rf=0.31 (50% EA/hexanes); IR (ATR) 3309, 2978, 2935, 1679, 1501, 1140, 1015 cm⁻¹; ¹H NMR (CD3OD, 400 MHz) δ 7.64 (s, 1H), 3.86 (bs, 2H), 3.22 (q, J=7.4 Hz, 4H), 2.38 (s, 3H), 1.47 (s, 9H), 1.17 (t, J=7.2 Hz, 6H); 13C NMR (CD3OD, 100 MHz) 169.7, 157.2, 134.6, 134.4, 131.8, 128.9, 79.4, 43.3, 42.6, 27.3, 13.4, 11.1; Anal. Calcd for C16H27N3O5S2: C, 47.39; H, 6.71; N, 10.36. Found: C, 47.58; H, 6.42; N, 10.34.

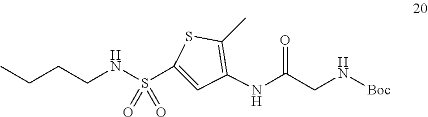 [SMILES: CCCCNS(=O)(=O)c1cc(NC(=O)CNC(=O)OC(C)(C)C)c(C)s1]

tert-Butyl-N-[({5-(butylsulfamoyl)-2-methylthiophen-3-yl}carbamoyl)methyl] carbamate (20). Off-white foam (0.320 g, 70%). mp=65-67° C.; TLC Rf=0.33 (50% EA/hexanes); IR (ATR) 3657, 3217, 2980, 1676, 1505, 1146 cm-1; 1H NMR (CDCl₃, 400 MHz) δ 8.44 (bs, 1H), 7.83 (bs, 1H), 7.25 (s, 1H), 5.44 (bs, 1H), 3.91 (s, 2H), 3.01 (t, J=6.6 Hz, 2H), 2.33 (s, 3H), 1.43-1.53 (m, 11H), 1.32 (sext, J=7.0 Hz, 2H), 0.88 (t, J=7.3 Hz, 3H); 13C NMR (CDCl3, 100 MHz) 167.9, 156.9, 135.2, 131.7, 128.0, 128.0, 81.0, 45.3, 43.3, 31.5, 28.3, 19.7, 13.6, 12.4; Anal. Calcd for C9H27N3O5S2: C, 47.39; H, 6.71; N, 10.36. Found: C, 47.43; H, 6.40; N, 10.49.

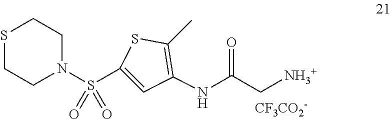

2-Aminotrifluoroacetate-N-[2-methyl-5-(thiomorpholine-4-sulfonyl)thiophene-3-yl]acetamide (21). Boc protected amine 12 was dissolved in TFA and stirred at rt for 0.5 h. The solvent was then removed in vacuo to provide 21 as an off-white solid was recovered (1.57 g, 96%). mp=190° C. (dec); IR (ATR) 3255, 2980, 2915, 1665, 1350, 1329, 1153 cm⁻¹; ¹H NMR (CD3OD, 400 MHz) δ 7.79 (s, 1H), 3.93 (s, 2H), 3.37 (t, J=5.0 Hz, 4H), 2.74 (t, J=5.2, 4H), 2.45 (s, 3H); ¹³C NMR (CD3OD, 100 MHz) δ 164.5, 138.9, 134.7, 131.7, 131.4, 128.9, 40.4, 26.7, 11.1. Anal. Calcd for C₁₃H₁₈F₃N₃O₅S₃: C, 34.74; H, 4.04; N, 9.35. Found: C, 34.97; H, 4.16; N, 8.96.

### General Procedure for the Synthesis of Sulfonamides 22-29.

The Boc protected amine was dissolved in TFA (0.5 M) and stirred at rt for 0.5 h. The solvent was then removed in vacuo to provide the amine TFA salt. This amine salt was dissolved in dry DCM (0.7 M) and dry TEA was added (2.2 equiv). The desired sulfonyl chloride or other electrophile (1.1 equiv) was then added and the mixture was stirred at rt for 24 h under argon. The reaction was then taken up in EA, washed with H₂O and 5% HCl. The organic layer was then separated, dried over Na₂SO₄, filtered, and concentrated. The residue was then purified by trituration from DCM or by silica gel chromatography yielding the pure product.

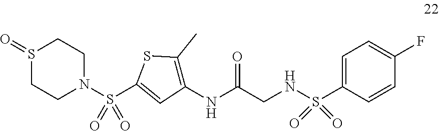 [SMILES: Cc1sc(S(=O)(=O)N2CCS(=O)CC2)cc1NC(=O)CNS(=O)(=O)c1ccc(F)cc1]

2-(4-Fluorobenzenesulfonamido)-N-{2-methyl-5-[1-oxo-1-thiomorpholin-4-yl)sulfonyl] thiophen-3-yl}acetamide (22). The crude oil was purified via trituration from DCM yielding a white solid (0.021 g, 13%). mp=210-212° C.; TLC R_{f}=0.49 (95% DCM/5% MeOH); IR (ATR) 3360, 3267, 3106, 2927, 2858, 1685, 1670, 1352, 1328, 1154 cm⁻¹; ¹H NMR (DMSO-d₆, 400 MHz) δ 9.77 (s, 1H), 8.17 (bs, 1H), 7.89 (m, 2H), 7.65 (s, 1H), 7.44 (t, J=8.4 Hz, 2H), 3.73 (d, J=4.8 Hz, 2H), 3.60 (d, J=12.4 Hz, 2H), 3.05-3.10 (m, 2H), 2.90-2.99 (m, 4H), 2.35 (s, 3H); ¹³C NMR (DMSO-d₆, 100 MHz) 166.8, 161.6 (d, J=364 Hz), 137.2 (d, J=3.0 Hz), 134.5, 133.3, 130.1 (d, J=9.7 Hz), 129.9, 129.2, 116.7 (d, J=23.2 Hz), 45.6, 43.9, 36.9, 12.9; Anal. Calcd for C₁₇H₂₀FN₃O₆S₄: C, 40.07; H, 3.96; N, 8.25. Found: C, 39.93; H, 4.24; N, 8.08.

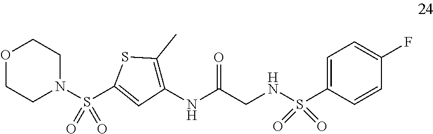 [SMILES: Cc1sc(S(=O)(=O)N2CCOCC2)cc1NC(=O)CNS(=O)(=O)c1ccc(F)cc1]

2-(4-Fluorobenzenesulfonamido)-N-[2-methyl-5-(morpholine-4-sulfonyl)thiophen-3-yl]acetamide (24). White powder (0.104 g, 30%). mp=173-177° C.; TLC R_{f}=0.28 (50% EA/hexanes); IR (ATR) 3650, 3395, 3233, 2980, 2890, 2856, 1673, 1345, 1153 cm⁻¹; ¹H NMR (CD3OD, 400 MHz) δ 8.19 (s, 1H), 7.91-7.94 (m, 2H), 7.77 (s, 1H), 7.23-7.28 (m, 2H), 5.45 (t, J=5.9 Hz, 1H), 3.72-3.78 (m, 6H), 3.02-3.06 (m, 4H), 2.40 (s, 3H); ¹³C NMR (CD3OD, 100 MHz) 165.6 (d, J=255.0 Hz), 165.5, 134.2, 133.9, 131.5, 130.1 (d, J=9.9 Hz), 129.8, 128.8, 116.8 (d, J=23.1 Hz), 66.0, 46.3, 46.0, 12.5; Anal. Calcd for C₁₇H₂₀FN₃O₆S₃: C, 42.76; H, 4.22; N, 8.80. Found: C, 42.82; H, 4.24; N, 8.52.

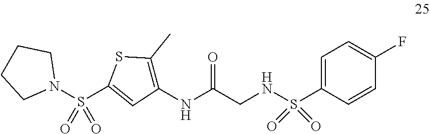 [SMILES: Cc1sc(S(=O)(=O)N2CCCC2)cc1NC(=O)CNS(=O)(=O)c1ccc(F)cc1]

2-(4-Fluorobenzenesulfonamido)-N-[2-methyl-5-(pyrrolidine-1-sulfonyl)thiophen-3-yl]acetamide (25). White foam (0.020 g, 10%). mp=159-163° C.; TLC R_{f}=0.18 (50% EA/hexanes); IR (ATR) 3360, 3239, 3099, 2885, 1676, 1508, 1328, 1151 cm⁻¹; ¹H NMR (CD3OD, 400 MHz) δ 7.94-7.97 (m, 2H), 7.55 (s, 1H), 7.31 (t, J=8.5 Hz, 2H), 3.77 (s, 2H), 3.25 (bs, 3H), 2.35 (s, 3H), 1.79 (bs, 4H); ¹³C NMR (CD₃OD, 100 MHz) 167.8, 165.2 (d, J=251.6), 136.2 (d, J=2.7 Hz), 135.2, 131.7, 130.7, 129.9 (d, J=9.5 Hz), 129.3, 115.8 (d, J=23.4 Hz), 47.8, 45.1, 24.9, 11.1; Anal. Calcd for C₁₇H₂₀FN₃O₅S₃: C, 44.24; H, 4.37; N, 9.10. Found: C, 44.21; H, 4.44; N, 8.97.

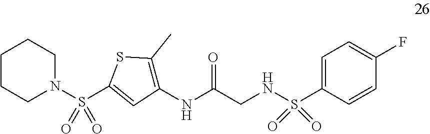 [SMILES: Cc1sc(S(=O)(=O)N2CCCCC2)cc1NC(=O)CNS(=O)(=O)c1ccc(F)cc1]

2-(4-Fluorobenzenesulfonamido)-N-[2-methyl-5-(piperidine-1-sulfonyl)thiophen-3-yl]acetamide (26). The crude oil was purified via silica gel column chromatography using a 50% EA/hexanes eluent yielding a white solid (0.092 g, 37%). mp=79-84° C.; TLC R_{f}=0.21 (50% EA/hexanes); IR (ATR) 3300, 2939, 2853, 1686, 1589, 1335, 1154, 1143 cm⁻¹; ¹H NMR (CD3OD, 400 MHz) δ 7.95 (m, 2H), 7.48 (s, 1H), 7.31 (t, J=8.7 Hz, 2H), 3.77 (s, 2H), 3.00 (t, J=5.2 Hz, 4H), 2.35 (s, 3H), 1.62-1.69 (m, 4H), 1.47-1.50 (m, 2H); ¹³C NMR (CD3OD, 100 MHz) 166.7, 164.6 (d, J=250.4 Hz), 137.3 (d, J=2.9 Hz), 133.5, 133.1, 130.1 (d, J=9.4 Hz), 129.7, 129.4, 116.6 (d, J=23.1 Hz), 47.1, 45.6, 25.0, 23.2, 12.9; Anal. Calcd for C₁₈H₂₂FN₃O₅S₃: C, 45.46; H, 4.66; N, 8.84. Found: C, 45.47; H, 4.92; N, 8.99.

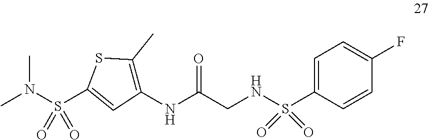 [SMILES: Cc1sc(S(=O)(=O)N(C)C)cc1NC(=O)CNS(=O)(=O)c1ccc(F)cc1]

N-[5-(Dimethylsulfamoyl)-2-methylthiophen-3-yl]-2-(4-fluorobenzenesulfonamido) acetamide (27). White powder (0.120 g, 51%). mp=123-128° C.; TLC R_{f}=0.21 (50% EA/hexanes); IR (ATR) 3321, 3278, 3107, 2928, 1658, 1590, 1342, 1155, 1137 cm⁻¹; ¹H NMR (CD3OD, 400 MHz) δ 7.93-7.97 (m, 2H), 7.52 (s, 1H), 7.31 (t, J=8.1 Hz, 2H), 3.77 (s, 2H), 2.71 (s, 6H), 2.36 (s, 3H); ¹³C NMR (CD₃OD, 100 MHz) 167.8, 165.2 (d, J=251.5), 136.2 (d, J=3.1 Hz), 135.6, 131.8, 129.9 (d, J=9.4 Hz), 129.6, 129.4, 115.8 (d, J=24.3 Hz), 45.1, 36.9, 11.1; Anal. Calcd for C₁₅H₁₈FN₃O₅S₃: C, 41.37; H, 4.17; N, 9.65. Found: C, 41.24; H, 4.28; N, 9.36.

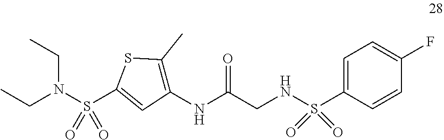 [SMILES: CCN(CC)S(=O)(=O)c1cc(NC(=O)CNS(=O)(=O)c2ccc(F)cc2)c(C)s1]

N-[5-(Diethylsulfamoyl)-2-methylthiophen-3-yl]-2-(4-fluorobenzenesulfonamido) acetamide (28). Orange wax (0.108 g, 80%). mp=44-48° C.; TLC R_{f}=0.18 (50% EA/hexanes); IR (ATR) 3309, 2977, 2934, 2360, 2343, 1685, 1590, 1494, 1327, 1141 cm⁻¹; ¹H NMR (CD₃OD, 400 MHz) δ 7.95 (m, 2H), 7.52 (s, 1H), 7.30 (t, J=8.6 Hz, 2H), 3.76 (s, 2H), 3.20 (q, J=7.2 Hz, 4H), 2.33 (s, 3H), 1.16 (t, J=7.2 Hz, 6H); ¹³C NMR (CD₃OD, 100 MHz) 167.7, 165.0 (d, J=250.1 Hz), 136.2 (d, J=2.5 Hz), 134.6, 134.5, 131.5, 129.9 (d, J=9.4 Hz), 128.6, 115.8 (d, J=23.8 Hz), 45.1, 42.6, 13.4, 11.1; Anal. Calcd for C₁₇H₂₂FN₃O₅S₃: C, 44.05; H, 4.78; N, 9.06. Found: C, 44.12; H, 4.99; N, 8.79.

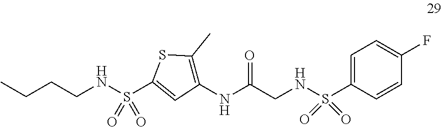 [SMILES: CCCCNS(=O)(=O)c1cc(NC(=O)CNS(=O)(=O)c2ccc(F)cc2)c(C)s1]

N-[5-(Butylsulfamoyl)-2-methylthiophen-3-yl]-2-(4-fluorobenzenesulfonamido) acetamide (29). White powder (0.185 g, 13%). mp=161-164° C.; TLC R_{f}=0.19 (50% EA/50% hexane); IR (ATR) 3247, 2964, 2934, 2876, 1652, 1325, 1151 cm⁻¹; ¹H NMR (CD₃OD, 400 MHz) δ 7.94-8.00 (m, 2H), 7.56 (s, 1H), 7.30-7.35 (m, 2H), 3.78 (s, 2H), 2.93 (t, J=7.0 Hz, 2H), 2.36 (s 3H), 1.48 (pent, J=6.9 Hz, 2H), 1.35 (sext, J=7.0 Hz, 2H), 0.91 (t, J=7.2, 3H); ¹³C NMR (CD₃OD, 100 MHz) 167.8, 165.2 (d, J=251.4 Hz), 136.2 (d, J=3.4 Hz), 135.8, 134.6, 131.2, 129.8 (d, J=9.3 Hz), 128.6, 115.8 (d, J=23.1 Hz), 45.1, 42.6, 31.2, 19.4, 12.5, 11.1; Anal. Calcd for C₁₇H₂₂FN₃O₅S₃: C, 44.05; H, 4.78; N, 9.06. Found: C, 44.10; H, 4.70; N, 9.05.

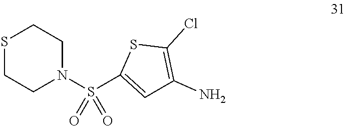 [SMILES: Nc1cc(S(=O)(=O)N2CCSCC2)sc1Cl]

2-Chloro-5-(1,4-thiazinan-4-ylsulfonyl) thien-3-ylamine (31): Thiomorpholine (0.086 mL, 0.859 mmol), and DIPEA (0.149 mL, 0.859 mmol) was dissolved in dry DCM (11.8 mL, 0.08M) and cooled to 0° C. 2-Chloro-3-nitrothiophene-5-sulfonyl chloride (0.250 g, 0.954 mmol) was added dropwise to and the mixture was heated to rt and stirred for 1 hr until TLC indicated reaction was completed. The mixture was diluted in DCM, washed with sat. NaHCO₃, followed by brine and the organics were dried over Na₂SO₄, filtered and concentrated. The crude mixture was purified via silica gel chromatography using a 20% EA/hexanes eluent to yield 4-[(5-chloro-4-nitrothiophen-2-yl)sulfonyl] thiomorpholine (0.157 g, 50%). mp=139-143° C.; TLC R_{f}=0.50 (20% EA/hexanes); IR (ATR) 3106, 2972, 2906, 1531, 1358, 1152, 566, 552 cm⁻¹; ¹H NMR (acetone-d₆, 400 MHz) δ 8.06 (s, 1H), 3.50 (t, J=4.0 Hz, 4H), 2.79 (t, J=5.1 Hz, 4H); ¹³C NMR (DMSO-d₆, 100 MHz) 143.9, 137.4, 133.2, 128.2, 48.2, 26.8; Anal. Calcd for C₈H₉ClN₂O₄S₃: C, 29.22; H, 2.76; N, 8.52. Found: C, 29.42; H, 2.40; N, 8.35. 4-[(5-chloro-4-nitrothiophen-2-yl)sulfonyl] thiomorpholine (0.67 g, 2.04 mmol) was dissolved in acetic acid (6.8 mL, 0.3M) and iron powder (0.57 g, 10.19 mmol) was added. The mixture was heated to 60° C. and stirred for 1 h, after which the acetic acid was removed in vacuo. The residue was dissolved in EA and washed with saturated aq. NaHCO₃. The organic layer was then washed with brine, dried over MgSO₄, filtered and concentrated. The residue was purified via silica gel chromatography using a 50% EA/hexanes eluent yielding 31 (0.420 g, 69%) as a yellow powder. (31) mp=138-140° C.; TLC R_{f}=0.58 (50% EA/hexanes); IR (ATR) 3448, 3360, 2903, 2852, 1611, 1569, 1405, 1334, 1149 cm⁻¹; ¹H NMR (acetone-d₆, 400 MHz) δ 7.16 (s, 1H), 5.00 (bs, 2H), 3.36 (t, J=4.9 Hz, 4H), 2.77 (t, J=5.3 Hz, 4H); ¹³C NMR (DMSO-d₆, 100 MHz) 143.9, 131.6, 124.5, 106.7, 48.1, 26.8; Anal. Calcd for C₈H₁₁ClN₂O₂S₃: C, 32.16; H, 3.71; N, 9.37. Found: C, 32.31; H, 3.40; N, 9.11.

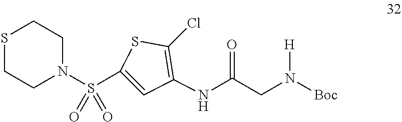 [SMILES: CC(C)(C)OC(=O)NCC(=O)Nc1cc(S(=O)(=O)N2CCSCC2)sc1Cl]

tert-Butyl-N-({[2-chloro-5-(thiomorpholine-4-sulfonyl)thiophen-3-yl]carbamoyl}methyl) carbamate (32). Boc-Glycine (0.300 g, 1.69 mmol), aminothiophene 31 (0.420 g, 1.41 mmol), HATU (1.069 g, 2.81 mmol) and DIPEA (0.49 mL, 2.81) were dissolved in 7 mL of dry DMA under argon. The mixture was stirred for 24 h at rt. The reaction was then diluted with EA, washed with sat aq. NH₄Cl (3×) and 5% aq. LiCl (3×15 mL). The organic layer was dried over MgSO₄, filtered and concentrated. The residue was purified via silica gel chromatography using 50% EA/hexanes yielding 32 a yellow solid (0.400 g, 63%). mp=143-148° C.; TLC R_{f}=0.36 (50% EA/hexanes); IR (ATR) 3297, 2982, 2915, 1693, 1147 cm⁻¹; ¹H NMR (DMSO-d₆, 400 MHz) δ 10.01 (s, 1H), 7.93 (s, 1H), 7.12 (t, J=6.1 Hz, 1H), 3.80 (d, J=5.7 Hz, 2H), 3.29 (t, J=4.4 Hz, 4H), 2.72 (t, J=5.0, 4H), 1.40 (s, 9H); ¹³C NMR (DMSO-d₆, 100 MHz) δ 169.2, 156.4, 134.8, 131.9, 128.4, 120.8, 78.7, 48.3, 43.7, 28.7, 26.8. Anal. Calcd for C₁₅H₂₂ClN₃O₅S₃: C, 39.51; H, 4.86; N, 9.22. Found: C, 39.73; H, 4.62; N, 9.07.

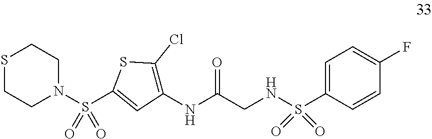 [SMILES: O=C(CNS(=O)(=O)c1ccc(F)cc1)Nc1cc(S(=O)(=O)N2CCSCC2)sc1Cl]

N-[2-Chloro-5-(thiomorpholine-4-sulfonyl)thiophen-3-yl]-2-(4-fluorobenzenesulfonamido) acetamide (33). Carbamate 32 (0.250 g, 0.532 mmol) was dissolved in 2 mL of TFA and stirred at rt for 0.5 h. The solvent was removed in vacuo and the resulting off-white solid was dissolved in dry dichloromethane (0.829 mL, 0.6M). Dry TEA (0.163 mL, 1.170 mmol) was added followed by 4-fluorobenzenesulfonyl chloride (0.114 g, 0.585 mmol). After 24 h at rt, the reaction mixture was diluted with EA, washed with water (3×5 mL) and 5% aq. HCl solution. The organic layer was dried over MgSO₄, filtered and concentrated. The residue was purified by precipitation from dichloromethane yielding 33 as a white powder (0.032 g, 12%). mp=173-178° C.; TLC R_{f}=0.24 (50% EA/hexanes); IR (ATR) 3274, 2917, 1664, 1576, 1392, 1332, 1141 cm⁻¹; ¹H NMR (CD₃OD, 400 MHz) δ 7.93-7.96 (m, 2H), 7.80 (s, 1H), 7.27-7.32 (m, 2H), 3.84 (s, 2H), 3.36 (t, J=4.9, 4H), 2.73 (t, J=4.9 Hz, 4H); ¹³C NMR (CD₃OD, 100 MHz) δ0.167.4, 165.2 (d, J=252.9 Hz), 136.3 (d, J=3.1 Hz), 133.3, 132.7, 129.9 (d, J=10.7 Hz), 127.3, 121.7, 115.8 (d, J=21.4 Hz), 45.0, 29.3, 26.7; Anal. Calcd for C₁₆H₁₇FN₃O₅S₄: C, 37.39; H, 3.33; N, 8.18. Found: C, 37.51; H, 3.02; N, 8.12.

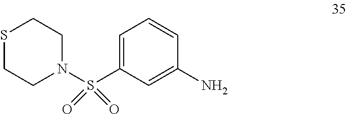 [SMILES: Nc1cccc(S(=O)(=O)N2CCSCC2)c1]

3-(1,4-Thiazinan-4-ylsulfonyl) aniline (35): Thiomorpholine (2.00 mL, 19.85 mmol) and 3-nitrobenzenesulfonyl chloride (2.00 g, 9.02 mmol) were dissolved in 8.75 mL of 1,4-dioxane and heated to 60° C. The mixture was stirred for 1 h at 60° C., after which the reaction was cooled to rt and 20 ml of water was added. The mixture was extracted with DCM (3×20 mL), and the organics were dried over MgSO4, filtered and concentrated. The residue was purified via silica gel chromatography with 20% EA/hexanes yielding 4-(3-nitrophenylsulfonyl)-1,4-thiazinane as an off-white solid (1.21 g, 47%). mp=151-155° C.; TLC R_{f}=0.33 (20% EA/hexanes); IR (ATR) 3104, 3073, 2918, 2860, 1531, 1356, 1341 cm⁻¹; ¹H NMR (CDCl₃, 400 MHz) δ 8.58 (t, J=1.8 Hz, 1H), 8.47 (ddd, J=8.3, 2.0, 0.8 Hz, 1H), 8.07 (dt, J=7.8, 0.8 Hz, 1H), 7.78 (t, J=8.1 Hz, 1H), 3.42 (t, J=4.5 Hz, 4H), 2.74 (t, J=4.8, 4H); ¹³C NMR (CDCl₃, 100 MHz) δ 148.5, 139.5, 132.8, 130.7, 127.4, 122.4, 47.9, 27.3. Anal. Calcd for C₁₀H₁₂N₂O₄S₂: C, 41.66; H, 4.20; N, 9.72. Found: C, 41.68; H, 4.46; N, 9.54. The 4-(3-nitrophenylsulfonyl)-1,4-thiazinane (1.20 g, 4.16 mmol) was dissolved in acetic acid (13.9 mL, 0.3M) and iron powder (1.16 g, 20.81 mmol) was added. The mixture was heated to 60° C. and stirred for 1 h, after which the acetic acid was removed in vacuo. The residue was dissolved in EA and washed with saturated NaHCO₃ until a pH of 8 was reached. The organics were washed with brine, dried over MgSO₄, filtered and concentrated. This gave 3-(1,4-thiazinan-4-ylsulfonyl) aniline (35) as an off-white solid (0.750 g, 69%). (35) mp=150-152° C.; TLC R_{f}=0.43 (20% EA/hexanes); IR (ATR) 3467, 3375, 2907, 2859, 2360, 1619, 1597, 1313, 1151, 716, 579 cm⁻¹; ¹H NMR (DMSO-d₆, 400 MHz) δ 7.25 (t, J=7.9 Hz, 1H), 6.91 (t, J=1.9 Hz, 1H), 6.82-6.84 (m, 1H), 6.78-6.81 (m, 1H), 5.65 (bs, 2H), 3.17 (t, J=4.9 Hz, 4H), 2.67 (t, J=5.1, 4H); ¹³C NMR (DMSO-d₆, 100 MHz) δ 150.1, 136.9, 130.3, 118.4, 114.2, 111.8, 48.3, 26.9. Anal. Calcd for C₁₀H₁₄N₂O₂S₂: C, 46.49; H, 5.46; N, 10.84. Found: C, 46.63; H, 5.17; N, 10.96.

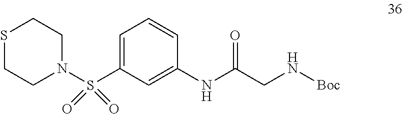 [SMILES: CC(C)(C)OC(=O)NCC(=O)Nc1cccc(S(=O)(=O)N2CCSCC2)c1]

tert-Butyl-N-([{3-(thiomorpholine-4-sulfonyl)phenyl] carbamoyl}methyl) carbamate (36). Boc-Glycine (0.244 g, 1.39 mmol), aniline 35 (0.300 g, 1.16 mmol), HATU (0.883 g, 2.32 mmol) and DIPEA (0.59 mL, 2.32) were dissolved in 7 mL of dry DMA under argon. The mixture was stirred for 24 h at rt. The reaction was diluted with EA, washed with washed with sat aq. NH₄Cl solution (3×) followed by washing with 5% aq. LiCl (3×15 mL). The organic layer was dried over MgSO₄, filtered and concentrated. The residue was purified via silica gel chromatography using 50% EA/hexanes yielding 36 a white solid (0.230 g, 48%). mp=169-173° C.; TLC R_{f}=0.44 (50% EA/hexanes); IR (ATR) 3657, 3436, 3267, 2980, 1702, 1673, 1499, 1163 cm⁻¹; ¹H NMR (CD₃OD, 400 MHz) δ 8.14 (bs, 1H), 7.77 (d, J=8.0 Hz, 1H), 7.55 (t, J=8.0 Hz, 1H), 7.48 (d, J=7.9 Hz, 1H), 3.88 (s, 2H), 3.32-3.35 (m, 4H), 2.68 (t, J=5.1 Hz, 4H), 1.47 (s, 9H); ¹³C NMR (CD₃OD, 100 MHz) δ 169.4, 156.4, 140.3, 137.1, 130.6, 123.7, 122.1, 117.6, 78.6, 48.3, 44.3, 28.7, 26.8. Anal. Calcd for C₁₇H₂₅N₃O₅S₂: C, 49.14; H, 6.06; N, 10.11. Found: C, 49.06; H, 6.35; N, 9.76.

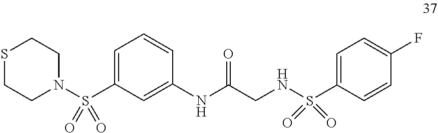 [SMILES: O=C(CNS(=O)(=O)c1ccc(F)cc1)Nc1cccc(S(=O)(=O)N2CCSCC2)c1]

2-(4-Fluorobenzenesulfonamido)-N-[3-(thiomorpholine-4-sulfonyl)phenyl]acetamide (37). Carbamate 36 (0.20 g, 0.466 mmol) was dissolved in 2 mL of TFA and stirred at rt for 0.5 h. The solvent was then removed in vacuo and the resulting off-white solid was dissolved in dry dichloromethane (0.700 mL, 0.6 M). Dry TEA (0.143 mL, 1.025 mmol) and 4-fluorobenzenesulfonyl chloride (0.100 g, 0.513 mmol) were added and the mixture was stirred at rt for 24 h. The reaction mixture was then diluted with EA, washed with water (3×5 mL) and 5% aq. HCl. The organic layer was dried over MgSO₄, filtered and concentrated. The residue was purified by precipitation from dichloromethane yielding 37 as a white powder (0.059 g, 27%). mp=143-148° C.; TLC R_{f}=0.44 (50% EA/hexanes); IR (ATR) 3318, 3284, 3221, 3137, 2918, 1697, 1590, 1548, 1314, 1148 cm⁻¹; ¹H NMR (acetone-d₆, 400 MHz) δ 9.57 (s, 1H), 8.11 (s, 1H), 7.97-8.01 (m, 2H), 7.83 (dd, J=8.2, 0.9 Hz, 1H), 7.57 (t, J=7.8 Hz, 1H), 7.47 (d, J=7.8 Hz, 1H), 7.33-7.37 (m, 2H), 6.99 (bs, 1H), 3.86 (d, J=5.9 Hz, 2H), 3.30 (t, J=4.9 Hz, 4H), 2.70 (t, J=4.0, 4H); ¹³C NMR (acetone-d₆, 100 MHz) δ 166.7, 165.0 (d, J=250.0 Hz), 139.4, 137.7, 136.6 (d, J=3.1 Hz), 130.1 (d, J=10.7 Hz), 129.8, 123.4, 122.4, 118.0, 116.1 (d, J=23.1 Hz), 48.1, 46.3, 26.8. Anal. Calcd for C₁₈H₂₀FN₃O₅S₃: C, 45.65; H, 4.26; N, 8.87. Found: C, 45.43; H, 4.30; N, 8.80.

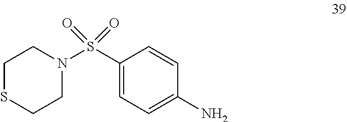 [SMILES: Nc1ccc(S(=O)(=O)N2CCSCC2)cc1]

4-(1,4-Thiazinan-4-ylsulfonyl) aniline (39): Thiomorpholine (2.00 mL, 19.85 mmol) and 4-nitrobenzenesulfonyl chloride (2.00 g, 9.02 mmol) was dissolved in 8.75 mL of 1,4-dioxane and heated to 60° C. The mixture was stirred for 1 h at 60° C., after which the reaction was cooled to rt and 20 ml of water was added. The mixture was extracted with DCM (3×20 mL), and the organics were dried over MgSO₄, filtered and concentrated. The crude mixture was purified via precipitation from DCM yielded 4-(4-nitrophenylsulfonyl)-1,4-thiazinane a white solid (1.72 g, 66%). mp=146-150° C.; TLC R_{f}=0.41 (20% EA/hexanes); IR (ATR) 3103, 2924, 2871, 1527, 1348, 1160, 1090, 748, 596 cm⁻¹; ¹H NMR (CDCl₃, 400 MHz) δ 8.39 (d, J=8.6 Hz, 2H), 7.93 (d, J=8.8 Hz, 2H), 3.31 (t, J=4.6 Hz, 4H), 2.72 (t, J=4.6, 4H); ¹³C NMR (CDCl₃, 100 MHz) δ 150.2, 143.1, 128.5, 124.5, 47.9, 27.3. Anal. Calcd for C₁₀H₁₂N₂O₄S₂: C, 41.66; H, 4.20; N, 9.72. Found: C, 41.92; H, 4.01; N, 9.86. The 4-(4-nitrophenylsulfonyl)-1,4-thiazinane (1.70 g, 5.90 mmol) was dissolved in acetic acid (19.7 mL, 0.3M) and iron powder (1.65 g, 29.48 mmol) was added. The mixture was heated to 60° C. and stirred for 1 h, after which the acetic acid was removed via vacuo. The residue was dissolved in EA and washed with saturated NaHCO₃ until a pH of 8 was reached. The organics were washed with brine, dried over MgSO₄, filtered and concentrated. This gave aniline 39 as an off-white solid was used without further purification (1.17 g, 77%). (39) mp=176-179° C.; TLC R_{f}=0.53 (20% EA/hexanes); IR (ATR) 3445, 3358, 3344, 2844, 1633, 1594, 1313, 1148, 896 cm⁻¹; ¹H NMR (acetone-d₆, 400 MHz) δ 7.74 (d, J=8.8 Hz, 2H), 6.79 (d, J=8.8 Hz, 2H), 5.55 (bs, 2H), 3.21 (t, J=5.0 Hz, 4H), 2.68 (t, J=5.0, 4H); ¹³C NMR (acetone-d₆, 100 MHz) δ 152.9, 129.5, 122.8, 113.2, 48.1, 26.9. Anal. Calcd for C₁₀H₁₄N₂O₂S₂: C, 46.49; H, 5.46; N, 10.84. Found: C, 46.73; H, 5.64; N, 10.68.

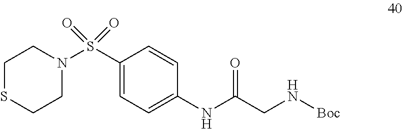 [SMILES: CC(C)(C)OC(=O)NCC(=O)Nc1ccc(S(=O)(=O)N2CCSCC2)cc1]

tert-Butyl-N-([{4-(thiomorpholine-4-sulfonyl)phenyl] carbamoyl}methyl) carbamate (40): Boc-Glycine (0.408 g, 2.32 mmol), aniline 39 (0.500 g, 1.94 mmol), HATU (1.472 g, 3.87 mmol) and DIPEA (0.99 mL, 3.87) were dissolved in 9.7 mL of dry DMA under argon. The mixture was stirred for 24 h at rt. The reaction mixture was then diluted with EA, washed with sat aq. NH₄Cl solution (3×) and 5% aq. LiCl (3×15 mL). The organic layer was then dried over MgSO₄, filtered and concentrated. The residue was purified via silica gel chromatography using 50% EA/hexanes, yielding carbamate 40 as a white powder (0.170 g, 21%). mp=180-182° C.; TLC R_{f}=0.31 (50% EA/hexanes); IR (ATR) 3369, 3280, 3195, 3114, 2977, 2853, 1675, 1495, 1158 cm⁻¹; ¹H NMR (CD₃OD, 400 MHz) δ 7.84 (d, J=8.5 Hz, 2H), 7.73 (d, J=8.5 Hz, 2H), 3.91 (s, 2H), 3.29-3.33 (m, 4H), 2.70 (t, J=5.0, 4H), 1.49 (s, 9H); ¹³C NMR (DMSO-d₆, 100 MHz) δ 169.6, 156.4, 143.6, 130.1, 129.0, 119.4, 78.6, 48.2, 44.4, 28.7, 26.8. Anal. Calcd for C₁₇H₂₅N₃O₅S₂: C, 49.14; H, 6.06; N, 10.11. Found: C, 49.38; H, 6.32; N, 10.48.

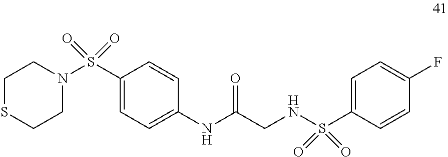 [SMILES: O=C(CNS(=O)(=O)c1ccc(F)cc1)Nc1ccc(S(=O)(=O)N2CCSCC2)cc1]

2-(4-Fluorobenzenesulfonamido)-N-[4-(thiomorpholine-4-sulfonyl)phenyl] acetamide (41). Carbamate 40 (0.170 g, 0.396 mmol) was dissolved in 2 mL of TFA and stirred at rt for 0.5 h. The solvent was then removed in vacuo and the resulting off-white solid was dissolved in dry dichloromethane (0.595 mL, 0.6M). Dry TEA (0.121 mL, 0.871 mmol) and 4-fluorobenzenesulfonyl chloride (0.085 g, 0.436 mmol) were added and the mixture was stirred at rt for 24 h. The reaction was then diluted with EA, washed with water (3×5 mL) and 5% aq. HCl. The organic layer was dried over MgSO₄, filtered and concentrated. The residue was purified by precipitation from dichloromethane yielding 41 as an off-white solid (0.068 g, 36%). mp=168-173° C.; TLC R_{f}=0.39 (50% EA/hexanes); IR (ATR) 3650, 3326, 3288, 3209, 2980, 2888, 1700, 1589, 1540, 1147, 1090 cm⁻¹; ¹H NMR (acetone-d₆, 400 MHz) δ 9.61 (s, 1H), 7.97-8.00 (m, 2H), 7.83 (d, J=8.6 Hz, 2H), 7.70 (d, J=8.6 Hz, 2H), 7.32-7.37 (m, 2H), 6.99 (bs, 1H), 3.88 (d, J=5.6 Hz, 2H), 3.28 (t, J=4.7 Hz, 4H), 2.69 (t, J=5.0, 4H); 13C NMR (acetone-d₆, 100 MHz) δ 166.8, 165.0 (d, J=254.8 Hz), 142.6, 136.7 (d, J=3.4 Hz), 131.5, 130.1 (d, J=10.0 Hz), 128.6, 119.2, 116.1 (d, J=23.1 Hz), 48.1, 46.3, 26.8. Anal. Calcd for C₁₈H₂₀FN₃O₅S₃: C, 45.65; H, 4.26; N, 8.87. Found: C, 45.34; H, 4.23; N, 8.66.

### General Procedure for the Synthesis of Sulfonamides 48-53:

The Boc protected amine was dissolved in TFA and stirred at rt for 0.5 h. The solvent was then removed in vacuo to provide the amine TFA salt. This amine salt was suspended in dry DCM (0.3 M) and dry TEA was added (2.2 equiv). 4-Fluorobenzenesulfonyl chloride (1.1 equiv) was then added and the mixture was stirred at rt for 24 h under argon. The reaction was then taken up in EA, washed with H₂O and 5% HCl. The organic layer was then dried over Na₂SO₄, filtered, and concentrated. The residue was purified by trituration from DCM or by silica gel chromatography yielding the pure product.

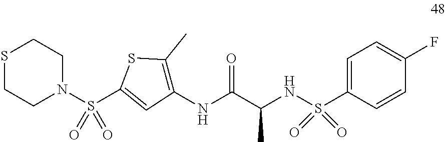 [SMILES: Cc1sc(S(=O)(=O)N2CCSCC2)cc1NC(=O)[C@H](C)NS(=O)(=O)c1ccc(F)cc1]

(2S)-2-(4-Fluorobenzenesulfonamido)-N-[2-methyl-5-(thiomorpholine-4-sulfonyl)thiophen-3-yl]propanamide (48). White powder (0.118 g, 52%); mp=178-181° C.; TLC R_{f}=0.35 (50% EA/hexanes); IR (ATR) 3296, 3247, 3102, 3066, 2912, 1654, 1336 cm⁻¹; ¹H NMR (400 MHz, CD₃OD) d 7.93-7.98 (m, 2H), 7.43 (s, 1H), 7.30 (t, J=8.7 Hz, 2H), 4.03 (q, J=7.0 Hz, 1H), 3.35-3.31 (m, 4H), 2.74 (t, J=5.4 Hz, 4H), 2.36 (s, 3H), 1.35 (d, J=7.0 Hz, 3H); ¹³C NMR (CD₃OD, 100 MHz) δ 170.4, 164.5 (d, J=249.4 Hz), 137.7 (d, J=3.0 Hz), 134.2, 133.0, 130.2, 130.0 (d, J=12.8 Hz), 129.6, 116.5 (d, J=22.6 Hz), 52.2, 48.2, 26.8, 19.6, 12.8; Anal calcd for C₁₈H₂₂FN₃O₅S₄: C, 42.59; H, 4.37; N, 8.28. Found: C, 42.55; H, 4.08; N, 8.20.

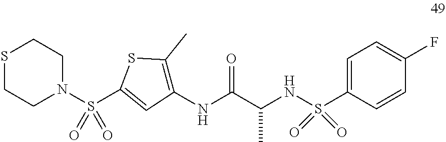 [SMILES: Cc1sc(S(=O)(=O)N2CCSCC2)cc1NC(=O)[C@@H](C)NS(=O)(=O)c1ccc(F)cc1]

(2R)-2-(4-Fluorobenzenesulfonamido)-N-[2-methyl-5-(thiomorpholine-4-sulfonyl)thiophen-3-yl]propanamide (49). White foam (0.124 g, 57%); mp=178-181° C.; TLC Rf=0.35 (50% EA/hexanes); IR (ATR) 3297, 3247, 3101, 3066, 2958, 2952, 2912, 1654, 1289 cm⁻¹; ¹H NMR (400 MHz, CD₃OD) d 7.93-7.98 (m, 2H), 7.43 (s, 1H), 7.29 (t, J=8.6 Hz, 2H), 4.04 (q, J=7.2 Hz), 3.34 (t, J=5.0 Hz, 4H), 2.74 (t, J=5.3 Hz, 4H), 2.36 (s, 3H), 1.35 (d, J=7.1 Hz, 3H); ¹³C NMR (100 MHz, CD₃OD) d 170.4, 164.5 (d, J=249.4 Hz), 137.7 (d, J=3.0 Hz), 134.2, 133.0, 130.2, 130.0 (d, J=12.8 Hz), 129.6, 116.5 (d, J=22.6 Hz), 52.2, 48.2, 26.8, 19.6, 12.8; Anal Calcd for C₁₈H₂₂FN₃O₅S₄, C, 42.59, H, 3.37, N, 8.28. Found: C, 42.88, H, 4.14, N, 8.38.

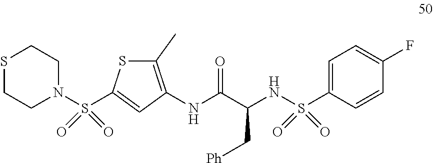 [SMILES: Cc1sc(S(=O)(=O)N2CCSCC2)cc1NC(=O)[C@H](Cc1ccccc1)NS(=O)(=O)c1ccc(F)cc1]

(2S)-2-(4-Fluorobenzenesulfonamido)-N-[2-methyl-5-(thiomorpholine-4-sulfonyl)thiophen-3-yl]-3-phenylpropanamide (50). White powder (62 mg, 23%); mp=197-202° C.; TLC R_{f}=0.38 (40% EtOAc/hexanes); IR (ATR) 3246, 2922, 2854, 2360, 1652, 1336, 1157, 1139 cm⁻¹; ¹H NMR (400 MHz, acetone-d6) δ 9.02 (bs, 1H), 7.82-7.81 (m, 2H), 7.55 (s, 1H), 7.23-7.18 (m, 7H), 7.14 (bs, 1H), 4.31 (t, J=6.7 Hz, 1H), 3.33-3.30 (m, 4H), 3.13-3.08 (m, 1H), 2.99-2.94 (m, 1H), 2.77-2.75 (m, 4H), 2.23 (s, 3H); ¹³C NMR (100 MHz, acetone-d6) δ 169.6, 165.6 (d, J=253.1 Hz), 138.0 (d, J=3.9 Hz), 137.4, 134.3, 133.2, 131.8, 130.8 (d, J=9.2 Hz), 130.3, 129.8, 129.2, 127.6, 116.8 (d, J=23.2 Hz), 59.2, 49.0, 39.9, 27.7, 12.4; Anal. Calcd for C₂₄H₂₆FN₃O₅S₄: C, 49.38; H, 4.49; N, 7.20. Found: C, 49.47; H, 4.31; N, 7.03.

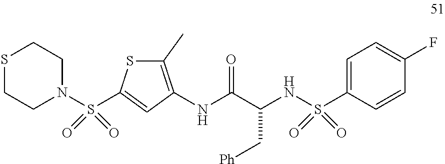 [SMILES: Cc1sc(S(=O)(=O)N2CCSCC2)cc1NC(=O)[C@@H](Cc1ccccc1)NS(=O)(=O)c1ccc(F)cc1]

(2R)-2-(4-Fluorobenzenesulfonamido)-N-[2-methyl-5-(thiomorpholine-4-sulfonyl)thiophen-3-yl]-3-phenylpropanamide (51). White powder (86 mg, 23%); mp=199-204° C.; TLC R_{f}=0.33 (40% EA/hexanes); IR (ATR) 3262, 2913, 2850, 1676, 1333, 1150 cm⁻¹; ¹H NMR (400 MHz, CD₃CN) δ 8.14 (s, 1H), 7.77-7.73 (m, 2H), 7.36 (s, 1H), 7.27-7.12 (m, 7H), 6.29 (d, J=9.2 Hz, 1H), 4.19-4.13 (m, 1H), 3.29-3.26 (m, 4H), 3.06-3.01 (m, 1H), 2.92-2.86 (m, 1H), 2.73-2.70 (m, 4H), 2.17 (s, 3H); ¹³C NMR (100 MHz, acetone-d6) δ 169.6, 165.7 (d, J=252.2 Hz), 138.1 (d, J=3.3 Hz), 137.4, 134.3, 133.3, 131.8, 130.8 (d, J=9.4 Hz), 130.3, 129.8, 129.2, 127.6, 116.8 (d, J=22.9 Hz), 59.3, 49.0, 40.0, 27.7, 12.4; Anal. Calcd for C₂₄H₂₆FN₃O₅S₄: C, 49.38; H, 4.49; N, 7.20. Found: C, 49.47; H, 4.35; N, 7.23.

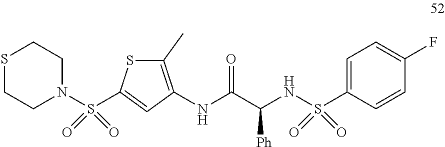 [SMILES: Cc1sc(S(=O)(=O)N2CCSCC2)cc1NC(=O)[C@@H](NS(=O)(=O)c1ccc(F)cc1)c1ccccc1]

(2S)-2-(4-Fluorobenzenesulfonamido)-N-[2-methyl-5-(thiomorpholine-4-sulfonyl)thiophen-3-yl]-2-phenylacetamide (52). White foam (0.135 g, 71%); mp=80-84° C.; TLC R_{f}=0.53 (50% EA/hexanes); IR (ATR) 3265, 2980, 2888, 2359, 2341. 1676, 1336 cm⁻¹; ¹H NMR (400 MHz, DMSO-d₆) δ 10.08 (s, 1H), 8.87 (d, J=9.8 Hz, 1H), 7.81 (q, J=7.8 Hz, 2H), 7.38-7.42 (m, 3H), 7.23-7.33 (m, 5H), 5.29 (d, J=9.7 Hz, 1H), 3.19 (t, J=4.5 Hz, 4H), 2.70 (t, J=4.9 Hz, 4H), 2.24 (s, 3H); ¹³C NMR (DMSO-d₆, 100 MHz) δ 168.0, 164.5 (d, J=249.2 Hz), 137.6 (d, J=2.9 Hz), 137.2, 134.3, 132.7, 130.2, 130.2, 130.1, 128.9, 128.8 (d, J=83.8 Hz), 127.5, 116.3 (d, J=22.5 Hz), 55.4, 48.2, 26.8, 12.8; Anal calcd for C₂₃H₂₄FN₃O₅S₄: C, 48.49; H, 4.25; N, 7.38. Found: C, 48.12; H, 4.06; N, 7.74.

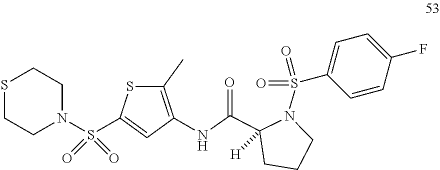 [SMILES: Cc1sc(S(=O)(=O)N2CCSCC2)cc1NC(=O)[C@@H]1CCCN1S(=O)(=O)c1ccc(F)cc1]

(2S)-1-(4-Fluorobenzenesulfonyl)-N-[2-methyl-5-thiomorpholine-4-sulfonyl)thiophen-3-yl]pyrrolidine-2-carboxamide (53). White power (0.160 g, 60%); mp=119-123° C.; TLC R_{f}=0.34 (50% EA/hexanes); IR (ATR) 3342, 2980, 2359, 2341, 1684, 1338 cm⁻¹; ¹H NMR (400 MHz, DMSO-d₆) δ 9.81 (s, 1H), 7.96 (q, J=5.55 Hz, 2H), 7.63 (s, 1H), 7.50 (t, J=8.8 Hz, 2H), 4.25 (t, J=6.7 Hz, 1H), 3.46-3.53 (m, 1H), 3.24 (t, J=4.9 Hz, 4H), 2.72 (t, J=4.9 Hz, 4H), 2.40 (s, 3H), 1.87-1.96 (m, 3H), 1.56-1.65 (m, 1H), 1.24 (s, 1H); ¹³C NMR (100 MHz, DMSO-d₆) δ 170.5, 165.2 (d, J=250.4 Hz), 135.3, 133.9, 133.3, 130.8, 130.5 (d, J=90.9 Hz), 130.2, 117.1 (d, J=22.5 Hz), 61.7, 49.6, 48.3, 31.5, 26.8, 24.7, 13.0; Anal calcd for C₂₀H₂₄FN₃O₅S₄: C, 45.01; H, 4.53; N, 7.87. Found: C, 45.38; H, 4.73; N, 7.53.

### General Procedure for the Synthesis of 64-73:

The amine salt 21 was suspended in dry DCM (0.7 M) and dry TEA (2.2 equiv) was added. The electrophile (sulfonyl chloride, benzyl chloride, or chloroformate, (1.1 equiv)) was added and the mixture was stirred at rt for 24 h under argon. The reaction then taken up in EA, washed with H₂O and 5% aq. HCl. The organic layer was dried over Na₂SO₄, filtered, and concentrated. The residue was then purified by trituration from DCM or by silica gel chromatography yielding the pure product.

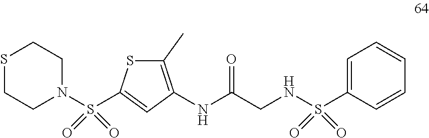 [SMILES: Cc1sc(S(=O)(=O)N2CCSCC2)cc1NC(=O)CNS(=O)(=O)c1ccccc1]

2-Benzenesulfonamido-N-[2-methyl-5-(thiomorpholine-4-sulfonyl)thiophen-3-yl]acetamide (64). White powder (0.074 g, 27%); mp=172-176° C.; TLC R_{f}=0.26 (50% EA/hexanes); IR (ATR) 3352, 3160, 2980, 2889, 1682, 1587, 1329, 1156 cm⁻¹; ¹H NMR (DMSO-d₆, 400 MHz) δ 9.72 (s, 1H), 8.12 (t, J=6.4 Hz, 1H), 7.82 (d, J=7.5 Hz, 2H), 7.55-7.65 (m, 4H), 3.70 (d, J=6.2 Hz, 2H), 3.20 (t, J=4.8 Hz, 4H), 2.71 (t, J=4.5 Hz, 4H), 2.32 (s, 3H); ¹³C NMR (DMSO-d₆, 100 MHz) 166.9, 140.8, 134.0, 133.2, 133.0, 130.0, 129.6, 129.6, 127.1, 48.3, 45.7, 26.8, 12.9; Anal. Calcd for C₁₇H₂₁N₃O₅S₄: C, 42.93; H, 4.45; N, 8.84. Found: C, 42.65; H, 4.06; N, 8.50.

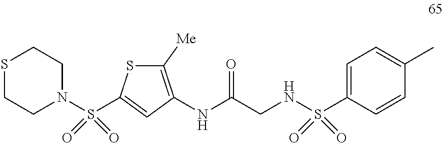 [SMILES: Cc1ccc(S(=O)(=O)NCC(=O)Nc2cc(S(=O)(=O)N3CCSCC3)sc2C)cc1]

N-[2-methyl-5-(thiomorpholine-4-sulfonyl)thiophen-3-yl]-2-{[(4-methylbenzenesulfonyl) carbamoyl]amino}acetamide (65). White powder (0.037 g, 12%); mp=192-195° C.; TLC R_{f}=0.39 (10% MeOH/DCM); IR (ATR) 3657, 3329, 3107, 2980, 2889, 2473, 1708, 1655, 1333, 1148 cm⁻¹; ¹H NMR (acetone-d₆, 400 MHz) δ 9.08 (s, 1H), 7.92 (d, J=8.3 Hz, 2H), 7.83 (s, 1H), 7.41 (d, J=8.4 Hz, 2H), 6.97 (bs, 1H), 4.04 (d, J=5.0 Hz, 4H), 3.33 (t, J=5.0 Hz, 4H), 2.75 (t, J=5.0 Hz, 4H), 2.43 (s, 3H), 2.37 (s, 3H); ¹³C NMR (acetone-d₆, 100 MHz) 167.1, 151.4, 144.3, 137.7, 132.9, 132.8, 130.9, 129.5, 129.0, 127.5, 48.2, 43.2, 26.8, 20.6, 11.6; Anal. Calcd for C₁₉H₂₄N₄O₆S₄: C, 48.60; H, 4.94; N, 8.95. Found: C, 48.85; H, 4.64; N, 9.07.

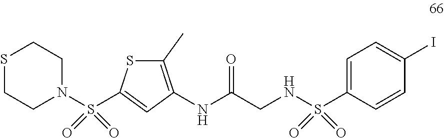 [SMILES: Cc1sc(S(=O)(=O)N2CCSCC2)cc1NC(=O)CNS(=O)(=O)c1ccc(I)cc1]

2-(4-Iodobenzenesulfonamido)-N-[2-methyl-5-(thiomorpholine-4-sulfonyl)thiophen-3-yl]acetamide (66). White powder (0.094 g, 27%); mp=198-202° C.; TLC R_{f}=0.51 (50% EA/hexane); IR (ATR) 3381, 3156, 2979, 2907, 2865, 1655, 1335, 1327, 1164, 1148 cm⁻¹; ¹H NMR (DMSO-d₆, 400 MHz) δ 9.73 (s, 1H), 8.20 (t, J=5.9 Hz, 1H), 7.97 (d, J=8.1 Hz, 2H), 7.59 (d, J=5.0 Hz, 2H), 7.57 (s, 1H), 3.71 (d, J=6.2 Hz, 2H), 3.22 (t, J=4.6 Hz, 4H), 2.71 (t, J=4.6 Hz, 4H), 2.32 (s, 3H); ¹³C NMR (DMSO-d₆, 100 MHz) 166.8, 140.5, 138.5, 134.1, 133.2, 130.1, 129.6, 128.8, 101.0, 48.3, 45.6, 26.8, 12.9; Anal. Calcd for C₁₇H₂₀IN₃O₅S₄: C, 33.95; H, 3.35; N, 6.99. Found: C, 33.93; H, 3.42; N, 6.65.

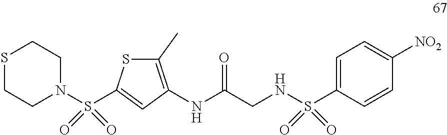 [SMILES: Cc1sc(S(=O)(=O)N2CCSCC2)cc1NC(=O)CNS(=O)(=O)c1ccc([N+](=O)[O-])cc1]

N-[2-methyl-5-(thiomorpholine-4-sulfonyl)thiophen-3-yl]-2-(4-nitrobenzenesulfonamido) acetamide (67). White powder (0.059 g, 20%); mp=209-212° C.; TLC R_{f}=0.189 (50% EA/hexanes); IR (ATR) 3380, 3247, 3105, 3063, 2980, 2914, 2853, 1668, 1531, 1349, 1332, 1150 cm⁻¹; ¹H NMR (acetone-d₆, 400 MHz) δ 9.73 (s, 1H), 8.21 (t, J=5.6 Hz, 1H), 7.98 (d, J=7.5 Hz, 2H), 7.60 (d, J=5.7 Hz, 2H), 7.57 (s, 1H), 3.72 (d, J=5.3 Hz, 2H), 3.22 (t, J=4.1 Hz, 4H), 2.71 (t, J=4.7 Hz, 4H), 2.32 (s, 3H); ¹³C NMR (acetone-d₆, 100 MHz) 166.8, 140.5, 138.5, 134.1, 133.2, 130.1, 129.5, 128.8, 101.0, 48.3, 45.6, 26.8, 12.9; Anal. Calcd for C₁₇H₂₀N₄O₇S₄: C, 39.22; H, 3.87; N, 10.76. Found: C, 39.14; H, 3.84; N, 11.14.

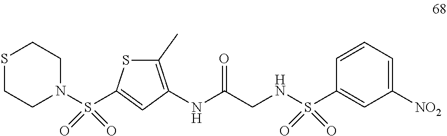 [SMILES: Cc1sc(S(=O)(=O)N2CCSCC2)cc1NC(=O)CNS(=O)(=O)c1cccc([N+](=O)[O-])c1]

N-[2-methyl-5-(thiomorpholine-4-sulfonyl)thiophen-3-yl]-2-(3-nitrobenzenesulfonamido) acetamide (68). White powder (0.16 g, 53%); mp=141-144° C.; TLC R_{f}=0.19 (50% EA/hexanes); IR (ATR) 3365, 3217, 3104, 2927, 2851, 1677, 1528, 1426, 1349, 1337, 1161 cm⁻¹; ¹H NMR (acetone-d₆, 400 MHz) δ 9.13 (s, 1H), 8.68 (t, J=1.8 Hz, 1H), 8.50 (dd, J=1.3, 8.3 Hz, 1H), 8.32 (d, J=7.8 Hz, 1H), 7.93 (t, J=7.9 Hz, 1H), 7.63 (s, 1H), 7.37 (bs, 1H), 4.00 (d, J=5.5 Hz, 2H), 3.29 (t, J=4.9 Hz, 4H), 2.75 (t, J=4.9 Hz, 4H), 2.37 (s, 3H); ¹³C NMR (acetone-d₆, 100 MHz) 165.8, 148.3, 142.4, 133.1, 133.0, 132.6, 131.0, 130.9, 128.8, 127.1, 122.1, 48.1, 45.6, 26.8, 11.6; Anal. Calcd for C₁₇H₂₀N₄O₇S₄: C, 39.22; H, 3.87; N, 10.76. Found: C, 39.23; H, 3.91; N, 10.94.

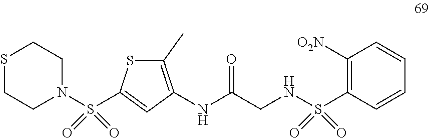 [SMILES: Cc1sc(S(=O)(=O)N2CCSCC2)cc1NC(=O)CNS(=O)(=O)c1ccccc1[N+](=O)[O-]]

N-[2-Methyl-5-(thiomorpholine-4-sulfonyl)thiophen-3-yl]-2-(2-nitrobenzenesulfonamido) acetamide (69). Orange powder (0.140 g, 47%); mp=101-104° C.; TLC R_{f}=0.19 (50% EA/hexanes); IR (ATR) 3325, 3097, 2917, 2490, 1686, 1537, 1336, 1149 cm⁻¹; ¹H NMR (CD₃OD, 400 MHz) δ 8.11-8.15 (m, 1H), 7.90-7.93 (m, 1H), 7.79 (s, 1H), 4.03 (s, 2H), 3.31-3.33 (m, 4H), 2.72 (t, J=5.2 Hz, 4H), 2.36 (s, 3H); ¹³C NMR (CD₃OD, 100 MHz) 167.8, 148.0, 135.4, 133.8, 133.4, 132.4, 131.9, 131.0, 130.3, 129.3, 124.8, 48.0, 45.4, 26.7, 11.2; Anal. Calcd for C₁₇H₂₀N₄O₇S₄: C, 39.22; H, 3.87; N, 10.76. Found: C, 39.60; H, 4.11; N, 10.48.

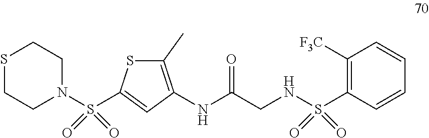 [SMILES: Cc1sc(S(=O)(=O)N2CCSCC2)cc1NC(=O)CNS(=O)(=O)c1ccccc1C(F)(F)F]

N-[2-methyl-5-(thiomorpholine-4-sulfonyl)thiophen-3-yl]-2-(2-trifluoromethylbenzene sulfonamido) acetamide (70). Off-white foam (0.030 g, 24%); mp=89-93° C.; TLC R_{f}=0.32 (50% EA/50% hexanes); IR (ATR) 3329, 2980, 1685, 1581, 1307, 1143, 1116 cm⁻¹; ¹H NMR (acetone-d₆, 400 MHz) δ 9.07 (s, 1H), 8.27 (dd, J=3.7, 5.0 Hz, 1H), 8.00 (dd, J=3.7, 5.5 Hz, 1H), 7.86 (dd, J=6.0, 3.7 Hz, 1H), 7.65 (bs, 1H), 3.99 (d, J=6.0 Hz, 2H), 3.30 (t, J=4.6 Hz, 4H), 2.74 (t, J=5.0 Hz, 4H), 2.37 (s, 3H); ¹³C NMR (acetone-d₆, 100 MHz) 166.0, 138.9 (q, J=1.1 Hz), 133.2, 133.1, 132.8, 131.2, 130.0, 128.9, 128.5 (q, J=6.3 Hz), 127.3, 127.2 (q, J=32.8 Hz), 123.3 (q, J=271.6 Hz), 48.1, 45.7, 26.8, 11.7; Anal. Calcd for C₁₈H₂₀F₃N₃O₅S₄: C, 39.77; H, 3.71; N, 7.73. Found: C, 39.74; H, 3.95; N, 7.42.

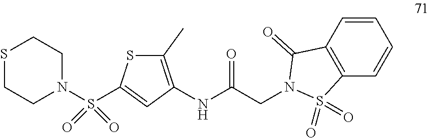 [SMILES: Cc1sc(S(=O)(=O)N2CCSCC2)cc1NC(=O)CN1C(=O)c2ccccc2S1(=O)=O]

N-[2-methyl-5-(thiomorpholine-4-sulfonyl)thiophen-3-yl]-2-(1,1,3-trioxo-2,3-dihydro-1-benzothiazol-2-yl) acetamide (71). White powder (0.041 g, 36%); mp=234-237° C.; TLC R_{f}=0.28 (50% EA/hexanes); IR (ATR) 3264, 2980, 2918, 1743, 1671, 1335, 1150, 585 cm⁻¹; ¹H NMR (acetone-d₆, 400 MHz) δ 9.32 (s, 1H), 8.22 (d, J=7.7 Hz, 1H), 8.05-8.17 (m, 3H), 7.80 (s, 1H), 4.64 (s, 2H), 3.31 (bs, 4H), 2.74 (t, J=5.0 Hz, 4H), 2.44 (s, 3H); ¹³C NMR (acetone-d₆, 100 MHz) 163.4, 158.9, 137.9, 135.7, 135.0, 133.8, 132.6, 131.1, 129.0, 127.2, 125.1, 121.3, 48.2, 40.4, 26.8, 11.7; Anal. Calcd for C₁₈H₁₉N₃O₆S₄: C, 43.10; H, 3.82; N, 8.38. Found: C, 43.35; H, 3.98; N, 8.17.

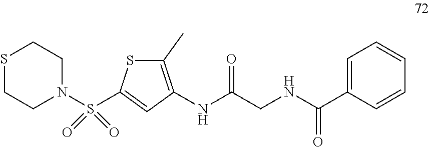 [SMILES: Cc1sc(S(=O)(=O)N2CCSCC2)cc1NC(=O)CNC(=O)c1ccccc1]

N-[2-methyl-5-(thiomorpholine-4-sulfonyl)thiophen-3-yl]-2-(phenylformamido) acetamide (72). White powder (0.079 g, 31%); mp=189-193° C.; TLC R_{f}=0.53 (5% MeOH/DCM); IR (ATR) 3309, 3098, 3066, 2907, 2860, 2467, 1675, 1636, 1556, 1137, 1072, 909 cm⁻¹; ¹H NMR (acetone-d₆, 400 MHz) δ 9.70 (s, 1H), 8.65 (s, 1H), 8.41 (d, J=7.5 Hz, 2H), 8.30 (s, 1H), 8.01 (t, J=7.5 Hz, 1H), 7.94 (t, J=7.1 Hz, 2H), 4.68 (d, J=5.2 Hz, 2H), 3.77 (t, J=4.9 Hz, 4H), 3.19 (t, J=4.9 Hz, 4H), 2.87 (s, 3H); ¹³C NMR (acetone-d₆, 100 MHz) 167.5, 167.3, 134.2, 133.2, 132.7, 131.5, 130.9, 129.0, 128.4, 127.3, 48.2, 43.6, 26.8, 11.7; Anal. Calcd for C₁₈H₂₁N₃O₄S₃: C, 49.18; H, 4.82; N, 9.56. Found: C, 49.00; H, 5.02; N, 9.22.

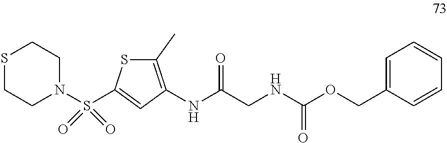 [SMILES: Cc1sc(S(=O)(=O)N2CCSCC2)cc1NC(=O)CNC(=O)OCc1ccccc1]

Benzyl N-({[2-methyl-5-(thiomorpholine-4-sulfonyl)thiophen-3-yl]carbamoyl}methyl) carbamate (73). White powder (0.079 g, 31%); mp=69-74° C.; TLC R_{f}=0.21% (50% EA/hexanes); IR (ATR) 3309, 3098, 3066, 2962, 2907, 2860, 2466, 1676, 1636, 1556, 1350, 1333, 1137 cm⁻¹; ¹H NMR (DMSO-d₆, 400 MHz) δ 9.78 (s, 1H), 7.74 (s, 1H), 7.58 (t, J=5.9 Hz, 1H), 7.31-7.38 (m, 5H), 5.06 (s, 2H), 3.85 (d, J=5.8 Hz, 2H), 3.23 (t, J=4.6 Hz, 4H), 2.71 (t, J=4.6 Hz, 4H), 2.39 (s, 3H); ¹³C NMR (DMSO-d₆, 100 MHz) 168.5, 157.1, 137.5, 133.7, 133.5, 129.9, 129.7, 128.8, 128.3, 128.2, 66.0, 48.3, 44.0, 26.8, 13.0; Anal. Calcd for C₁₈H₂₁N₃O₄S₃: C, 49.18; H, 4.82; N, 9.56. Found: C, 49.00; H, 5.02; N, 9.22.

TABLE 5
Malachite Data on Bis-sulfonamide Hybrids:

| Com- | Notebook | | Malachite |
|---|---|---|---|
| pound | Ref | Structure | (SHIP1) |
| K420 | REA-5-002 | 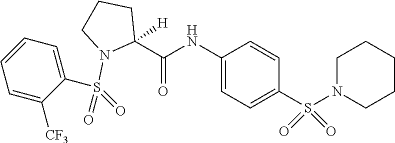 [SMILES: O=C(Nc1ccc(S(=O)(=O)N2CCCCC2)cc1)[C@@H]1CCCN1S(=O)(=O)c1ccccc1C(F)(F)F] | 138% (500 μM) 112% (250 μM) 99% (125 μM) |
| K421 | REA-5-014 | 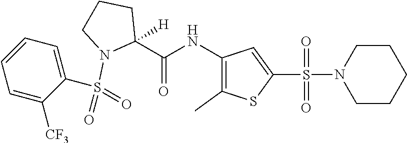 [SMILES: Cc1sc(S(=O)(=O)N2CCCCC2)cc1NC(=O)[C@@H]1CCCN1S(=O)(=O)c1ccccc1C(F)(F)F] | 158% (500 μM) 122% (250 μM) 96% (125 μM) |
| K422 | REA-5-031 | 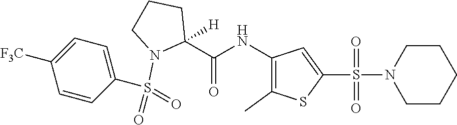 [SMILES: Cc1sc(S(=O)(=O)N2CCCCC2)cc1NC(=O)[C@@H]1CCCN1S(=O)(=O)c1ccc(C(F)(F)F)cc1] | 126% (500 μM) 103% (250 μM) 96% (125 μM) |
| K423 | REA-5-032 | 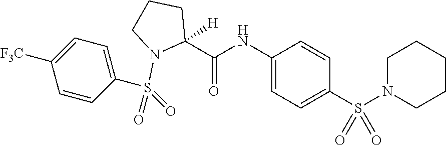 [SMILES: O=C(Nc1ccc(S(=O)(=O)N2CCCCC2)cc1)[C@@H]1CCCN1S(=O)(=O)c1ccc(C(F)(F)F)cc1] | 136% (500 μM) 109% (250 μM) 99% (125 μM) |
| K424 | REA-5-033 | 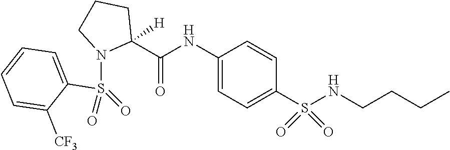 [SMILES: CCCCNS(=O)(=O)c1ccc(NC(=O)[C@@H]2CCCN2S(=O)(=O)c2ccccc2C(F)(F)F)cc1] | 91% (500 μM) 84% (250 μM) 79% (125 μM) |
| K425 | REA-5-034 | 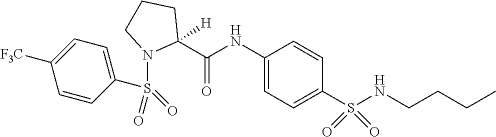 [SMILES: CCCCNS(=O)(=O)c1ccc(NC(=O)[C@@H]2CCCN2S(=O)(=O)c2ccc(C(F)(F)F)cc2)cc1] | 81% (500 μM) 80% (250 μM) 75% (125 μM) |
| K426 | REA-5-065 | 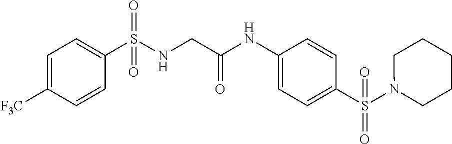 [SMILES: O=C(CNS(=O)(=O)c1ccc(C(F)(F)F)cc1)Nc1ccc(S(=O)(=O)N2CCCCC2)cc1] | 74% (500 μM) 63% (250 μM) 75% (125 μM) |
| K427 | REA-5-066 | 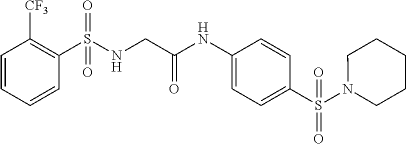 [SMILES: O=C(CNS(=O)(=O)c1ccccc1C(F)(F)F)Nc1ccc(S(=O)(=O)N2CCCCC2)cc1] | 116% (500 μM) 88% (250 μM) 80% (125 μM) |

### Hybrid Compound Data:

### RA-5-002 K420:

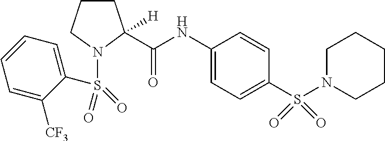 [SMILES: O=C(Nc1ccc(S(=O)(=O)N2CCCCC2)cc1)[C@@H]1CCCN1S(=O)(=O)c1ccccc1C(F)(F)F]

Yield: 66%. IR (film) 3333, 3110, 2939, 1700, 1591 cm-1; TLC R_{f}=0.25 (50% EA/50% hexanes); mp=98-102° C.; 1H NMR (400 MHz, CDCl3) δ 8.59 (s, 1H), 8.12 (dd, J=7.0, 2.0 Hz, 2H), 7.86 (dd, J=6.5, 2.3 Hz, 2H), 7.71-7.65 (m, 2H), 7.60 (q, J=8.7 Hz, 4H), 4.42 (dd, J=7.8, 2.2 Hz, 1H), 3.55-3.47 (m, 1H), 3.44-3.36 (m, 1H), 2.89 (t, J=5.2 Hz, 4H), 2.41-2.33 (m, 1H), 2.03-1.85 (m, 3H), 1.56 (p, J=5.47 Hz, 4H), 1.38-1.30 (m, 2H); 13C {1H} NMR (100 MHz, CDCl3) δ 169.2, 141.0, 136.4, 133.6, 132.7, 132.1, 131.5, 128.8, 128.4, 128.1, 123.8, 119.3, 62.7, 49.7, 46.9, 30.2, 25.1, 24.6, 23; Anal calcd for C₂₃H₂₆F₃N₃O₅S₂: C, 50.63; H, 4.80; N, 7.70. Found: C, 50.99; H, 4.64; N, 7.98.

### RA-5-014 K421

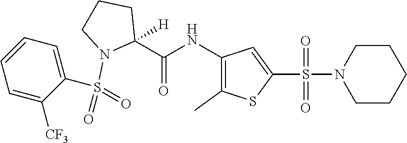 [SMILES: Cc1sc(S(=O)(=O)N2CCCCC2)cc1NC(=O)[C@@H]1CCCN1S(=O)(=O)c1ccccc1C(F)(F)F]

Yield: 72%. IR (film) 3338, 2926, 1685, 1579 cm-1; TLC Rf=0.22 (50% EA/50% hexanes); mp=103-106° C.; 1H NMR (400 MHz, CDCl3) 08.41 (bs, 1H), 8.16 (t, J=4.5 Hz, 1H), 7.95 (t, J=4.6 Hz, 1H), 7.79-7.75 (m, 3H), 4.49-4.45 (m, 1H), 3.63-3.57 (m, 1H), 3.42-3.33 (m, 1H), 3.03 (t, J=5.5 Hz, 4H), 2.47-2.41 (m, 1H), 2.38 (s, 3H), 2.03-1.90 (m, 3H), 1.66 (p, J=5.5 Hz, 4H), 1.45 (p, J=5.88 Hz, 2H); ¹³C{1H} NMR (100 MHz, CDCl3) δ 168.4, 136.3, 133.5, 132.7, 132.6, 131.9, 131.6, 130.9, 128.8 (q, J=6.6 Hz, 1C), 128.1, 122.3 (q, J=274 Hz, 1C), 62.5, 49.5, 47.0, 30.0, 25.0, 24.6, 23.3, 12.3; Anal calcd for C₂₃H₂₆F₃N₃O₅S₃: C, 46.72; H, 4.63; N, 7.43. Found: C, 46.62; H, 4.54; N, 7.33.

### RA-5-031 K422

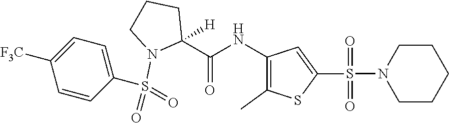 [SMILES: Cc1sc(S(=O)(=O)N2CCCCC2)cc1NC(=O)[C@@H]1CCCN1S(=O)(=O)c1ccc(C(F)(F)F)cc1]

Yield: 57%. IR (film) 3342, 3102, 2942, 1685, 1578 cm-1; TLC Rf=0 . . . 46 (50% EA/50% hexanes); mp=110-114° C.; 1H NMR (400 MHz, CDCl3) δ 8.63 (s, 1H), 8.01 (d, J=8.2 Hz, 2H), 7.87 (d, J=8.4 Hz, 2H), 7.84 (s, 1H), 4.18 (dd, J=8.7, 2.4 Hz, 1H), 3.72-3.65 (m, 1H), 3.24-3.16 (m, 1H), 3.05 (t, J=5.4 Hz, 4H), 4.25 (s, 3H), 2.42-2.35 (m, 1H), 1.96-1.82 (m, 1H), 1.78-1.57 (m, 6H), 1.49-1.41 (m, 2H); 13C{1H} NMR (100 MHz, CDCl3) δ 168.1, 138.8, 132.7, 131.7, 131.1, 128.4, 128.2, 126.7, 124.3, 121.6, 62.6, 50.2, 47.0, 29.6, 25.0, 24.5, 23.4, 12.4; Anal calcd for C₂₃H₂₆F₃N₃O₅S₃: C, 46.72; H, 4.63; N, 7.43. Found: C, 46.83; H, 4.73; N, 7.29.

### RA-5-032 K423

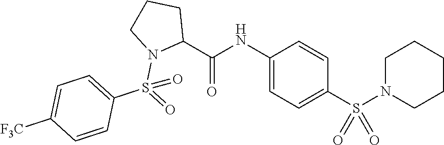 [SMILES: O=C(Nc1ccc(S(=O)(=O)N2CCCCC2)cc1)C1CCCN1S(=O)(=O)c1ccc(C(F)(F)F)cc1]

Yield: 50%. IR (film) 3332, 3105, 2942, 1708, 1592 cm-1; TLC Rf=0 . . . 46 (50% EA/50% hexanes); mp=190-194° C.; 1H NMR (400 MHz, CDCl3) δ 8.92 (s, 1H), 8.02 (d, J=8.3 Hz, 2H), 7.87 (d, J=8.3 Hz, 2H), 7.75 (dd, J=14.2, 9.0 Hz, 4H), 4.19 (dd, J=8.6, 2.7 Hz, 1H), 3.73-3.66 (m, 1H), 3.28-3.20 (m, 1H), 2.97 (t, J=5.3 Hz, 4H), 2.43-2.35 (m, 1H), 1.94-1.83 (m, 1H), 1.79-1.70 (m, 1H), 1.63 (p, J=5.5 Hz, 5H), 1.41 (p, J=5.7 Hz, 2H); 13C{1H} NMR (100 MHz, CDCl₃) δ 168.9, 141.1, 138.9, 135.4 (q, J=33.4 Hz, 1C), 131.6, 128.9, 128.4, 126.7 (q, J=3.6 Hz, 1C), 122.95, (q, J=273 Hz, 1C), 119.5, 63.0, 50.2, 46.9, 29.6, 25.1, 24.5, 23.5; Anal calcd for C₂₃H₂₆F₃N₃O₅S₂: C, 50.63; H, 4.80; N, 7.70. Found: C, 50.31; H, 4.89; N, 8.08.

### RA-5-033 K424

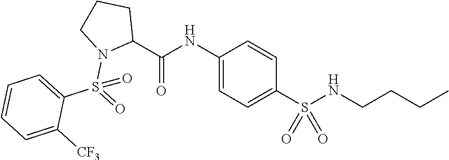 [SMILES: CCCCNS(=O)(=O)c1ccc(NC(=O)C2CCCN2S(=O)(=O)c2ccccc2C(F)(F)F)cc1]

K424 Is an oil, no melt temp. Yield: 72%. IR (film) 3292, 3113, 2959, 1691, 1592, cm-1; TLC Rf=0 . . . 41 (60% EA/40% hexanes); 1H NMR (400 MHz, CDCl3) 08.66 (bs, 1H), 8.22-8.18 (m, 1H), 7.95-7.90 (m, 1H), 7.78 (d, J=8.6 Hz, 2H), 7.76-7.71 (m, 2H), 7.62 (d, J=8.7 Hz, 2H), 4.50 (dd, J=5.5, 3.0 Hz, 1H), 4.39 (bs, 1H), 3.62-3.54 (m, 1H), 3.52-3.44 (m, 1H), 2.92 (t, J=7.0 Hz, 2H), 2.47-2.39 (m, 1H), 2.11-2.00 (m, 1H), 2.00-1.92 (m, 2H), 1.77-1.67 (m, 1H), 1.44 (p, J=7.4 Hz, 2H), 1.34-1.21 (m, 2H), 8.85 (t, J=7.2 Hz, 3H); 13C{1H} NMR (100 MHz, CDCl3) 0169.2, 141.0, 136.4, 135.2, 133.6, 132.7, 132.1, 128.84 (q, J=6.7 Hz, 1C), 128.2 (t, J=15.8 Hz, 1C), 122.4 (q, J=274 Hz, 1C), 119.5, 62.7, 49.7, 42.9, 31.5, 30.3, 24.7, 19.7, 13.5; Anal calcd for C₂₃H₂₆F₃N₃O₅S₂: C, 49.52; H, 4.91; N, 7.88. Found: C, 49.30; H, 4.60; N, 7.60.

### RA-5-034 K425

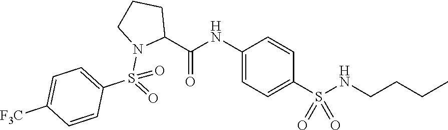 [SMILES: CCCCNS(=O)(=O)c1ccc(NC(=O)C2CCCN2S(=O)(=O)c2ccc(C(F)(F)F)cc2)cc1]

Yield: 62%. IR (film) 3351, 3106, 2959, 1683, 1586 cm-1; TLC Rf=0 . . . 41 (60% EA/40% hexanes); mp=157-160° C.; 1H NMR (400 MHz, CDCl3) 08.94 (s, 1H), 8.03 (d, J=8.25 Hz, 2H), 7.87 (d, J=8.51 Hz, 2H), 7.82 (d, J=8.51 Hz, 2H), 7.72 (d, J=8.8 Hz, 2H), 4.56 (bs, 1H), 4.21 (dd, J=8.5, 2.4 Hz, 1H) 3.73-3.68 (m, 1H), 3.24, (q, J=9.4 Hz, 1H), 2.93 (q, J=6.4 Hz, 2H), 2.40-2.35 (m, 1H), 1.95-1.83 (m, 1H), 1.79-1.61 (m, 1H), 1.45, (p, J=7.4 Hz, 2H), 1.34-1.23 (m, 3H), 0.86 (t, J=7.3 Hz, 3H); 13C{1H} NMR (100 MHz, CDCl3) δ 169.0, 141.1, 138.9, 135.4 (q, J=33.4 Hz, 1C), 128.4, 128.3, 126.7 (q, J=3.7 Hz, 1C), 122.9 (q, J=273 Hz, 1C), 119.8, 63.0, 50.2, 42.9, M 31.5, 29.7, 24.5, 19.7, 13.5; Anal calcd for C₂₃H₂₆F₃N₃O₅S₂: C, 49.52; H, 4.91; N, 7.88. Found: C, 49.58; H, 5.03; N, 8.04.

### RA-5-065 K426

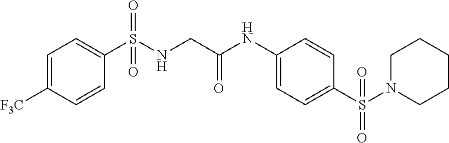 [SMILES: O=C(CNS(=O)(=O)c1ccc(C(F)(F)F)cc1)Nc1ccc(S(=O)(=O)N2CCCCC2)cc1]

Yield: 54%. IR (film) 3376, 3263, 2940, 1677, 1594, 1440 cm-1; TLC Rf=0.82 (80% EA/20% hexanes); mp=210-214° C.; 1H NMR (400 MHz, Acetone-D6) 09.60 (bs, 1H), 8.13 (d, J=8.3 Hz, 2H), 7.94 (d, J=8.3 Hz, 2H), 7.78 (d, J=8.7 Hz, 2H), 7.67 (d, J=8.7 Hz, 2H), 3.95 (s, 2H), 2.92 (t, J=5.4 Hz, 4H), 2.77 (bs, 1H), 1.59 (p, J=5.4 Hz, 4H), 1.41 (p, J=5.9 Hz, 2H); 13C{1H} NMR (100 MHz, Acetone-D6) 0167.2 (d, J=7.8 Hz), 145.1, 143.1, 143.0, 131.8, 129.5, 128.7, 126.9 (q, J=3.8 Hz, 1C) 124.4, (q, J=271 Hz 1C), 119.7 (d, J=8.7 Hz, 1C), 47.5, 46.9, 25.8, 23.9; Anal calcd for C20H22F3N3O5S2: C, 47.58; H, 4.39; N, 8.31. Found: C, 47.78; H, 4.53; N, 8.65.

### RA-5-066 K427

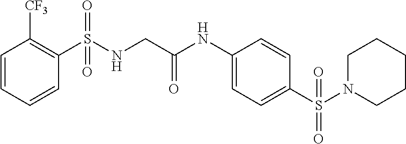 [SMILES: O=C(CNS(=O)(=O)c1ccccc1C(F)(F)F)Nc1ccc(S(=O)(=O)N2CCCCC2)cc1]

Yield: 56%. IR (film) 3329, 2979, 2845, 1715, 1528, 1495 cm-1; TLC Rf=0.76 (80% EA/20% hexanes); mp=230-235° C.; 1H NMR (400 MHz, Acetone-D6) 09.59 (bs, 1H), 8.26 (dd, J=6.0, 4.0 Hz, 1H), 8.00 (dd, J=5.0, 3.1 Hz, 1H), 7.84 (dd, J=5.9, 3.6 Hz, 2H), 7.76 (d, J=8.7 Hz, 2H), 7.67 (d, J=8.7 Hz, 2H), 4.01 (s, 2H), 2.92 (t, J=5.4 Hz, 4H), 2.78 (bs, 1H), 1.59 (p, J=5.4 Hz, 4H), 1.40 (p, J=5.9 Hz, 2H); 13C{1H} NMR (100 MHz, Acetone-D6) 0166.7, 142.3, 142.2, 139.0, 133.0, 132.7, 131.1, 131.0, 128.7, 128.4 (q, J=6.5 Hz, 1C), 123.2 (q, J=273 Hz, 1C), 118.9, 46.8, 46.2, 25.0, 23.2; Anal calcd for C20H22F3N3O5S2: C, 47.58; H, 4.39; N, 8.31. Found: C, 47.79; H, 4.21; N, 8.68.

### General Procedure for the Synthesis of Carbamates S1-S6:

Aminothiphene 3 (1 equiv), the Boc protected amino acid (1.2 equiv), HATU (2 equiv), and DIPEA (2 equiv, 0.59 mL, 2.32) were dissolved in dry DMA (0.2 M) under argon. The mixture was stirred for 24 h at rt. The reaction was diluted with EA, washed with sat aq. NH₄Cl solution (3×) followed by washing with 5% aq. LiCl (3×15 mL). The organic layer was then dried over MgSO₄, filtered and concentrated. The residue was purified via silica gel chromatography.

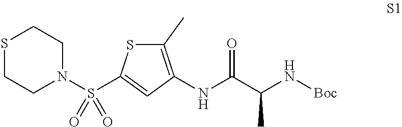 [SMILES: Cc1sc(S(=O)(=O)N2CCSCC2)cc1NC(=O)[C@H](C)NC(=O)OC(C)(C)C]

(S)-1-[N-2-Methyl-5-(1,4-thiazinan-4-ylsulfonyl) thien-3-ylcarbamoyl]ethylamino-tert-butylformylate (S1). (0.134 g, 41%). mp=62-66° C.; TLC Rf=0.51 (50% EA/hexanes); IR (film) 3305, 2976, 2918, 1671 cm⁻¹; ¹H NMR (400 MHz, CDCl₃) δ 8.66 (bs, 1H), 7.95 (s, 1H), 4.90 (d, J=6.2 Hz, 1H), 4.29 (p, J=6.7 Hz, 1H), 3.37 (t, J=4.5 Hz, 4H), 2.72 (t, J=4.8 Hz, 4H), 2.36 (s, 3H), 1.47 (s, 9H), 1.43 (d, J=7.0 Hz, 3H); ¹³C NMR (100 MHz, CD₃CN) δ 172.9, 157.1, 135.5, 134.1, 132.0, 130.5, 80.6, 51.9, 28.9, 28.1, 18.4, 13.2; Anal calcd for C₁₇H₂₇N₃O₅S₃: C, 45.42; H, 6.05; N, 9.35. Found: C, 45.37; H, 5.71; N, 9.17.

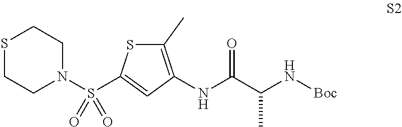 [SMILES: Cc1sc(S(=O)(=O)N2CCSCC2)cc1NC(=O)[C@@H](C)NC(=O)OC(C)(C)C]

(R)-1-[N-2-Methyl-5-(1,4-thiazinan-4-ylsulfonyl) thien-3-ylcarbamoyl]ethylamino-tert-butylformylate (S2). (0.050 g, 37%). mp=63-66° C.; TLC Rf=0.50 (50% EA/hexanes); IR (film) 3312, 2976, 2917, 1670 cm⁻¹; ¹H NMR (400 MHz, CDCl₃) δ 8.67 (bs, 1H), 7.96 (s, 1H), 4.89 (d, J=6.9 Hz, 1H), 4.28 (p, J=6.7 Hz, 1H), 3.37 (t, J=4.6 Hz, 4H), 2.72 (t, J=4.9 Hz, 4H), 2.36 (s, 3H), 1.47 (s, 9H), 1.43 (d, J=7.0 Hz, 3H); ¹³C NMR (100 MHz, CD₃CN) δ 172.9, 157.1, 135.5, 134.1, 132.0, 130.5, 80.6, 52.0, 49.5, 28.9, 28.1, 18.4, 13.2; Anal calcd for C₁₇H₂₇N₃O₅S₃: C, 45.42; H, 6.05; N, 9.35. Found: C, 45.37; H, 5.71; N, 9.17.

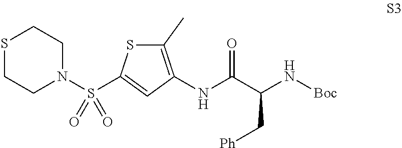 [SMILES: Cc1sc(S(=O)(=O)N2CCSCC2)cc1NC(=O)[C@H](Cc1ccccc1)NC(=O)OC(C)(C)C]

(S)-1-[N-2-methyl-5-(1,4-thiazinan-4-ylsulfonyl) thien-3-ylcarbamoyl]-2-phenylethylamino-tert-butyl formylate (44). Yellow foam (0.304 g, 64%); mp=78-83° C.; TLC R_{f}=0.66 (40% EA/hexanes); IR (ATR) 3290, 2924, 1660, 1365, 1138 cm⁻¹; ¹H NMR (400 MHz, DMSO-d₆) δ 9.76 (s, 1H), 7.60 (s, 1H), 7.30-7.21 (m, 5H), 7.15 (d, J=7.75 Hz, 1H), 4.41-4.35 (m, 1H), 3.23 (bs, 4H), 3.03-2.98 (m, 1H), 2.89-2.84 (m, 1H), 2.71 (bs, 4H), 2.30 (s, 3H), 1.34 (s, 9H); ¹³C NMR (100 MHz, CD₃CN) δ 171.3, 156.6, 138.4, 135.6, 133.5, 131.7, 130.4, 130.1, 129.4, 127.7, 80.3, 57.2, 49.1, 38.6, 28.5, 27.7, 12.9; Anal. Calcd for C₂₃H₃₁N₃O₅S₃: C, 52.55; H, 5.94; N, 7.99. Found: C, 52.53; H, 6.08; N, 8.17.

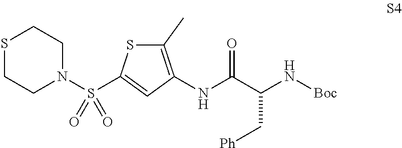 [SMILES: Cc1sc(S(=O)(=O)N2CCSCC2)cc1NC(=O)[C@@H](Cc1ccccc1)NC(=O)OC(C)(C)C]

(R)-1-[N-2-methyl-5-(1,4-thiazinan-4-ylsulfonyl) thien-3-ylcarbamoyl]-2-phenylethylamino-tert-butyl formylate (45). Yellow foam (461 mg, 80%); mp=76-80° C.; TLC R_{f}=0.88 (50% EA/hexanes); IR (ATR) 3282, 2967, 1664, 1355, 1149 cm⁻¹; ¹H NMR (400 MHz, acetone-d₆) δ 9.04 (s, 1H), 7.77 (s, 1H), 7.31-7.23 (m, 5H), 6.32 (bs, 1H), 4.54-4.49 (m, 1H), 3.33 (bs, 4H), 3.23-3.18 (m, 1H), 3.07-3.01 (m, 1H), 2.77-2.74 (m, 4H), 2.33 (s, 3H), 1.37 (s, 9H); ¹³C NMR (100 MHz, CD₃CN) δ 171.3, 156.6, 138.4, 135.6, 133.5, 131.7, 130.4, 130.1, 129.4, 127.7, 80.3, 57.2, 49.1, 38.6, 28.5, 27.7, 12.9; Anal. Calcd for C₂₃H₃₁N₃O₅S₃: C, 52.55; H, 5.94; N, 7.99. Found: C, 52.91; H, 5.69; N, 7.95.

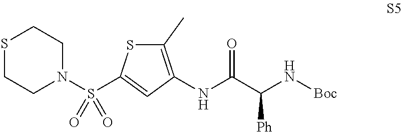 [SMILES: Cc1sc(S(=O)(=O)N2CCSCC2)cc1NC(=O)[C@@H](NC(=O)OC(C)(C)C)c1ccccc1]

{(S)—[N-2-methyl-5-(1,4-thiazinan-4-ylsulfonyl) thien-3-ylcarbamoyl]phenylmethyl}amino-tert-butyl formylate (46). (0.912 g, 61%). IR (film) 3329, 3117, 3068, 2978, 2920, 2854, 1665 cm⁻¹; TLC Rf=0.56 (50% EA/hexanes); mp=102-106° C.; ¹H NMR (400 MHz, CDCl₃) δ 7.75 (s, 1H), 7.33-7.44 (m, 5H), 3.35 (t, J=5.0 Hz, 4H), 2.71 (t, J=4.6 Hz, 4H), 2.23 (s, 3H), 1.44 (s, 9H); ¹³C NMR (100 MHz, DMSO-D6) δ 169.6, 155.5, 138.5, 134.6, 133.2, 130.1, 129.7, 128.8, 128.2, 127.8, 78.9, 58.3, 40.2, 28.6, 26.7, 12.9; Anal calcd for: C₂₂H₂₉N₃O₅S₃: C, 51.64; H, 5.71; N, 8.21. Found: C, 51.97; H, 5.50; N, 8.21.

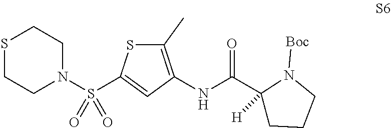 [SMILES: Cc1sc(S(=O)(=O)N2CCSCC2)cc1NC(=O)[C@@H]1CCCN1C(=O)OC(C)(C)C]

tert-Butyl(S)-2-[N-2-methyl-5-(1,4-thiazinan-4-ylsulfonyl) thien-3-ylcarbamoyl]pyrrolidine-1-carboxylate (47). (0.774 g, 90%); IR (film) 3325, 2979, 2359, 2341, 1685, 1587 cm⁻¹; TLC Rf=0.38 (50% EA/hexanes); mp=159-164° C.; 1H NMR (400 MHz, CDCl₃) δ 9.68 (bs, 1H), 8.08 (bs, 1H), 4.47 (d, J=7.1 Hz, 1H), 3.37 (t, J=4.5 Hz, 6H), 2.72 (app t, J=5.0 Hz, 4H), 2.57 (bs, 1H), 2.37 (s, 3H), 2.00-1.86 (m, 3H), 1.50 (s, 9H); ¹³C NMR (100 MHz, CDCl₃) δ 169.0, 156.9, 133.5, 130.8, 129.7, 127.8, 81.2, 59.8, 47.9, 47.3, 28.3, 26.6, 24.6, 12.2; Anal calcd for: C, 47.98; H, 6.15; N, 8.83. Found: C, 47.70; H, 5.97; N, 9.13.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising.” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

### REFERENCES

Yang, L.; Williams, D. E.; Mui, A.; Ong, C.; Krystal, G.; Van Soest, R.; Andersen, R. J. Synthesis of pelorol and analogues: activators of the inositol 5-phosphatase SHIP. Organic Letters 2005, 7, 1073-1076. Ong, C. J.; Ming-Lum, A.; Nodwell, M.; Ghanipour, A.; Yang, L.; Williams, D. E.; Kim, J.; Demirjian, L.; Qasimi, P.; Ruschmann, J.; et al. Small-molecule agonists of SHIP1 inhibit the phosphoinositide 3-kinase pathway in hematopoietic cells. Blood 2007, 110, 1942-1949. Kennah, M.; Yau, T. Y.; Nodwell, M.; Krystal, G.; Andersen, R. J.; Ong, C. J.; A, L. F. M. Activation of SHIP via a small molecule agonist kills multiple myeloma cells. Exp Hematol 2009, 37 (11), 1274-1283. DOI: 10.1016/j.exphem.2009.08.001. Williams, D. E.; Amlani, A.; Dewi, A. S.; Patrick, B. O.; van Ofwegen, L.; Mui, A. L. F.; Andersen, R. J. Australin E Isolated from the Soft Coral Cladiella sp. Collected in Pohnpei Activates the Inositol 5-Phosphatase SHIP1. Aust. J. Chem. 2010, 63 (6), 895-900. DOI: 10.1071/ch10053. Li, D.; Carr, G.; Zhang, Y.; Williams, D. E.; Amlani, A.; Bottriell, H.; Mui, A. L. F.; Andersen, R. J. Turnagainolides A and B, cyclic depsipeptides produced in culture by a Bacillus sp.: Isolation, structure elucidation, and synthesis. J. Nat. Prod. 2011, 74 (5), 1093-1099. DOI: 10.1021/np200033y. Meimetis, L. G.; Nodwell, M.; Yang, L.; Wang, X.; Wu, J.; Harwig, C.; Stenton, G. R.; Mackenzie, L. F.; MacRury, T.; Patrick, B. O.; et al. Synthesis of SHIP1-Activating Analogs of the Sponge Meroterpenoid Pelorol. European Journal of Organic Chemistry 2012, 2012 (27), 5195-5207. DOI: 10.1002/ejoc.201200631. Stenton, G. R.; Mackenzie, L. F.; Tam, P.; Cross, J. L.; Harwig, C.; Raymond, J.; Toews, J.; Chernoff, D.; MacRury, T.; Szabo, C. Characterization of AQX-1125, a small-molecule SHIP1 activator. Part 2. Efficacy studies in allergic and pulmonary inflammation models in vivo. Br. J. Pharmacol. 2013, 168 (6), 1519-1529. DOI: 10.1111/bph.12038. Stenton, G. R.; Mackenzie, L. F.; Tam, P.; Cross, J. L.; Harwig, C.; Raymond, J.; Toews, J.; Wu, J.; Ogden, N.; MacRury, T.; et al. Characterization of AQX-1125, a small-molecule SHIP1 activator. Part 1. Effects on inflammatory cell activation and chemotaxis in vitro and pharmacokinetic characterization in vivo. Br. J. Pharmacol. 2013, 168 (6), 1506-1518. DOI: 10.1111/bph. 12039. Lemm, E. A.; Valle-Argos, B.; Smith, L. D.; Richter, J.; Gebreselassie, Y.; Weston-Bell, N. J.; Karydis, L.; Stevenson, F. K.; Forconi, F.; Steele, A. J.; et al. Preclinical Evaluation of a Novel SHIP1 Phosphatase Activator for Inhibition of PI3K Signaling in Malignant B Cells. Clin. Cancer Res. 2020, 26 (7), 1700-1711. Mackenzie, L.; Macrury, T.; Harwig, C.; Khlebnikov, V.; Shan, R.; Place, S.; Bird, P.; Pettigrew, J.; Bhatti, N. A. Oxa- and thiatetracyclooctadecatriene derivatives as SHIP1 modulators and their preparation and use for the treatment of SHIP1-mediated diseases. Patent Appl. WO 2011069118, 2011. Mackenzie, L. F.; MacRury, T. B.; Harwig, C.; Bogucki, D.; Raymond, J. R.; Pettigrew, J. D. Methods related to the preparation of seco steroids as SHIP1 modulators. Application: WO 2014-US19126, 2014158654, 2014; Zhang, X.; Majerus, P. W. Phosphatidylinositol signaling reactions. Semin. Cell Dev. Biol. 1998, 9, 153-160.; Catimel, B.; Yin, M.-X.; Schieber, C.; Condron, M.; Patsiouras, H.; Catimel, J.; Robinson, D. E. J. E.; Wong, L. S.-M.; Nice, E. C.; Holmes, A. B.; Burgess, A. W. PI(3,4,5)P3 Interactome. J. Proteome Res. 2009, 8, 3712-3726; Cantley, L. C. The phosphoinositide-3-kinase pathway. Science 2002, 296, 1655-1657; Marion, F.; Williams, D. E.; Patrick, B. O.; Hollander, I.; Mallon, R.; Kim, S. C.; Roll, D. M.; Feldberg, L.; Van Soest, R.; Andersen, R. J. Liphagal, a Selective Inhibitor of PI3 Kinase a Isolated from the Sponge Aka coralliphaga: Structure Elucidation and Biomimetic Synthesis. Org. Lett. 2006, 8, 321-324.; Kerr, W. G.; Park, M. Y.; Maubert, M.; Engelman, R. W. SHIP deficiency causes Crohn's disease-like ileitis. Gut 2011, 60, 177-88. doi:10.1136/gut.2009.202283; McLarren, K. W.; Cole, A. E.; Weisser, S. B.; Voglmaier, N. S.; Conlin, V. S.; Jacobson, K.; Popescu, O.; Boucher, J. L.; Sly, L. M. SHIP-deficient mice develop spontaneous intestinal inflammation and arginase-dependent fibrosis. Am J Pathol 2011, 179, 180-8. doi:10.1016/j.ajpath.2011.03.018; Fernandes, S.; Srivastava, N.; Sudan, R.; Middleton, F. A.; Shergill, A. K.; Ryan, J. C.; Kerr, W. G. SHIP1 Deficiency in Inflammatory Bowel Disease Is Associated With Severe Crohn's Disease and Peripheral T Cell Reduction. Front Immunol 2018, 9, 1100. doi:10.3389/fimmu.2018.01100; Somasundaram, R.; Fernandes, S.; Deuring, J. J.; de Haar, C.; Kuipers, E. J.; Vogelaar, L.; Middleton, F. A.; van der Woude, C. J.; Peppelenbosch, M. P.; Kerr, W. G.; Fuhler, G. M. Analysis of SHIP1 expression and activity in Crohn's disease patients. PLOS One 2017, 12, e0182308. doi:10.1371/journal.pone.0182308; Ngoh, E. N.; Brugger, H. K.; Monajemi, M.; Menzies, S. C.; Hirschfeld, A. F.; Del Bel, K. L.; Jacobson, K.; Lavoie, P. M.; Turvey, S. E.; Sly, L. M. The Crohn's disease-associated polymorphism in ATG16L1 (rs2241880) reduces SHIP gene expression and activity in human subjects. Genes Immun 2015, 16, 452-61. doi:10.1038/gene.2015.30; Ngoh, E. N.; Weisser, S. B.; Lo, Y.; Kozicky, L. K.; Jen, R.; Brugger, H. K.; Menzies, S. C.; McLarren, K. W.; Nackiewicz, D.; van Rooijen, N.; Jacobson, K.; Ehses, J. A.; Turvey, S. E.; Sly, L. M. Activity of SHIP, Which Prevents Expression of Interleukin 1beta, Is Reduced in Patients With Crohn's Disease. Gastroenterology 2016, 150, 465-76. doi:10.1053/j.gastro.2015.09.049; Park, M. Y.; Srivastava, N.; Sudan, R.; Viernes, D. R.; Chisholm, J. D.; Engelman, R. W.; Kerr, W. G. Impaired T-cell survival promotes mucosal inflammatory disease in SHIP1-deficient mice. Mucosal Immunol 2014, 7, 1429-39. doi:10.1038/mi.2014.32; Tsai, A. P.; Lin, P. B.-C.; Dong, C.; Moutinho, M.; Casali, B. T.; Liu, Y.; Lamb, B. T.; Landreth, G. E.; Oblak, A. L.; Nho, K. INPP5D expression is associated with risk for Alzheimer's disease and induced by plaque-associated microglia. Neurobiol. Dis. 2021, 153, 105303. doi:10.1016/j.nbd.2021.105303; Viernes, D. R.; Choi, L. B.; Kerr, W. G.; Chisholm, J. D. Discovery and Development of Small Molecule SHIP Phosphatase Modulators. Med. Res. Rev. 2014, 34, 795-824. doi:10.1002/med.21305; Andersen, R. J. Sponging off nature for new drug leads. Biochem. Pharmacol. 2017, 139, 3-14. doi:10.1016/j.bcp.2017.04.01215); Chamberlain, T. C.; Cheung, S. T.; Yoon, J. S. J.; Ming-Lum, A.; Gardill, B. R.; Shakibakho, S.; Dzananovic, E.; Ban, F.; Samiea, A.; Jawanda, K.; Priatel, J.; Krystal, G.; Ong, C. J.; Cherkasov, A.; Andersen, R. J.; McKenna, S. A.; Van Petegem, F.; Mui, A. L. F. Interleukin-10 and Small Molecule SHIP1 Allosteric Regulators Trigger Anti-inflammatory Effects through SHIP1/STAT3 Complexes. iScience 2020, 23, 101433. doi:10.1016/j.isci.2020.101433; Dungan, O. M.; Dormann, S.; Fernandes, S.; Duffy, B. C.; Effiong, D. G.; Kerr, W. G.; Chisholm, J. D. Synthetic studies on the indane SHIP1 agonist AQX-1125. Org. Biomol. Chem. 2022, 20, 4016-4020. doi:10.1039/d2ob00555g; Pedicone, C.; Fernandes, S.; Matera, A.; Meyer, S. T.; Loh, S.; Ha, J.-H.; Bernard, D.; Chisholm, J. D.; Paolicelli, R. C.; Kerr, W. G. Discovery of a novel SHIP1 agonist that promotes degradation of lipid-laden phagocytic cargo by microglia. iScience 2022, 25, 104170. doi:10.1016/j.isci.2022.104170; Goodman, K. B.; Bury, M. J.; Cheung, M.; Cichy-Knight, M. A.; Dowdell, S. E.; Dunn, A. K.; Lee, D.; Lieby, J. A.; Moore, M. L.; Scherzer, D. A.; Sha, D.; Suarez, D. P.; Murphy, D. J.; Harpel, M. R.; Manas, E. S.; McNulty, D. E.; Annan, R. S.; Matico, R. E.; Schwartz, B. K.; Trill, J. J.; Sweitzer, T. D.; Wang, D.-y.; Keller, P. M.; Krawiec, J. A.; Jaye, M. C. Discovery of potent, selective sulfonylfuran urea endothelial lipase inhibitors. Bioorg. Med. Chem. Lett. 2009, 19, 27-30. doi:10.1016/j.bmcl.2008.11.033; Elliott, T. S.; Slowey, A.; Ye, Y.; Conway, S. J. The use of phosphate bioisosteres in medicinal chemistry and chemical biology. MedChemComm 2012, 3, 735-751. doi:10.1039/c2md20079a; Zhang, Y.; Borrel, A.; Ghemtio, L.; Regad, L.; Boije af Gennas, G.; Camproux, A.-C.; Yli-Kauhaluoma, J.; Xhaard, H. Structural Isosteres of Phosphate Groups in the Protein Data Bank. J. Chem. Inf. Model. 2017, 57, 499-516. doi:10.1021/acs.jcim.6b00519; Ong, C. J.; Ming-Lum, A.; Nodwell, M.; Ghanipour, A.; Yang, L.; Williams, D. E.; Kim, J.; Demirjian, L.; Qasimi, P.; Ruschmann, J.; Cao, L. P.; Ma, K.; Chung, S. W.; Duronio, V.; Anderson, R. J.; Krystal, G.; Mui, A. L. Small-molecule agonists of SHIP1 inhibit the phosphoinositide 3-kinase pathway in hematopoietic cells. Blood 2007, 110, 1942-1949.; Goto, Y.; Matsui, T.; Ozaki, S.-i.; Watanabe, Y.; Fukuzumi, S. Mechanisms of Sulfoxidation Catalyzed by High-Valent Intermediates of Heme Enzymes: Electron-Transfer vs. Oxygen-Transfer Mechanism. J. Am. Chem. Soc. 1999, 121, 9497-9502. doi:10.1021/ja9901359.; Meunier, B.; de Visser, S. P.; Shaik, S. Mechanism of Oxidation Reactions Catalyzed by Cytochrome P450 Enzymes. Chem. Rev. (Washington, DC, U. S.) 2004, 104, 3947-3980. doi:10.1021/cr020443g.; Daina, A.; Michielin, O.; Zoete, V.; Michielin, O.; Michielin, O. SwissADME: a free web tool to evaluate pharmacokinetics, drug-likeness and medicinal chemistry friendliness of small molecules. Sci Rep 2017, 7, 42717. doi:10.1038/srep42717.; Jaladanki, C. K.; Taxak, N.; Varikoti, R. A.; Bharatam, P. V. Toxicity Originating from Thiophene Containing Drugs: Exploring the Mechanism using Quantum Chemical Methods. Chem. Res. Toxicol. 2015, 28, 2364-2376. doi:10.1021/acs.chemrestox.5b00364.; Orr, S. T. M.; Ripp, S. L.; Ballard, T. E.; Henderson, J. L.; Scott, D. O.; Obach, R. S.; Sun, H.; Kalgutkar, A. S. Mechanism-Based Inactivation (MBI) of Cytochrome P450 Enzymes: Structure-Activity Relationships and Discovery Strategies To Mitigate Drug-Drug Interaction Risks. J. Med. Chem. 2012, 55, 4896-4933. doi:10.1021/jm300065h.; Gronowitz, S.; Ander, I. Base-catalyzed reaction of some methyl nitrothiophenes with aldehydes. Unexpected cyclobutane formation. Acta Chem. Scand., Ser. B 1975, B29, 513.; Nepali, K.; Lee, H.-Y.; Liou, J.-P. Nitro-Group-Containing Drugs. J. Med. Chem. 2019, 62, 2851-2893. doi:10.1021/acs.jmedchem.8b00147.; Tseng, C.-C.; Baillie, G.; Donvito, G.; Mustafa, M. A.; Juola, S. E.; Zanato, C.; Massarenti, C.; Dall'Angelo, S.; Harrison, W. T. A.; Lichtman, A. H.; Ross, R. A.; Zanda, M.; Greig, I.

What is claimed:

1. A method for treating a chronic or acute inflammatory disease comprising:
administering to a subject in need thereof, a therapeutically effective amount of one or more compounds that activate phosphatidylinositol 3,4,5-trisphosphate 5-phosphatase (SHIP), or an isoform thereof.

2. The method of claim 1, wherein treating comprises administering a therapeutically effective amount of one or more SHIP activator compounds to the subject.

3. The method of claim 1, wherein the inflammatory disease is chronic obstructive pulmonary disease (COPD), inflammatory bowel disease (IBD), rheumatoid arthritis (RA), or septic shock.

4. The method of claim 1, wherein the inflammatory disease is Alzheimer's disease.

5. The method of claim 1, wherein the SHIP activator compound is 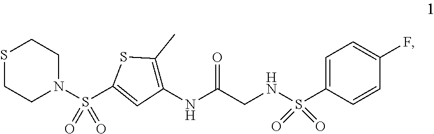 [SMILES: Cc1sc(S(=O)(=O)N2CCSCC2)cc1NC(=O)CNS(=O)(=O)c1ccc(F)cc1]
or an analog thereof.

6. The method of claim 1, wherein the one or more SHIP activator compound is selected from the group consisting of: 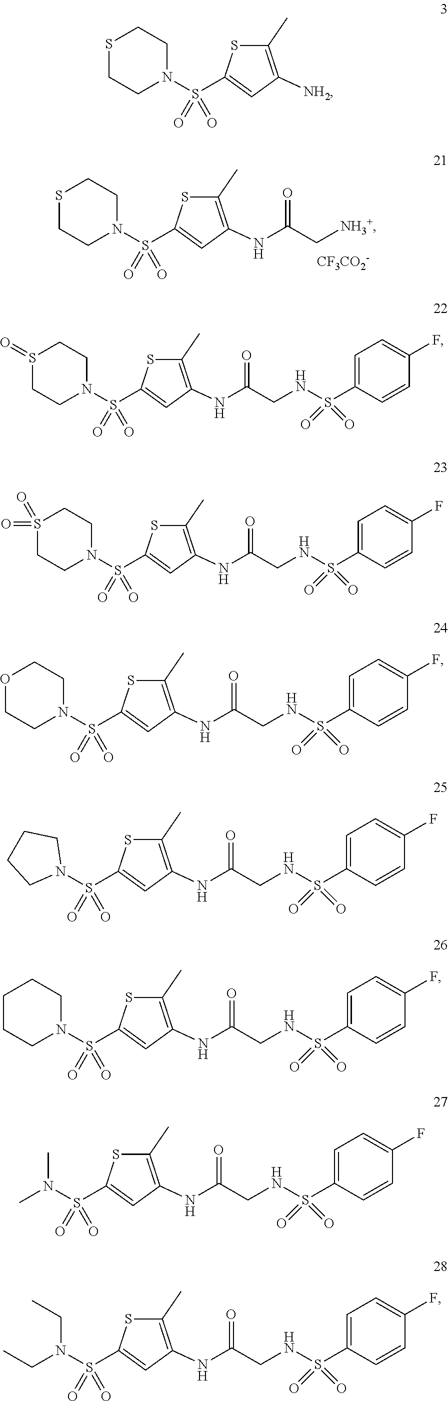 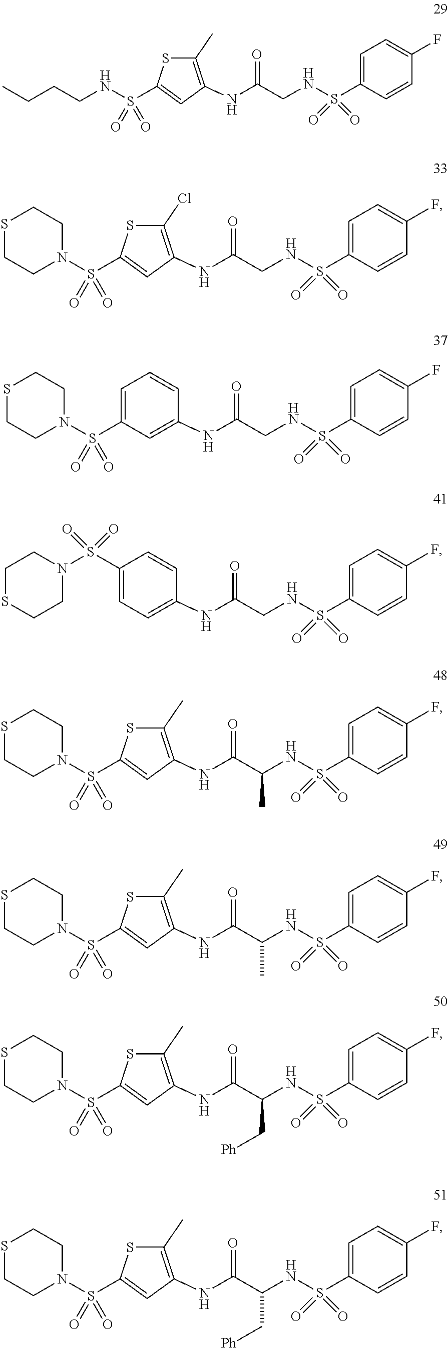 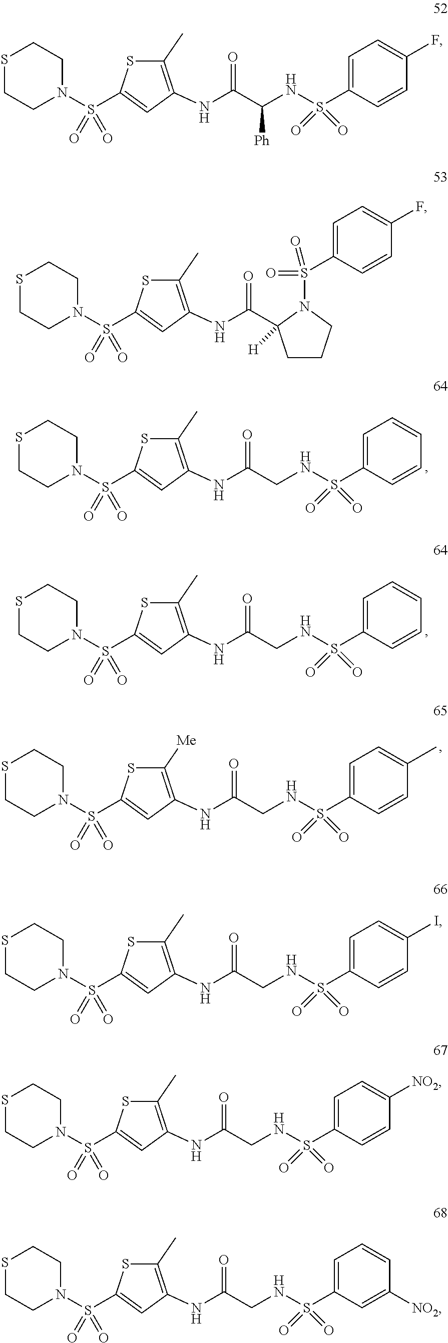 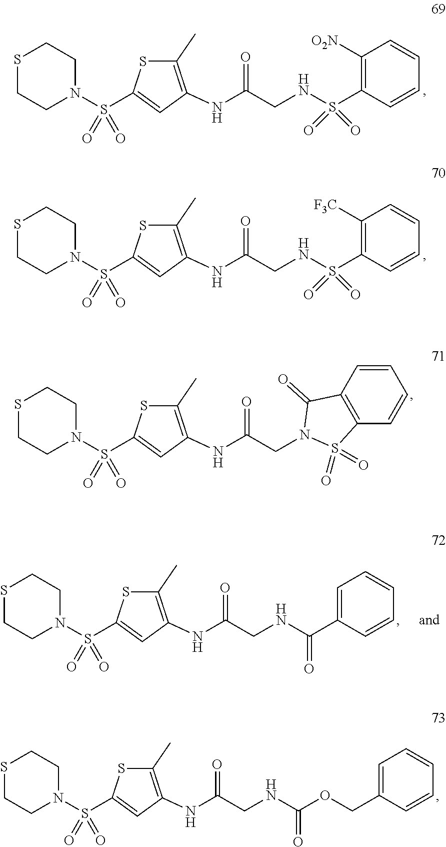 [SMILES: Cc1sc(S(=O)(=O)N2CCSCC2)cc1NC(=O)CNS(=O)(=O)c1ccccc1[N+](=O)[O-] ; Cc1sc(S(=O)(=O)N2CCSCC2)cc1NC(=O)CNS(=O)(=O)c1ccccc1C(F)(F)F ; Cc1sc(S(=O)(=O)N2CCSCC2)cc1NC(=O)CN1C(=O)c2ccccc2S1(=O)=O ; Cc1sc(S(=O)(=O)N2CCSCC2)cc1NC(=O)CNC(=O)c1ccccc1 ; Cc1sc(S(=O)(=O)N2CCSCC2)cc1NC(=O)CNC(=O)OCc1ccccc1]
or a pharmaceutically acceptable salt thereof, and combinations thereof.

7. The method of claim 1, wherein SHIP activator compound activates phosphatidylinositol 3,4,5-trisphosphate 5-phosphatase isoform SHIP1 and/or SHIP2.

8. The method of claim 1, wherein SHIP activator compound activates SHIP1.

9. The method of claim 1, wherein treating the subject with the one or more SHIP activator compound is sequential.

10. The method of claim 1, wherein treating the subject with the one or more SHIP activator compound is simultaneous.

11. The method of claim 1, wherein treating with the one or more SHIP activator compound results in reduced inflammation and/or amelioration of symptoms within said subject.

12. The method of claim 1, wherein the one or more SHIP activator compound is selected from the group consisting of: 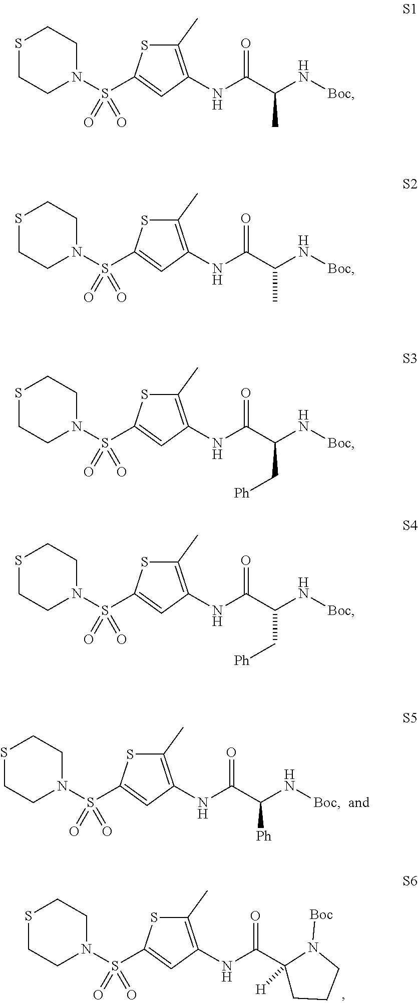 [SMILES: Cc1sc(S(=O)(=O)N2CCSCC2)cc1NC(=O)[C@H](C)NC(=O)OC(C)(C)C ; Cc1sc(S(=O)(=O)N2CCSCC2)cc1NC(=O)[C@@H](C)NC(=O)OC(C)(C)C ; Cc1sc(S(=O)(=O)N2CCSCC2)cc1NC(=O)[C@H](Cc1ccccc1)NC(=O)OC(C)(C)C ; Cc1sc(S(=O)(=O)N2CCSCC2)cc1NC(=O)[C@@H](Cc1ccccc1)NC(=O)OC(C)(C)C ; Cc1sc(S(=O)(=O)N2CCSCC2)cc1NC(=O)[C@@H](NC(=O)OC(C)(C)C)c1ccccc1 ; Cc1sc(S(=O)(=O)N2CCSCC2)cc1NC(=O)[C@@H]1CCCN1C(=O)OC(C)(C)C]
or a pharmaceutically acceptable salt thereof, and combinations thereof.

13. The method of claim 1, where the one or more SHIP activator compound has activity as a SHIP1 agonist.

14. The method of claim 13, wherein the SHIP1 agonist activity is measured by the malachite green phosphatase assay.

15. The method of claim 1, wherein the one or more SHIP activator compound has anti-inflammatory activity as measured by an assay utilizing treatment of cells with lipopolysaccharides (LPS) to induce IL-6 production that is quantified with an enzyme-linked immunosorbent assay (ELISA).

16. A method of agonizing SHIP1 comprising administering 2-(4-Fluorobenzenesulfonamido)-N-[2-methyl-5-(thiomorpholine-4-sulfonyl)thiophen-3-yl]acetamide, or an analog thereof, to an animal in need thereof.

17. A pharmaceutical composition comprising one or more SHIP activators selected from the group consisting of: 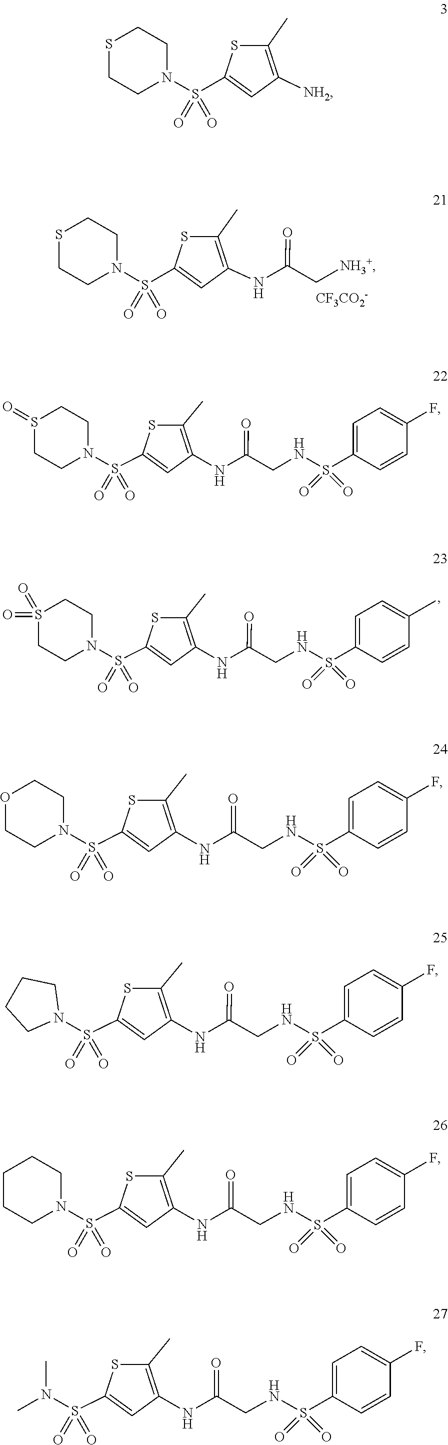 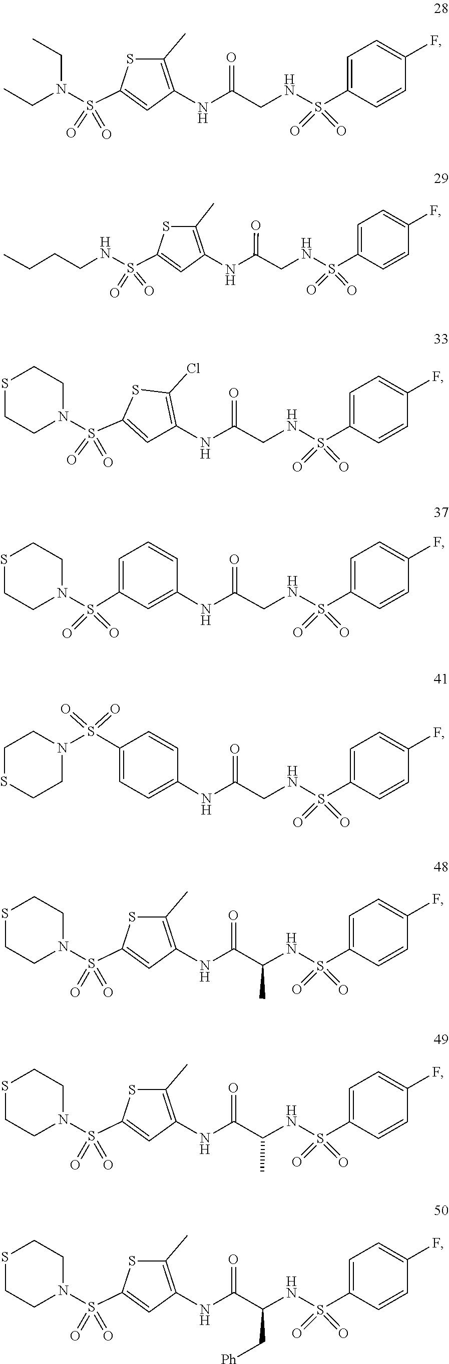 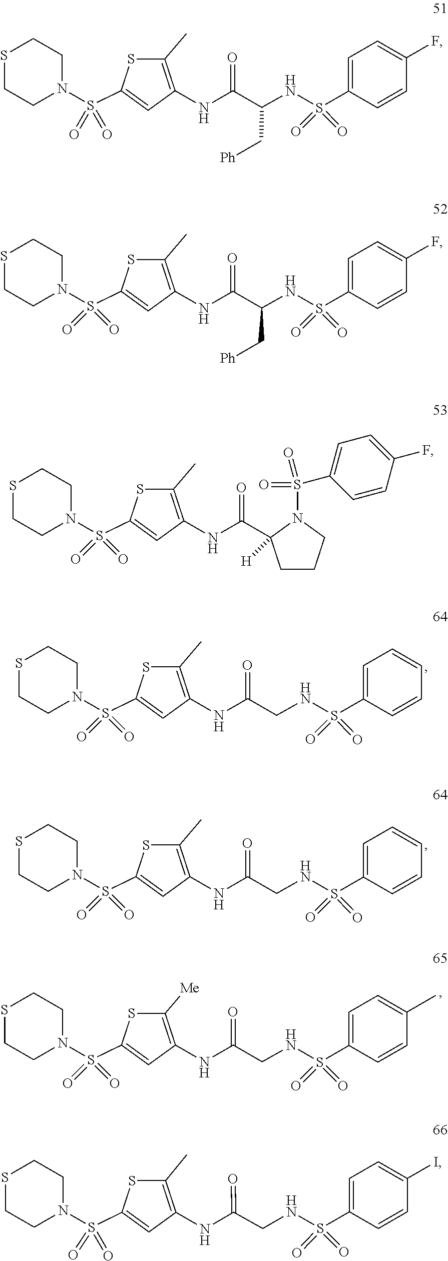 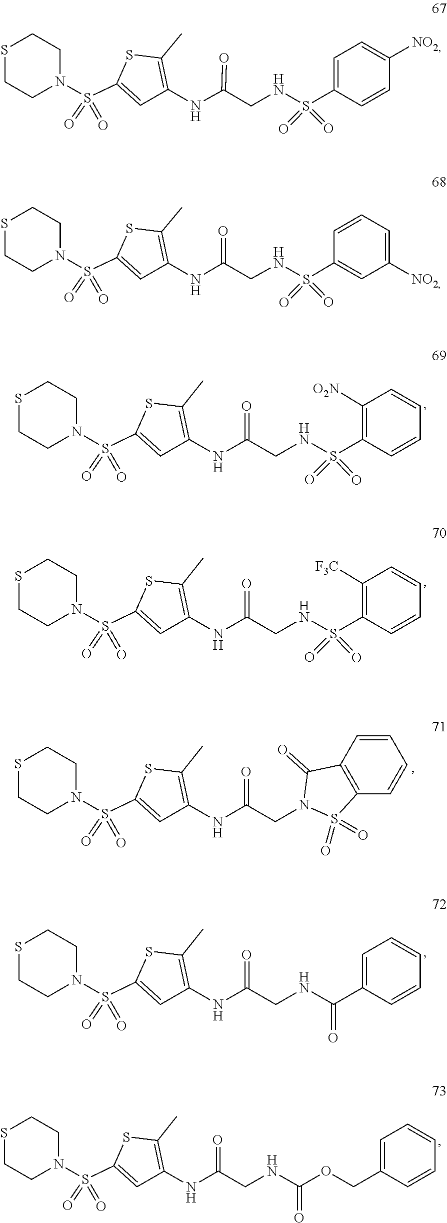 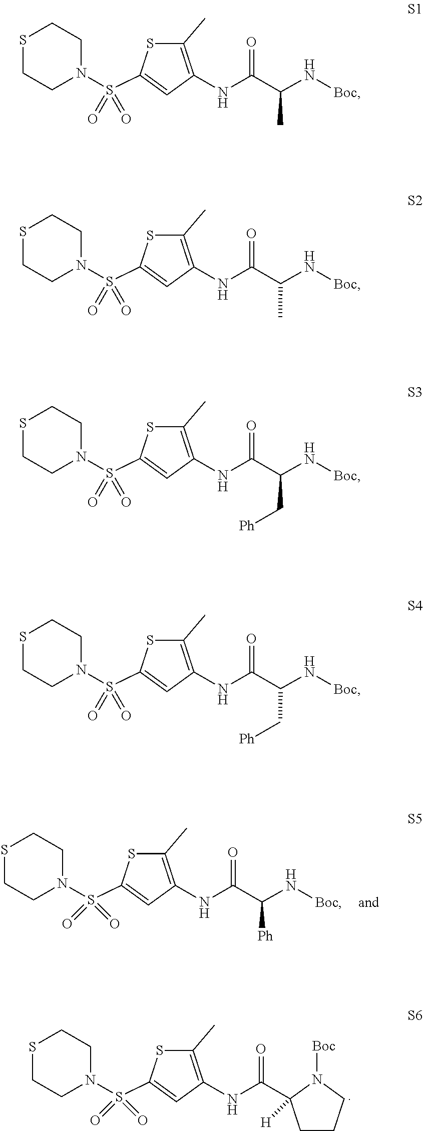
and combinations thereof.

18. The method of claim 1, wherein the one or more SHIP activator compounds are selected from the group consisting of: 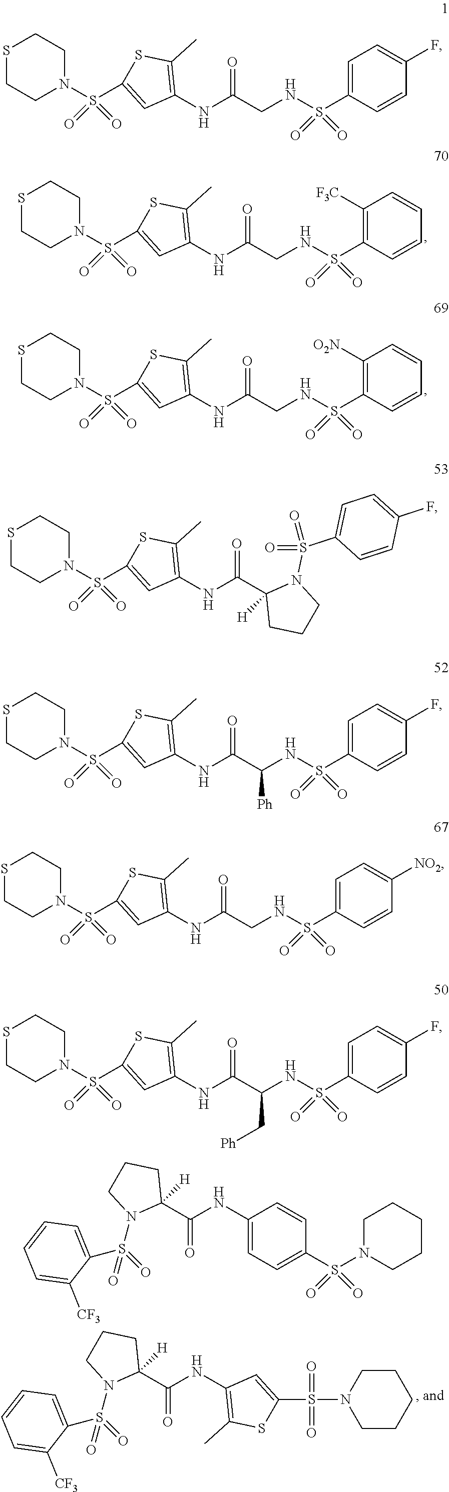 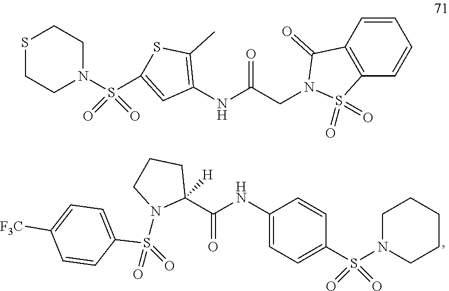
or a pharmaceutically acceptable salt thereof, and combinations thereof.

19. The methods of claim 16, wherein the one or more analog compounds are selected from the group consisting of: 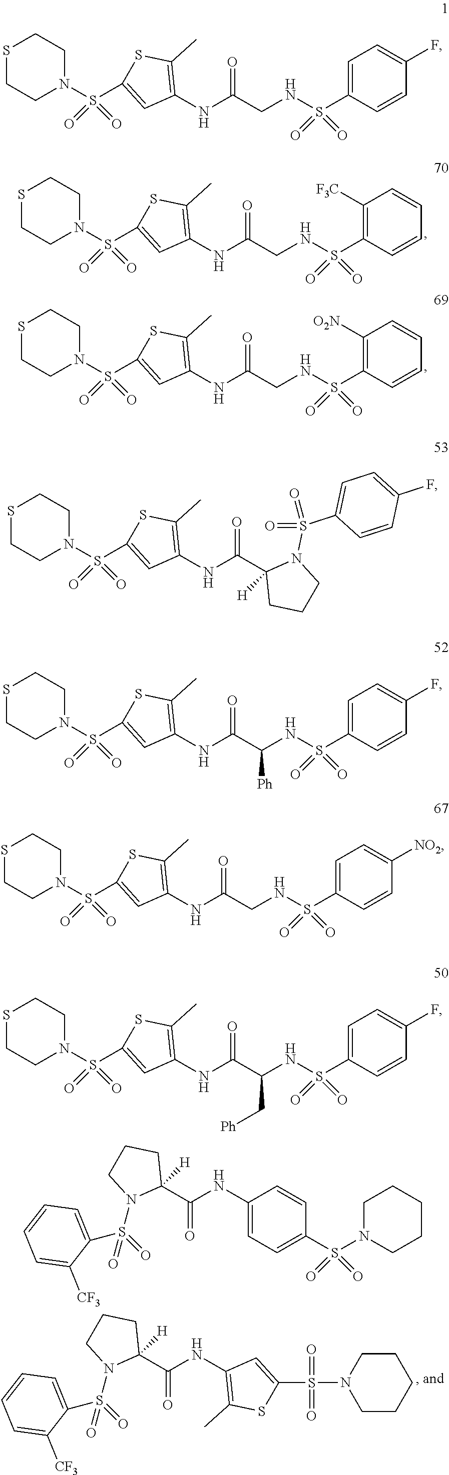 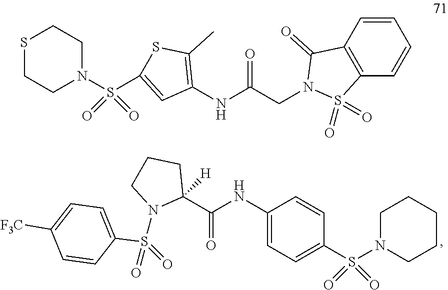
or a pharmaceutically acceptable salt thereof, and combinations thereof.

20. The pharmaceutical composition of claim 17, one or more SHIP activators selected from the group consisting of: 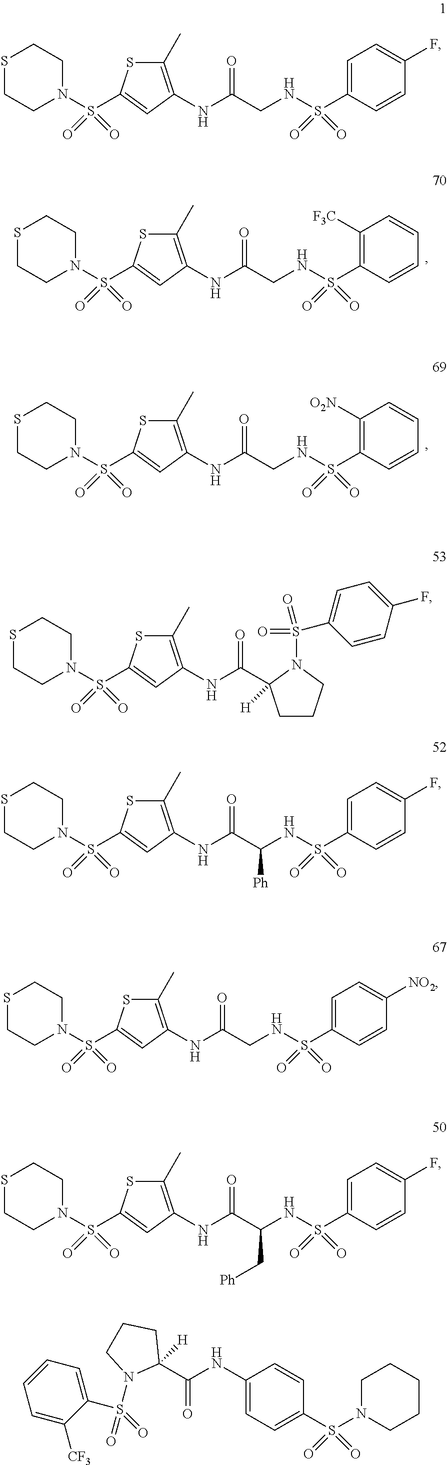 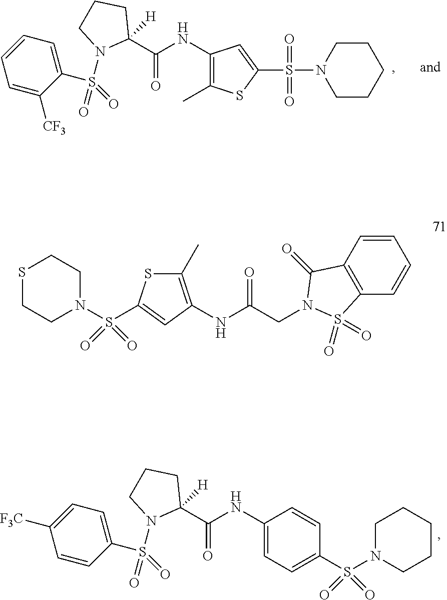 [SMILES: Cc1sc(S(=O)(=O)N2CCCCC2)cc1NC(=O)[C@@H]1CCCN1S(=O)(=O)c1ccccc1C(F)(F)F ; Cc1sc(S(=O)(=O)N2CCSCC2)cc1NC(=O)CN1C(=O)c2ccccc2S1(=O)=O ; O=C(Nc1ccc(S(=O)(=O)N2CCCCC2)cc1)[C@@H]1CCCN1S(=O)(=O)c1ccc(C(F)(F)F)cc1]
or a pharmaceutically acceptable salt thereof, and combinations thereof.
